SEQUENCE LISTING (33 sequences)

SEQ ID NO: 1 - protein, 16 aa, Artificial Sequence
  Synthetic Construct
GSGGGSGGGGSGGGGS

SEQ ID NO: 2 - protein, 6 aa, Artificial Sequence
  Synthetic Construct
GGSGGS

SEQ ID NO: 3 - protein, 6 aa, Artificial Sequence
  Synthetic Construct
SGGGGS

SEQ ID NO: 4 - protein, 12 aa, Artificial Sequence
  Synthetic Construct
GRAGGGGAGGGG

SEQ ID NO: 5 - protein, 6 aa, Artificial Sequence
  Synthetic Construct
GRAGGG

SEQ ID NO: 6 - protein, 1 aa, Artificial Sequence
  Synthetic Construct
  VARIANT 1: Can be present in repeats of any integer
G

SEQ ID NO: 7 - protein, 2 aa, Artificial Sequence
  Synthetic Construct
  VARIANT (1)..(2): Can be present in repeats of any integer
GS

SEQ ID NO: 8 - protein, 5 aa, Artificial Sequence
  Synthetic Construct
  VARIANT (1)..(4): Can be present in repeats of any integer
GSGGS

SEQ ID NO: 9 - protein, 4 aa, Artificial Sequence
  Synthetic Construct
  VARIANT (1)..(4): Can be present in repeats of any integer
GGGS

SEQ ID NO: 10 - protein, 6 aa, Artificial Sequence
  Synthetic Construct
ASTKGP

SEQ ID NO: 11 - protein, 5 aa, Artificial Sequence
  Synthetic Construct
  VARIANT (1)..(5): Can be present in repeats of any integer
GGGGS

SEQ ID NO: 12 - protein, 2 aa, Artificial Sequence
  Synthetic Construct
GG

SEQ ID NO: 13 - protein, 4 aa, Artificial Sequence
  Synthetic Construct
GGSG

SEQ ID NO: 14 - protein, 5 aa, Artificial Sequence
  Synthetic Construct
GGSGG

SEQ ID NO: 15 - protein, 5 aa, Artificial Sequence
  Synthetic Construct
GSGSG

SEQ ID NO: 16 - protein, 5 aa, Artificial Sequence
  Synthetic Construct
GSGGG

SEQ ID NO: 17 - protein, 5 aa, Artificial Sequence
  Synthetic Construct
GGGSG

SEQ ID NO: 18 - protein, 5 aa, Artificial Sequence
  Synthetic Construct
GSSSG

SEQ ID NO: 19 - protein, 15 aa, Artificial Sequence
  Synthetic Construct
GGGGSGGGGSGGGGS

SEQ ID NO: 20 - protein, 5 aa, Artificial Sequence
  Synthetic Construct
GGGGS

SEQ ID NO: 21 - protein, 146 aa, Homo sapiens
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 22 - protein, 223 aa, Artificial Sequence
  Synthetic Construct
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 23 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 24 - protein, 385 aa, Artificial Sequence
  Synthetic Construct
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIGSGGGSGGGGSGGGGSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 25 - protein, 385 aa, Artificial Sequence [sequence omitted]

SEQ ID NO: 26 - protein, 375 aa, Artificial Sequence
  Synthetic Construct
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIASTKGPVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 27 - protein, 375 aa, Artificial Sequence
  Synthetic Construct
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKASTKGPAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 28 - protein, 308 aa, Artificial Sequence
  Synthetic Construct
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIGSGGGSGGGGSGGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 29 - protein, 5 aa, Homo sapiens
ERKCC

SEQ ID NO: 30 - protein, 33 aa, Homo sapiens
MAALQKSVSSFLMGTLATSCLLLLALLVQGGAA

SEQ ID NO: 31 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 32 - protein, 6 aa, Artificial Sequence
  Synthetic Construct
GPGPGP

SEQ ID NO: 33 - protein, 3 aa, Artificial Sequence
  Synthetic Construct
EKR

(19) United States
(12) Patent Application Publication
(10) Pub. No.: US 2023/0079150 A1
(43) Pub. Date: Mar. 16, 2023

(54) METHODS FOR PREVENTION OR TREATMENT OF VIRUS-INDUCED ORGAN INJURY OR FAILURE WITH IL-22 DIMER

(71) Applicant: Evive Biotechnology (Shanghai) Ltd, Shanghai (CN)

(72) Inventors: Zheng YANG, Shanghai (CN); Zhihua HUANG, Shanghai (CN)

(21) Appl. No.: 17/799,627

(22) PCT Filed: Feb. 10, 2021
(86) PCT No.: PCT/CN2021/076519

§ 371 Date: Aug. 12, 2022

(30) Foreign Application Priority Data
Feb. 14, 2020 (CN) PCT/CN2020/075408
Oct. 13, 2020 (CN) PCT/CN2020/120662

(51) Int. Cl. A61K 38/20; A61K 31/7064; A61K 31/427; A61K 38/21; A61P 11/00

(52) U.S. Cl. CPC A61K 38/20; A61K 31/7064; A61K 31/427; A61K 38/212; A61P 11/00

(57) ABSTRACT

Provided is use of IL-22 dimer in prevention or treatment of virus-induced organ injury or failure, such as lung injury or failure, sepsis, septic shock, or multiple organ dysfunction syndrome (MODS) associated with virus infection.

### CROSS REFERENCE TO RELATED APPLICATIONS

This application claims priority benefit of International Patent Application No. PCT/CN2020/075408 filed Feb. 14, 2020 and International Patent Application No. PCT/CN2020/120662 filed Oct. 13, 2020, the contents of each of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 720622001842SEQLIST.TXT, date recorded: Feb. 8, 2021, size: 27 KB).

### FIELD OF THE INVENTION

The present invention relates to use of IL-22 dimer in prevention or treatment of virus-induced organ injury or failure, such as lung injury or failure, sepsis, septic shock, or multiple organ dysfunction syndrome (MODS) associated with virus infection.

### BACKGROUND OF THE INVENTION

Interleukin-22 (IL-22), also known as IL-10 related T cell-derived inducible factor (IL-TIF), is a glycoprotein expressed and secreted by several populations of immune cells, such as activated T cells (mainly CD4+ cells, especially CD28 pathway activated Tₕ1 cells, Tₕ17 cells, and Tₕ22 cells, etc.), IL-2/IL-12 stimulated natural killer cells (NK cells; Wolk et al., J. Immunology, 168:5379-5402, 2002), NK-T cells, neutrophils, and macrophages. The expression of IL-22 mRNA was originally identified in IL-9 stimulated T cells and mast cells in murine, as well as Concanavilin A (Con A) stimulated spleen cells (Dumoutier et al., J. Immunology, 164:1814-1819, 2000). Human IL-22 mRNA is mainly expressed in peripheral T cells upon stimulation by anti-CD3 antibodies or Con A. IL-22 binds to a heterodimeric cell surface receptor composed of IL-10R2 and IL-22R1 subunits. IL-22R1 is specific to IL-22 and is expressed mostly on non-hematopoietic cells, such as epithelial and stromal cells of liver, lung, skin, thymus, pancreas, kidney, gastrointestinal tract, synovial tissues, heart, breast, eye, and adipose tissue.

Pathogenic viral infection can lead to inflammatory cytokine response, which is indispensable for immune protection. However, exaggerated anti-viral response can be harmful to the host, leading to infected organ injury or failure, or even death. Acute viral infections can lead to a cytokine storm, which is the excessive systemic expression of multiple inflammatory mediators such as cytokines, oxygen free radicals, and coagulation factors, caused by rapidly proliferating T-cells or NK cells activated by infected macrophages. For example, the rapid viral replication of Severe Acute Respiratory Syndrome Coronavirus (SARS-CoV) and pandemic influenza (e.g., Influenza A virus subtype H1N1 (H1N1), Influenza A virus subtype H5N1 (H5N1)) results in cytolytic destruction of target cells of the respiratory tract, such as alveolar epithelial cells, leading to rapidly progressive respiratory failure causing acute lung injury (ALI) or acute respiratory distress syndrome (ARDS). In some cases, multiple organ failure (MOF) is also a feature, associated with significant elevation of pro-inflammatory cytokines such as TFNα and IFNβ. The ongoing 2019-2021 coronavirus outbreak is caused by 2019 novel coronavirus (2019-nCov) infection that leads to respiratory infection 2019-nCoV acute respiratory disease. The World Health Organization (WHO) has officially named the disease as “Coronavirus disease 2019” (COVID-19), and the virus as “Severe Acute Respiratory Syndrome Coronavirus 2” (SARS-CoV-2). SARS-CoV-2 infection results in damages and/or failure of the respiratory system, and there seems to be a strong correlation of cytokine storm and the severity of illness in patients, resembling the features seen in SARS and Middle East Respiratory Syndrome (MERS) patients. Many patients admitted to the intensive care unit (ICU), particularly those with severe disease, die from organ failure (not just lung, but also heart, kidney, liver etc.) triggered by cytokine storm.

Multiple organ dysfunction syndrome (MODS), also known as multiple organ failure (MOF), total organ failure (TOF), or multisystem organ failure (MSOF), is altered organ function in an acutely ill patient such that homeostasis cannot be maintained without medical intervention. MODS usually results from uncontrolled inflammatory response triggered by infection, injury (accident, surgery), hypoperfusion, and hypermetabolism. The uncontrolled inflammatory response can lead to sepsis or Systemic Inflammatory Response Syndrome (SIRS). SIRS is an inflammatory state affecting the whole body. It is one of several conditions related to systemic inflammation, organ dysfunction, and organ failure. SIRS is a subset of cytokine storm, in which there is abnormal regulation of various cytokines. The cause of SIRS can be infectious or noninfectious. SIRS is closely related to sepsis. When SIRS is due to an infection, it is considered as sepsis. Noninfectious causes of SIRS include trauma, burns, pancreatitis, ischemia, and hemorrhage. Sepsis is a serious medical condition characterized by a whole-body inflammatory state, and can lead to septic shock. Both SIRS and sepsis can progress to severe sepsis, and eventually MODS, or death. The underline mechanism of MODS is not well understood.

At present, there is no agent that can reverse established organ failure. Therapy is therefore limited to supportive care. Prevention and treatment of organ injury or failure, sepsis, septic shock, and MODS are important to emergency medical conditions, such as injury caused by traffic accident, burns, heart attacks, and severe infective diseases. The development of an effective drug is in urgent need.

The disclosures of all publications, patents, patent applications, and published patent applications referred to herein are incorporated herein by reference in their entirety.

### BRIEF SUMMARY OF THE INVENTION

In one aspect of the present invention, there is provided a method of preventing or treating a virus-induced organ injury or failure in an individual (e.g., human, such as a human of at least about 55 years old), comprising administering to the individual an effective amount of an IL-22 dimer.

In another aspect of the present invention, there is provided a method of protecting an organ (e.g., lung, heart, liver, kidney) from virus-induced organ injury or failure in an individual (e.g., human, such as a human of at least about 55 years old), comprising administering to the individual an effective amount of an IL-22 dimer.

In another aspect of the present invention, there is provided a method of promoting regeneration of injured tissue or organ (e.g., lung, heart, liver, kidney) due to virus infection in an individual (e.g., human, such as a human of at least about 55 years old), comprising administering to the individual an effective amount of an IL-22 dimer.

In another aspect of the present invention, there is provided a method of treating or preventing endothelial dysfunction in an injured tissue or organ (e.g., lung, heart, kidney, liver) due to virus infection in an individual (e.g., human, such as a human of at least about 55 years old), comprising administering to the individual an effective amount of an IL-22 dimer.

In another aspect of the present invention, there is provided a method of reducing inflammation (e.g., cytokine storm, sepsis, SIRS) due to virus infection in an individual (e.g., human, such as a human of at least about 55 years old), comprising administering to the individual an effective amount of an IL-22 dimer.

In some embodiments according to any of the methods described above, the virus-induced organ injury or failure comprises endothelial cell injury, dysfunction, or death. In some embodiments, the injured tissue or organ comprises injured or dysfunctional endothelial cells. In some embodiments, endothelial dysfunction comprises endothelial glycocalyx degradation. In some embodiments, the method comprises preventing and/or reducing endothelial glycocalyx degradation, down-regulating Toll-like Receptor 4 (TLR4) signaling, and/or regenerating endothelial glycocalyx. In some embodiments, the endothelial cell is a pulmonary endothelial cell.

In some embodiments according to any of the methods described above, the virus-induced organ injury or failure is virus-induced lung injury or failure, such as pulmonary fibrosis, pneumonia, acute lung injury (ALI), SARS, MERS, COVID-19, H1N1 swine flu, H5N1 bird flu, or acute respiratory distress syndrome (ARDS). In some embodiments, the virus-induced organ injury or failure is virus-induced sepsis, septic shock, or multiple organ dysfunction syndrome (MODS).

In some embodiments according to any of the methods described above, the virus-induced organ injury or failure is caused by a virus of any one of the Orthomyxoviridae, Filoviridae, Flaviviridae, Coronaviridae, and Poxviridae families. In some embodiments, the virus is an Orthomyxoviridae virus selected from the group consisting of Influenza A virus, Influenza B virus, Influenza C virus, and any subtype or reassortant thereof. In some embodiments, the virus is an Influenza A virus or any subtype or reassortant thereof, such as Influenza A virus subtype H1N1 (H1N1) or Influenza A virus subtype H5N1 (H5N1). In some embodiments, the virus is a Coronaviridae virus selected from the group consisting of alpha coronaviruses 229E (HCoV-229E), New Haven coronavirus NL63 (HCoV-NL63), beta coronaviruses OC43 (HCoV-OC43), coronavirus HKU1 (HCoV-HKU1), Severe Acute Respiratory Syndrome coronavirus (SARS-CoV), Middle East Respiratory Syndrome coronavirus (MERS-CoV), and Severe Acute Respiratory Syndrome Coronavirus 2 (SARS-CoV-2). In some embodiments, the virus is SARS-CoV, MERS-CoV, or SARS-CoV-2. In some embodiments, the virus is a Filoviridae virus selected from Ebola virus (EBOV) and Marburg virus (MARV). In some embodiments, the virus is a Flaviviridae virus selected from the group consisting of Zika virus (ZIKV), West Nile virus (WNV), Dengue virus (DENV), and Yellow Fever virus (YFV).

In some embodiments according to any of the methods described above, comprising administering to the individual an effective amount of another therapeutic agent. In some embodiments, the other therapeutic agent is selected from the group consisting of a corticosteroid, an anti-inflammatory signal transduction modulator, a β2-adrenoreceptor agonist bronchodilator, an anticholinergic, a mucolytic agent, an antiviral agent, an anti-fibrotic agent, hypertonic saline, an antibody, a vaccine, or mixtures thereof. In some embodiments, the antiviral agent is selected from the group consisting of remdesivir, lopinavir/ritonavir (Kaletra®), IFN-α (e.g., IFN-α2a or IFN-α2b), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir, zanamivir, peramivir, amantadine, rimantadine, favipiravir, laninamivir, ribavirin, umifenovir, and any combinations thereof. In some embodiments, the other therapeutic agent is selected from the group consisting of remdesivir, lopinavir/ritonavir (Kaletra®, e.g., tablet), IFN-α (e.g., IFN-α2a or IFN-α2b, via inhalation), favipiravir, lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, and any combinations thereof, and the virus-induced organ injury or failure is induced by SARS-CoV-2. In some embodiments, the other therapeutic agent is remdesivir and the virus-induced organ injury or failure is induced by SARS-CoV-2. In some embodiments, the other therapeutic agent is lopinavir/ritonavir (Kaletra®, e.g., tablet) and IFN-α (e.g., via inhalation), and the virus-induced organ injury or failure is induced by SARS-CoV-2. In some embodiments, the other therapeutic agent is selected from the group consisting of oseltamivir, zanamivir, peramivir, favipiravir, umifenovir (Arbidol®), teicoplanin derivatives, benzo-heterocyclic amine derivative, pyrimidine, baloxavir marboxil, lopinavir/ritonavir (Kaletra®, e.g., tablet), IFN-α (e.g., e.g., IFN-α2a, IFN-α2b, via inhalation), and any combinations thereof, and the virus-induced organ injury or failure is induced by H1N1 or H5N1. In some embodiments, the other therapeutic agent is lopinavir/ritonavir (Kaletra®, e.g., tablet) and IFN-α (e.g., IFN-α2a, IFN-α2b, via inhalation), and the virus-induced organ injury or failure is induced by H1N1 or H5N1. In some embodiments, the anti-fibrotic agent is selected from the group consisting of nintedanib, pirfenidone, and N-Acetylcysteine (NAC). In some embodiments, the IL-22 dimer is administered simultaneously or sequentially with the other therapeutic agent.

In some embodiments according to any of the methods described above, the IL-22 dimer comprises two monomeric subunits, and wherein each monomeric subunit comprises an IL-22 monomer and a dimerization domain. In some embodiments, the IL-22 monomer is connected to the dimerization domain via an optional linker. In some embodiments, the linker comprises the sequence of any one of SEQ ID NOs: 1-20 and 32, such as SEQ ID NO: 1 or 10. In some embodiments, the linker is about 6 to about 30 (e.g., about 6 to about 15) amino acids in length. In some embodiments, the dimerization domain comprises at least two (e.g., 2, 3, 4) cysteines capable of forming intermolecular disulfide bonds. In some embodiments, the dimerization domain comprises at least a portion of an Fc fragment. In some embodiments, the Fc fragment comprises CH2 and CH3 domains. In some embodiments, the Fc fragment comprises the sequence of SEQ ID NO: 22 or 23. In some embodiments, the IL-22 monomer comprises the sequence of SEQ ID NO: 21. In some embodiments, the IL-22 monomer is N-terminal to the dimerization domain. In some embodiments, the IL-22 monomer is C-terminal to the dimerization domain. In some embodiments, each monomeric subunit comprises the sequence of any of SEQ ID NOs: 24-27, such as SEQ ID NO: 24.

In some embodiments according to any of the methods described above, the effective amount of the IL-22 dimer is about 2 μg/kg to about 200 μg/kg, such as about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg.

In some embodiments according to any of the methods described above, the IL-22 dimer is administered intravenously, intrapulmonarily, or via inhalation (e.g., through mouth or nose) or insufflation. In some embodiments, the IL-22 dimer is administered intravenously.

In some embodiments according to any of the methods described above, the IL-22 dimer is administered at least once a week. In some embodiments, the IL-22 dimer is administered on day 1 and day 6 of a 10-day treatment cycle. In some embodiments, the IL-22 dimer is administered on day 1 and day 8 of a 14-day treatment cycle.

In some embodiments according to any of the methods described above, the individual (e.g., human) is at least about 55 years old (e.g., at least about any of 60, 65, 70, 75, 80, 85, 90 years old, or older).

In some embodiments according to any of the methods described above, the method further comprises selecting the individual based on that the individual is at least about 55 years old (e.g., at least about any of 60, 65, 70, 75, 80, 85, 90 years old, or older).

Also provided are compositions, kits, and articles of manufactures comprising any of the IL-22 dimers described herein for use in any methods described herein.

These and other aspects and advantages of the present invention will become apparent from the subsequent detailed description and the appended claims. It is to be understood that one, some, or all of the properties of the various embodiments described herein may be combined to form other embodiments of the present invention.

### BRIEF DESCRIPTION OF THE DRAWINGS

FIG. 1 depicts an exemplary IL-22 dimer according to the present invention. In the figure, “-” represents a linker, and the oval-shaped object labeled with “IL-22” represents an IL-22 monomer.

FIGS. 2A-2B depict exemplary IL-22 dimers according to the present invention. In the figures, “-” represents an amino acid linker and the oval-shaped object labeled with “IL-22” represents an IL-22 monomer. As illustrated in FIG. 2A, the oval-shaped object labeled with “C” represents a carrier protein wherein the IL-22 is disposed at the N-terminal of the carrier protein. As illustrated in FIG. 2B, the half oval-shaped object labeled with “Fc” represents an Fc fragment as a dimerization domain, showing a dimer is formed by the coupling of two Fc fragments via disulfide bond(s).

FIGS. 3A-3B depict exemplary IL-22 dimers according to the present invention. In the figures, “-” represents an amino acid linker, the oval-shaped object labeled with “IL-22” represents an IL-22 monomer. As illustrated in FIG. 3A, the oval-shaped object labeled with “C” represents a carrier protein wherein the IL-22 is disposed at the C-terminal of the carrier protein. As illustrated in FIG. 3B, the half oval-shaped object labeled with “Fc” represents an Fc fragment as a dimerization domain, showing a dimer is formed by the coupling of two Fc fragments via disulfide bond(s).

FIG. 4 depicts survival rates of mice model of H1N1 infection in treatment and control groups over time.

FIGS. 5A-5C depict H&E staining of lung tissues from Model control group (FIG. 5A), Oseltamivir treatment group (FIG. 5B), and (F-652+oseltamivir) treatment group (FIG. 5C) on Day 5 post-H1N1 infection, under 100× magnification.

FIGS. 6A-6C depict H&E staining of lung tissues from Model control group (FIG. 6A), Oseltamivir treatment group (FIG. 6B), and (F-652+oseltamivir) treatment group (FIG. 6C) on Day 14 post-H1N1 infection, under 100× magnification.

FIG. 7A depicts a comparison of glycocalyx staining intensity in control HUVECs, LPS exposed, LPS and F-652 exposed, and F-652 only exposed. Representative images of all 4 groups are shown. FIG. 7B depicts a comparison of IL-22Ra1 relative expression in all 4 groups of HUVECs.

FIG. 8A depicts a comparison of phosphorylated STAT3:total STAT3 ratio in control HUVECS and F-652 treated HUVECS (left), and an SDS-Polyacrylamide gel electrophoresis western blot quantifying phosphorylated STAT3 and total STAT3 (right). FIG. 8B shows relative expression of matrix metalloproteinase-1 (MMP-1), MMP-2, MMP-9, and MMP-14 mRNA levels in control, LPS exposed, LPS and F-652 exposed, and F-652 only exposed HUVECs.

FIG. 9 shows relative expression of TIMP-1, TIMP-2, Exostosin-1, and Exostosin-2 mRNA levels in control, LPS exposed, LPS and F-652 exposed, and F-652 only exposed HUVECs.

FIG. 10 shows relative expression of TLR4, MYD88, TIRAP, and IRAK4 mRNA levels in control, LPS exposed, LPS and F-652 exposed, and F-652 only exposed HUVECs.

FIG. 11 shows relative expression of TRAM, TRAF6, IRAK1, and TRIF mRNA levels in control, LPS exposed, LPS and F-652 exposed, and F-652 only exposed HUVECs.

FIG. 12 shows that mice with low-dose LPS injury have decreased cellular influx of neutrophils and macrophages into the lungs when treated with F-652 as shown in BAL cell counts. There was no difference seen in total cell counts and lymphocyte counts.

FIG. 13 shows that mice with high-dose LPS injury have decreased cellular influx into the lungs when treated with F-652 as shown in BAL cell counts. F-652 treated mice have decreased total cell counts, neutrophil counts, lymphocyte counts, and macrophage counts.

FIG. 14 shows that mice with high-dose LPS injury have decreased inflammation in the lungs when treated with F-652 as shown in BAL inflammatory mediators. F-652 treated mice have decreased Interleukin-6, TNF-alpha, G-CSF, and Interleukin-10.

FIGS. 15A-15C show that mice with high-dose LPS injury have less severe damage to the lungs when treated with F-652 as seen with histopathology scores graded by a blinded reviewer (FIG. 15A). Representative images of lung tissue are shown F-652 treated (FIG. 15B) and Sham animals (FIG. 15C).

FIG. 16 shows that F-652 treated mice have improved preservation of the endothelial glycocalyx in alveolar capillaries as compared to sham animals. Endothelial glycocalyx staining intensity was increased in the alveolar capillaries in F-652 treated mice after low-dose LPS injury. Endothelial glycocalyx staining intensity was not different for F-652 treated mice in high-dose LPS injury.

FIG. 17 shows that treatment with F-652 (human IL-22-Fc) results in increased endogenous mouse IL-22. Exogenous human IL-22 was detected in the BAL of treated mice, demonstrating that exogenous F-652 is reaching the lung. Endogenous mouse F-652 was not increased in F-652 treated after high-dose LPS injury.

FIG. 18A shows viral copies in SARS-CoV-2 infected primary human bronchial epithelial (HBE) cells as reflected by subgenomic-N (sgm-N) RNA copies, either pre-treated with F-652 or post-treated with F-652. HBE cells not infected by SARS-CoV-2, or SARS-CoV-2 infected HBE cells without treatment seaved as controls. Both pre-treatment and post-treatment with F-652 showed significantly lower copies of sgm-N RNA copies compared to no F-652 treatment group (p<0.05, ANOVA, Tukey's multiple comparisons test). FIG. 18B shows % of RNA-seq reads that map to SARS-CoV-2 open reading frame (ORF) in different groups of SARS-CoV-2 infected HBE cells.

FIG. 19A shows average body weight post H1N1 infection in young and aged mice, compared to Day 0 body weight. FIG. 19B shows survival rate of young and aged mice post H1N1 infection. “****” indicates statistical significance.

FIGS. 20A and 20C show average body weight post H1N1 infection in young (FIG. 20A) and aged (FIG. 20C) mice, compared to Day 0 body weight, either treated with PBS control or F-652. FIGS. 20B and 20D show survival rate of young (FIG. 20B) and aged (FIG. 20D) mice post H1N1 infection, either treated with PBS control or F-652.

FIG. 21 shows the number of lung infiltrating neutrophils and inflammatory monocytes from lung tissues of young and old H1N1 infected mice treated with PBS or F-652. “***” and “**” indicate statistical significance.

FIG. 22 shows the number of parenchymal (pathogenic) CD8+ T cells in lung tissues of young and old H1N1 infected mice treated with PBS or F-652. Left panels indicate total CD8+ T cell numbers; middle panels indicate CD8+ T cells expressing CD69+; right panels indicate CD8+ T cells expressing CD69+ and CD103+. “***” and “*” indicate statistical significance.

FIG. 23 shows lung histology images (40×resolution) from lungs of aged H1N1-infected mice, stained with hematoxylin and eosin (H&E), Masson's Trichrome, Sirius Red, or Periodic acid-Schiff (PAS).

FIG. 24 shows exemplary experimental set up to study lung functions in mice.

FIG. 25 shows tissue dampening (G) measured by forced oscillation technique (FOT) in young (top panels) and aged (bottom panels) H1N1 infected mice treated (F-652) or not treated (PBS) prior to (“baseline” panels) and following (“full capacity” panels) airway recruitment maneuver. “*” indicates statistical significance.

FIGS. 26A-26B show normalized tissue dampening (capacity G/baseline G reflected as “% ΔG”) to determine % tissue dampening (airway resistance in parenchyma) in young (FIG. 26A) and aged (FIG. 26B) H1N1-infected mice, either treated with F-652 or PBS control. “*” indicates statistical significance.

FIG. 27 shows input impedance (top panels) and reactance (bottom panels) measured with FOT on the flexiVent® prior to (“baseline” panels) and following (“post-airway” panels) airway recruitment maneuver in aged H1N1-infected mice treated (F-652) or not treated (PBS). “*” indicates statistical significance.

FIGS. 28A-28B show input impedance (Re Zrs) measured with FOT on the flexiVent® prior to airway recruitment maneuver in aged (FIG. 28A) and young (FIG. 28B) H1N1-infected mice treated (F-652) or not treated (PBS). “*” indicates statistical significance.

FIGS. 29A-29B show input impedance (Re Zrs) measured with FOT on the flexiVent® following airway recruitment maneuver in aged (FIG. 29A) and young (FIG. 29B) H1N1-infected mice treated (F-652) or not treated (PBS). “*” indicates statistical significance.

FIGS. 30A-30B show input impedance (Re Zrs) normalized at each frequency as reflected by % (capacity Re Zrs/baseline Re Zrs) for aged (FIG. 30A) and young (FIG. 30B) H1N1-infected mice treated (F-652) or not treated (PBS). “*” indicates statistical significance.

FIGS. 31A-31B show input impedance (Re Zrs; FIG. 31A) and normalized input impedance (% Re Zrs) at each frequency (FIG. 31B) measured with FOT on the flexiVent® in aged H1N1-infected mice treated (F-652) or not treated (PBS), reflecting increasing of airway diameter. “*” indicates statistical significance.

FIGS. 32A-32C show static compliance (Cst) determined in aged mice treated with F-652 or PBS control from pressure-volume (PV) loop maneuvers during tidal breathing (FIG. 32A), post-airway recruitment (FIG. 32B), and normalized to each other (FIG. 32C). “*” indicates statistical significance.

FIGS. 33A-33B show hydroxyproline content from right lung lobes in young (FIG. 33A) and aged (FIG. 33B) mice, either not infected by H1N1 (“naïve”), treated with PBS control, or treated with F-652. “*” indicates statistical significance.

### DETAILED DESCRIPTION OF THE INVENTION

The present invention provides methods of preventing or treating a virus-induced organ injury or failure (e.g., necrosis, lung injury or failure such as pulmonary fibrosis, pneumonia, ALI, SARS, MERS, COVID-19, H1N1 swine flu, H5N1 bird flu, or ARDS, sepsis, septic shock, MODS, death) in an individual (e.g., human, such as a human of at least about 55 years old), comprising administering to the individual an effective amount (e.g., about 2 μg/kg to about 200 μg/kg) of an IL-22 dimer. In some embodiments, the present disclosure provides a method for preventing worsening of, arresting and/or ameliorating at least one symptom of a viral infection in an individual in need thereof (e.g., endothelial dysfunction, endothelial glycocalyx (EGX) degradation, cytokine storm, MODS), preventing damage to said individual or an organ or tissue of said individual, or promoting injured tissue/organ regeneration (e.g., regenerating endothelial cells and/or EGX), emanating from or associated with said viral infection, and preventing death, comprising administering to the individual an effective amount of an IL-22 dimer. In some embodiments, the IL-22 dimer comprises two monomeric subunits, wherein each monomeric subunit comprises an IL-22 monomer and a dimerization domain. In some embodiments, each monomeric subunit comprises the sequence of any of SEQ ID NOs: 24-27 (such as SEQ ID NO: 24). In some embodiments, the IL-22 dimer is administered intravenously, intrapulmonarily, or via inhalation or insufflation. In some embodiments, the methods described herein are particularly effective in preventing or treating a virus-induced organ (e.g., lung) injury or failure in an aged individual (e.g., a human of at least about 55 years old) compared to a young individual (e.g., less than about 20 years old).

The ongoing COVID-19 causes damages and/or failure of the respiratory system, and there seems to be a strong correlation of cytokine storm and the severity of illness in patients, resembling the features seen in SARS and MERS patients. Many patients admitted to the ICU, particularly those with severe disease, die from organ failure (not just lung, but also heart, kidney, liver etc.) triggered by cytokine storm. Besides, older individuals have significantly worse outcomes. Emerging evidence has suggested that COVID-19 survivors exhibit persistent impairment of lung function due to the development of lung fibrosis (Y H. Xu et al. J Infect. 2020 April; 80(4):394-400; S. Zhou et al. AJR Am J Roentgenol. 2020 June; 214(6):1287-1294; M. Hosseiny et al. AJR Am J Roentgenol. 2020 May; 214(5):1078-1082). SARS-CoV-2 binds to angiotensin-converting enzyme 2 (ACE2), which is abundantly present in human epithelia of the lung and vascular endothelial cells. Endothelial glycocalyx (EGX) covers the luminal surface of endothelial cells and regulates endothelial permeability. Disruption of the EGX is observed early in critically ill COVID-19 patients. Endothelial cell dysfunction and EGX damage have been implicated as a major player in COVID-19 (K Stahl et al. Am J Respir Crit Care Med. 2020 October; 202(8):1178-1181; M. Ackermann et al. N Engl J Med. 2020 July; 383(2):120-128; M. Yamaoka-Tojo. Biomed J. 2020 October; 43(5): 399-413; A. Huertas et al. Eur Respir J. 2020 July; 56(1): 2001634; J. N. Conde et al. mBio. 2020 December; 11(6):e03185-20).

IL-22 has demonstrated some therapeutic effects in treating metabolic disease, fatty liver, hepatitis (e.g., viral hepatitis, alcoholic hepatitis), MODS, neurological disorder, pancreatitis, graft versus host disease (GvHD), necrotizing enterocolitis (NEC), and inflammatory bowel disease (IBD). See, e.g., WO2017181143, U.S. Pat. No. 8,956,605, U.S. Ser. No. 10/543,169, U.S. Pat. Nos. 8,945,528, 9,629,898, 7,696,158, 7,718,604, 7,666,402, 9,352,024, U.S. Ser. No. 10/786,551, US20160271221, US20160287670, and ClinicalTrials.gov Identifier: NCT02655510, the contents of which are incorporated herein by reference in their entirety. IL-22 has also demonstrated some therapeutic effects or potential effects in treating pulmonary diseases. See, e.g., J. M. Felton et al. Thorax 2018; 73:1081-1084; M. Pichavant et al. EBioMedicine 2 (2015) 1686-1696; P. Fang et al. Plos One (2014). 9(9): e107454; A. Broquet et al. Scientific Reports. (2017)7: 11010; S. Das et al. iScience (2020) 23:101256; S. Ivanov et al. Journal of Virology (2013) 87(12):6911-6924; R. N. Abood et al. Mucosal Immunol. (2019) 12(5):1231-1243; G. Trevejo-Nunez et al. JImmunol. (2016) 197(5):1877-1883; G. Trevejo-Nunez et al. Infection and Immunity (2019) 87(11):e00550-19; K. D. Hebert et al. Respiratory Research (2019) 20:184; K. D. Hebert et al. Mucosal Immunology (2020) 13:64-74; D. A. Pociask et al. The American Journal of Pathology, 182(4):1286-1296, the contents of which are incorporated herein by reference in their entirety.

IL-22 dimers described herein can be effective in preventing or treating virus-induced organ (e.g., lung) injury or failure (e.g., pulmonary fibrosis), by exhibiting i) antiviral activity (e.g., reducing viral load), ii) anti-inflammatory and tissue-protective role of preventing tissue and/or organ damage from infiltrated inflammatory cells (e.g., cytotoxic T cells (CTLs), monocytes, neutrophils, macrophages, NK cells) attracted by excessive systemic expression of multiple inflammatory mediators, down-regulation of inflammatory mediators (e.g., CCL4), down-regulation of pro-inflammatory pathways such as TLR4 signaling, iii) endothelial-protective role (e.g., preventing or reducing EGX shedding and/or damage; regenerating endothelial cells and/or EGX; preventing or reducing endothelial dysfunction, injury, and/or death; protecting adherens junctions between endothelial cells and/or endothelial cell surface proteins, such as down-regulating extracellular proteinase (e.g., MMPs) expression, up-regulating extracellular matrix protein expression; down-regulating TLR4 signaling; preventing or reducing protein leakage), and iv) reducing or preventing collagen deposition, etc. The IL-22 dimers described herein also have much longer in vivo half-life compared to IL-22 monomers, which can greatly reduce administration frequency and patient cost. Further, the IL-22 dimers described herein can be administered safely with minimal or no adverse event, e.g., via IV administration. Upon an extensive and thorough study, the inventors have surprisingly found that IL-22 dimer has an outstanding effect in the manufacture of a medicament for intravenous administration. It was surprisingly found that an IL-22 dimer, specifically, a dimeric complex of IL-22-Fc monomeric subunits, shows significantly lower toxicity when administered intravenously as compared to subcutaneous administration. Specifically, when a dimeric complex of IL-22-Fc monomeric subunits is administered subcutaneously to an individual at a dosage of about 2 μg/kg, delayed adverse events of the injection site, such as dry skin, erythema and nummular eczema were observed after dosing. On the other hand, the dimeric complex of IL-22-Fc monomeric subunits administered intravenously to an individual demonstrated excellent safety profile. No adverse event of the injection site and skin was observed at doses of about 2 μg/kg or 10 μg/kg. Even at doses as high as about 30 μg/kg to about 45 μg/kg, only limited adverse events such as dry skin, eye pruritus, erythematous rash were observed. The administration of IL-22 dimer also did not lead to an increased serum level of inflammatory cytokines in human.

### I. Definitions

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Current Protocols in Molecular Biology or Current Protocols in Immunology, John Wiley & Sons, New York, N.Y. (2009); Ausubel et al., Short Protocols in Molecular Biology, 3rd ed., John Wiley & Sons, 1995; Sambrook and Russell, Molecular Cloning: A Laboratory Manual (3rd Edition, 2001); Maniatis et al., Molecular Cloning: A Laboratory Manual (1982); DNA Cloning: A Practical Approach, vol. I&II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with organ injury or failure (e.g., pulmonary fibrosis, pneumonia, ALI, ARDS, SARS, MERS, COVID-19, H1N1 swine flu, H5N1 bird flu, sepsis, septic shock, MODS) are mitigated or eliminated, including, but are not limited to, reducing the proliferation of (or destroying) infectious virus, decreasing symptoms resulting from the disease (e.g., respiratory failure, lung fibrosis, cytokine storm, endothelial dysfunction or death, EGX degradation), increasing the quality of life of those suffering from the disease, decreasing the dose of other medications required to treat the disease, and/or prolonging survival of individuals.

As used herein, an “effective amount” refers to an amount of an agent or drug effective to treat a disease or disorder in a subject. In the case of virus-induced organ injury or failure, the effective amount of the agent may inhibit (i.e., reduce to some extent and preferably abolish) virus activity; control and/or attenuate and/or inhibit inflammation or a cytokine storm induced by said viral pathogen; prevent worsening, arrest and/or ameliorate at least one symptom of said viral infection or damage to said subject or an organ or tissue of said subject, emanating from or associated with said viral infection; control, reduce, and/or inhibit cell necrosis in infected and/or non-infected tissue and/or organ; and/or control, ameliorate, and/or prevent the infiltration of inflammatory cells (e.g., NK cells, cytotoxic T cells, neutrophils, monocytes, macrophages) in infected or non-infected tissues and/or organs. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, an “individual” or a “subject” refers to any organism, such as a mammal, including, but not limited to, human, bovine, horse, feline, canine, rodent, or primate. In some embodiments, the individual is a human.

The term “antibody” is used in the broadest sense and specifically covers monoclonal antibodies (including full-length monoclonal antibodies), multispecific antibodies (e.g., bispecific antibodies), and antibody fragments so long as they exhibit the desired biological activity or function. As used herein, the terms “immunoglobulin” (Ig) and “antibody” are used interchangeably.

The term “constant domain” refers to the portion of an immunoglobulin molecule having a more conserved amino acid sequence relative to the other portion of the immunoglobulin, the variable domain, which contains the antigen-binding site. The constant domain contains the C_{H}1, C_{H}2 and C_{H}3 domains (collectively, CH) of the heavy chain and the CHL (or CL) domain of the light chain.

The term IgG “isotype” or “subclass” as used herein is meant any of the subclasses of immunoglobulins defined by the chemical and antigenic characteristics of their constant regions. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000).

“Covalent bond” as used herein refers to a stable bond between two atoms sharing one or more electrons. Examples of covalent bonds include, but are not limited to, peptide bonds and disulfide bonds. As used herein, “peptide bond” refers to a covalent bond formed between a carboxyl group of an amino acid and an amine group of an adjacent amino acid. A “disulfide bond” as used herein refers to a covalent bond formed between two sulfur atoms, such as a combination of two Fc fragments by one or more disulfide bonds. One or more disulfide bonds may be formed between the two fragments by linking the thiol groups in the two fragments. In some embodiments, one or more disulfide bonds can be formed between one or more cysteines of two Fc fragments. Disulfide bonds can be formed by oxidation of two thiol groups. In some embodiments, the covalent linkage is directly linked by a covalent bond. In some embodiments, the covalent linkage is directly linked by a peptide bond or a disulfide bond.

As use herein, the term “binds”, “specifically binds to” or is “specific for” refers to measurable and reproducible interactions such as binding between a target and a receptor, which is determinative of the presence of the target in the presence of a heterogeneous population of molecules including biological molecules. For example, a ligand (e.g., IL-22) that binds to or specifically binds to a receptor (e.g., IL-22R) is a ligand that binds this receptor with greater affinity, avidity, more readily, and/or with greater duration than it binds to other receptors. In one embodiment, the extent of binding of a ligand to an unrelated receptor is less than about 10% of the binding of the ligand to the receptor as measured, e.g., by a radioimmunoassay (RIA). In some embodiments, a ligand that specifically binds to a receptor has a dissociation constant (K_{d}) of <1 μM, <100 nM, <10 nM, <1 nM, or <0.1 nM. In some embodiments, a ligand specifically binds to a binding domain of a receptor conserved among the protein from different species. In another embodiment, specific binding can include, but does not require exclusive binding.

As used herein, “Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table A. Amino acid substitutions may be introduced into an antibody of interest and the products screened for a desired activity, e.g., retained/improved target binding, decreased immunogenicity, or improved ADCC or CDC.

TABLE A
Amino acid substitutions

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |
| Leu (L) | Norleucine; Ile; Val; Met; Ala; Phe |

Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class.

As used herein, the “C terminus” of a polypeptide refers to the last amino acid residue of the polypeptide which donates its amine group to form a peptide bond with the carboxyl group of its adjacent amino acid residue. “N terminus” of a polypeptide as used herein refers to the first amino acid of the polypeptide which donates its carboxyl group to form a peptide bond with the amine group of its adjacent amino acid residue.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “cell” includes the primary subject cell and its progeny.

The term “cytokine storm,” also known as a “cytokine cascade” or “hypercytokinemia,” is a potentially fatal immune reaction typically consisting of a positive feedback loop between cytokines and immune cells, with highly elevated levels of various cytokines (e.g. INF-γ, IL-10, IL-6, CCL2, etc.).

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat disease of type X means the method is used to treat disease of types other than X.

The term “about X-Y” used herein has the same meaning as “about X to about Y.”

As used herein and in the appended claims, the singular forms “a,” “or,” and “the” include plural referents unless the context clearly dictates otherwise.

### II. Methods of Preventing or Treating a Virus-Induced Organ Injury or Failure with IL-22 Dimer

The present invention provides methods of preventing or treating a virus-induced organ injury or failure (e.g., necrosis, lung injury or failure such as pulmonary fibrosis, pneumonia, ALI, SARS, MERS, COVID-19, H1N1 swine flu, H5N1 bird flu, or ARDS, sepsis, septic shock, MODS, death) in an individual (e.g., human, such as a human of at least about 55 years old), comprising administering to the individual an effective amount (e.g., about 2 μg/kg to about 200 μg/kg) of an IL-22 dimer. The present invention also provides methods of protecting an organ from virus-induced organ injury or failure (e.g., necrosis, lung injury or failure such as pulmonary fibrosis, pneumonia, ALI, SARS, MERS, COVID-19, H1N1 swine flu, H5N1 bird flu, or ARDS, sepsis, septic shock, MODS) in an individual (e.g., human, such as a human of at least about 55 years old), comprising administering to the individual an effective amount (e.g., about 2 μg/kg to about 200 μg/kg) of an IL-22 dimer. The present invention also provides methods of reducing inflammation due to virus infection in an individual (e.g., human, such as a human of at least about 55 years old), comprising administering to the individual an effective amount (e.g., about 2 μg/kg to about 200 μg/kg) of an IL-22 dimer. The present invention also provides methods of promoting regeneration of injured tissue or organ (e.g., lung, heart, liver, kidney) due to virus infection (e.g., SARS-CoV, MERS-CoV, SARS-CoV-2) in an individual (e.g., human, such as a human of at least about 55 years old), comprising administering to the individual an effective amount (e.g., about 2 μg/kg to about 200 μg/kg) of an IL-22 dimer. The present invention also provides methods of treating or preventing endothelial dysfunction in an injured tissue or organ (e.g., lung, heart, kidney, liver) due to virus infection (e.g., SARS-CoV, MERS-CoV, SARS-CoV-2) in an individual (e.g., human, such as a human of at least about 55 years old), comprising administering to the individual an effective amount (e.g., about 2 μg/kg to about 200 μg/kg) of an IL-22 dimer. In some embodiments, the virus-induced organ injury or failure comprises endothelial cell injury, dysfunction, or death. In some embodiments, the injured tissue or organ comprises injured or dysfunctional endothelial cells. In some embodiments, endothelial dysfunction comprises EGX degradation. In some embodiments, the method comprises preventing and/or reducing EGX degradation, down-regulating TLR4 signaling, and/or regenerating endothelial cells and/or EGX. In some embodiments, the endothelial cell is a pulmonary endothelial cell. In some embodiments, the methods described herein prevent worsening of, arrest and/or ameliorate at least one symptom of a viral infection in an individual in need thereof, prevent damage to said individual or an organ or tissue of said individual, or promote injured tissue/organ regeneration, emanating from or associated with said viral infection, and/or prevent death. In some embodiments, the methods described herein can achieve one or more of the following: (a) reducing the levels of amylase, lipase, triglyceride (TG), aspartate transaminase (AST), and/or alanine transaminase (ALT) in vivo, such as reducing at least about 10% (including for example at least about any of 20%, 30%, 40%, 50%, 60%, 70%, 80%, 90%, or 100%); (b) controlling, ameliorating, and/or preventing tissue and/or organ (e.g., lung, heart, kidney, liver) injury or failure (e.g., pulmonary fibrosis) in vivo, such as induced by virus infection; (c) controlling, reducing, and/or inhibiting cell necrosis in vitro and/or in vivo (such as reducing at least about 10% (including for example at least about any of 20%, 30%, 40%, 50%, 60%, 70%, 80%, 90%, or 100%) cell necrosis), such as necrosis in infected and/or non-infected tissue and/or organ (e.g., lung, heart, kidney, liver); (d) controlling, ameliorating, and/or preventing the infiltration of inflammatory cells (e.g., NK cells, cytotoxic T cells, neutrophils, monocytes, macrophages) in tissues and/or organs (infected or non-infected) in vitro and/or in vivo, such as reducing at least about 10% (including for example at least about any of 20%, 30%, 40%, 50%, 60%, 70%, 80%, 90%, or 100%) inflammatory cell infiltration; (e) controlling, ameliorating and/or preventing inflammation in infected or non-infected tissue and/or organ, systemic inflammation, and/or cytokine storm, e.g., changing the levels of inflammatory markers such as IL-6, IL-8, IL-10, IL1B, IL-12, IL-15, IL-17, CCL2, IL-1α, IL-2, IL-5, IL-9, CCL4, M-CSF, MCP-1, GCSF, MIP1A, C-reactive protein (CRP), TNFα, TNFβ, IFNγ, IP10, MCP1, and serum amyloid A1 (SAA1), such as downregulating at least about 10% (including for example at least about any of 20%, 30%, 40%, 50%, 60%, 70%, 80%, 90%, or 100%), or down-regulating (e.g., downregulating at least about any of 5%, 10%, 20%, 30%, 40%, 50%, 60%, 70%, 80%, 90%, or more) pro-inflammatory pathways such as TLR4 signaling; (f) promoting tissue and/or organ regeneration, such as changing the levels of regeneration markers such as angiopoietin-2 (ANGPT2), FGF-b, Platelet-derived growth factor AA (PDGF-AA), regenerating islet-derived protein 3 alpha (Reg3A), and PDGF-BB (e.g., upregulating at least about 10% (including for example at least about any of 20%, 30%, 40%, 50%, 60%, 70%, 80%, 90%, or 100%)); (g) protecting tissue and/or organ (e.g., lung, heart, kidney, liver) from adverse effects (e.g., injury) triggered by additional therapy, such as antiviral drugs; (h) decreasing acute respiratory distress syndrome (ARDS) score for viral infection associated with respiratory system (e.g., lung); (i) controlling, ameliorating, and/or preventing sepsis, SIRS, septic shock, and/or MODS; (j) reducing mortality rate associated with virus infection, and/or preventing death, such as reducing at least about 10% (including for example at least about any of 20%, 30%, 40%, 50%, 60%, 70%, 80%, 90%, or 100%) death rate; (k) decreasing Acute Physiology And Chronic Health Evaluation II (APACHE II) score or KNAUS score (for MODS) in an individual; (l) improving organ function test scores (e.g., lung function test score); (m) treating or preventing metabolic disease, fatty liver, hepatitis, sepsis, MODS, neurological disorder, and pancreatitis associated with viral infection; (n) increasing point (e.g., greater than or equal to 2-point increase) in the National Institute of Allergy and Infectious Diseases (NIAID) 8-point ordinal scale; (o) reducing length of hospital stay (e.g., reducing at least about any of 1, 2, 3, 4, 5, 10, 20, 30, 60, 90, 120, 180, or more days of hospital stay); (p) increasing alive and respiratory failure free days (e.g., increasing at least about any of 1, 2, 3, 4, 5, 10, 20, 30, 60, 90, 120, 180, or more days); (q) controlling, ameliorating, and/or preventing progression to severe/critical disease (e.g., reducing or preventing at least about any of 5%, 10%, 20%, 30%, 40%, 50%, 60%, 70%, 80%, 90%, or more severe progression); (r) controlling, reducing, and/or preventing occurrence of any new infections (e.g., reducing or preventing at least about any of 5%, 10%, 20%, 30%, 40%, 50%, 60%, 70%, 80%, 90%, or more new infections); (s) controlling, ameliorating, and/or preventing endothelial (e.g., pulmonary endothelial) dysfunction, injury, or death (e.g., reducing or preventing at least about any of 5%, 10%, 20%, 30%, 40%, 50%, 60%, 70%, 80%, 90%, or more endothelial dysfunction, injury, or death); (t) controlling, ameliorating, and/or preventing (e.g., reducing or preventing at least about any of 5%, 10%, 20%, 30%, 40%, 50%, 60%, 70%, 80%, 90%, or more) damage and/or degradation of EGX, endothelial cell surface proteins, and/or adherens junctions between endothelial cells, such as by down-regulating (e.g., down-regulating at least about any of 5%, 10%, 20%, 30%, 40%, 50%, 60%, 70%, 80%, 90%, or more) extracellular proteinase (e.g., MMP) expression and/or up-regulating (e.g., up-regulating at least about any of 5%, 10%, 20%, 30%, 40%, 50%, 60%, 70%, 80%, 90%, or more) extracellular matrix protein expression (e.g., Tenascin C (Tnc), collagen, type I, alpha 1 (COL1a1), collagen, type VI, alpha 3 (Col6a3), and collagen, type I, alpha 2 (Col1a2)); (u) controlling, ameliorating, and/or preventing (e.g., reducing or preventing at least about any of 5%, 10%, 20%, 30%, 40%, 50%, 60%, 70%, 80%, 90%, or more) protein leakage; (v) promoting regeneration of EGX and/or endothelial (e.g., pulmonary endothelial) cells, such as increasing at least about any of 5%, 10%, 20%, 30%, 40%, 50%, 60%, 70%, 80%, 90%, or more functional EGX and/or endothelial cells; (w) reducing (e.g., at least about any of 5%, 10%, 20%, 30%, 40%, 50%, 60%, 70%, 80%, 90%, or more) viral load in infected tissue and/or organ; and (x) reducing or preventing (e.g., at least about any of 5%, 10%, 20%, 30%, 40%, 50%, 60%, 70%, 80%, 90%, or more) organ (e.g., lung) collagen deposition. In some embodiments, the virus-induced organ injury or failure is virus-induced lung injury or failure, such as pulmonary fibrosis, pneumonia, ALI, SARS, MERS, COVID-19, H1N1 swine flu, H5N1 bird flu, or ARDS. In some embodiments, the virus-induced organ injury or failure is virus-induced sepsis, septic shock, or MODS. In some embodiments, the virus-induced organ injury or failure is caused by a virus of any one of the Orthomyxoviridae, Filoviridae, Flaviviridae, Coronaviridae, and Poxviridae families. In some embodiments, the virus is an Orthomyxoviridae virus selected from the group consisting of Influenza A virus, Influenza B virus, Influenza C virus, and any subtype or reassortant thereof. In some embodiments, the virus is an Influenza A virus or any subtype or reassortant thereof, such as H1N1 or H5N1. In some embodiments, the virus is a Coronaviridae virus selected from the group consisting of alpha coronaviruses 229E (HCoV-229E), New Haven coronavirus NL63 (HCoV-NL63), beta coronaviruses OC43 (HCoV-OC43), coronavirus HKU1 (HCoV-HKU1), Severe Acute Respiratory Syndrome coronavirus (SARS-CoV), Middle East Respiratory Syndrome coronavirus (MERS-CoV), and Severe Acute Respiratory Syndrome Coronavirus 2 (SARS-CoV-2). In some embodiments, the virus is a Filoviridae virus selected from Ebola virus (EBOV) and Marburg virus (MARV). In some embodiments, the virus is a Flaviviridae virus selected from the group consisting of Zika virus (ZIKV), West Nile virus (WNV), Dengue virus (DENV), and Yellow Fever virus (YFV). In some embodiments, the method further comprises administering to the individual an effective amount of another therapeutic agent. In some embodiments, the other therapeutic agent is selected from the group consisting of a corticosteroid, an anti-inflammatory signal transduction modulator, a 02-adrenoreceptor agonist bronchodilator, an anticholinergic, a mucolytic agent, an antiviral agent, an anti-fibrotic agent, hypertonic saline, an antibody, a vaccine, or mixtures thereof. In some embodiments, the antiviral agent is selected from the group consisting of remdesivir, lopinavir/ritonavir (Kaletra®, e.g., tablet), IFN-α (e.g., IFN-α2a, IFN-α2b, via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir, zanamivir, peramivir, amantadine, rimantadine, favipiravir, laninamivir, ribavirin, umifenovir (Arbidol®), and any combinations thereof. In some embodiments, the other therapeutic agent is selected from the group consisting of remdesivir, lopinavir/ritonavir (Kaletra®, e.g., tablet), IFN-α (e.g., IFN-α2a or IFN-α2b, via inhalation), favipiravir, lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, and any combinations thereof, and the virus-induced organ injury or failure is induced by SARS-CoV-2. In some embodiments, the other therapeutic agent is remdesivir and the virus-induced organ injury or failure is induced by SARS-CoV-2. In some embodiments, the other therapeutic agent is lopinavir/ritonavir (Kaletra®, e.g., tablet) and IFN-α (e.g., via inhalation), and the virus-induced organ injury or failure is induced by SARS-CoV-2. In some embodiments, the other therapeutic agent is selected from the group consisting of oseltamivir, zanamivir, peramivir, favipiravir, umifenovir (Arbidol®), teicoplanin derivatives, benzo-heterocyclic amine derivative, pyrimidine, baloxavir marboxil, lopinavir/ritonavir (Kaletra®, e.g., tablet), IFN-α (e.g., via inhalation), and any combinations thereof, and the virus-induced organ injury or failure is induced by H1N1 or H5N1. In some embodiments, the other therapeutic agent is lopinavir/ritonavir (Kaletra®, e.g., tablet) and IFN-α (e.g., via inhalation), and the virus-induced organ injury or failure is induced by H1N1 or H5N1. In some embodiments, the anti-fibrotic agent is selected from the group consisting of nintedanib, pirfenidone, and N-Acetylcysteine (NAC). In some embodiments, the IL-22 dimer is administered simultaneously with or subsequent to the other therapeutic agent. In some embodiments, the IL-22 dimer comprises Formula I: M1-L-M2; wherein Ml is a first IL-22 monomer, M2 is a second IL-22 monomer, and L is a linking moiety connecting the first IL-22 monomer and the second IL-22 monomer and disposed therebetween. In some embodiments, the linking moiety L is a short polypeptide comprising about 3 to about 50 amino acids (such as any one of SEQ ID NOs: 1-20 and 32). In some embodiments, IL-22 dimer comprises (or consists essentially of, or consists of) in SEQ ID NO: 28. In some embodiments, the linking moiety L is a polypeptide of Formula II: —Z—Y—Z—; wherein Y is a carrier protein (e.g., albumin such as human albumin, Fc fragment); Z is nothing, or a short peptide comprising about 1 to about 50 amino acids (such as any one of SEQ ID NOs: 1-20 and 32); and “-” is a chemical bond or a covalent bond (e.g., peptide bond). In some embodiments, the IL-22 dimer comprises two monomeric subunits, wherein each monomeric subunit comprises an IL-22 monomer and a dimerization domain. In some embodiments, the IL-22 monomer is connected to the dimerization domain via an optional linker. In some embodiments, the linker comprises the sequence of any one of SEQ ID NOs: 1-20 and 32. In some embodiments, the linker is about 6 to about 30 amino acids in length. In some embodiments, the linker comprises the sequence of SEQ ID NO: 1 or 10. In some embodiments, the dimerization domain comprises at least two cysteines capable of forming intermolecular disulfide bonds. In some embodiments, the dimerization domain comprises at least a portion of an Fc fragment. In some embodiments, the Fc fragment comprises CH2 and CH3 domains. In some embodiments, the Fc fragment comprises the sequence of SEQ ID NO: 22 or 23. In some embodiments, the IL-22 monomer comprises the sequence of SEQ ID NO: 21. In some embodiments, the IL-22 monomer is N-terminal to the dimerization domain. In some embodiments, the IL-22 monomer is C-terminal to the dimerization domain. In some embodiments, each monomeric subunit comprises the sequence of any of SEQ ID NOs: 24-27 (such as SEQ ID NO: 24). In some embodiments, the IL-22 dimer is administered intravenously, intrapulmonarily, or via inhalation or insufflation. In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered at least once a week. In some embodiments, the IL-22 dimer is administered on day 1 and day 6 of a 10-day treatment cycle, or day 1 and day 8 of a 14-day treatment cycle. In some embodiments, the individual (e.g., human) is at least about 55 years old (e.g., at least about any of 60, 65, 70, 75, 80, 85, 90 years old, or older). In some embodiments, the method further comprises selecting the individual based on that the individual is at least about 55 years old (e.g., at least about any of 60, 65, 70, 75, 80, 85, 90 years old, or older).

Thus in some embodiments, there is provided a method of preventing or treating a virus-induced organ injury or failure (e.g., necrosis, lung injury or failure such as pulmonary fibrosis, pneumonia, ALI, SARS, MERS, COVID-19, H1N1 swine flu, H5N1 bird flu, or ARDS, sepsis, septic shock, MODS, death) in an individual (e.g., human, such as a human of at least about 55 years old), comprising administering to the individual an effective amount of an IL-22 dimer. In some embodiments, there is provided a method of preventing or treating a virus-induced organ injury or failure (e.g., necrosis, lung injury or failure such as pulmonary fibrosis, pneumonia, ALI, SARS, MERS, COVID-19, H1N1 swine flu, H5N1 bird flu, or ARDS, sepsis, septic shock, MODS, death) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer and a dimerization domain. In some embodiments, the IL-22 monomer is connected to the dimerization domain via an optional linker. In some embodiments, the linker comprises the sequence of any one of SEQ ID NOs: 1-20 and 32. In some embodiments, the linker is about 6 to about 30 amino acids in length. In some embodiments, the linker comprises the sequence of SEQ ID NO: 1 or 10. In some embodiments, the dimerization domain comprises at least two cysteines capable of forming intermolecular disulfide bonds. In some embodiments, the dimerization domain comprises at least a portion of an Fc fragment. In some embodiments, the Fc fragment comprises CH2 and CH3 domains. In some embodiments, the Fc fragment comprises the sequence of SEQ ID NO: 22 or 23. In some embodiments, the IL-22 monomer comprises the sequence of SEQ ID NO: 21. In some embodiments, the IL-22 monomer is N-terminal to the dimerization domain. In some embodiments, the IL-22 monomer is C-terminal to the dimerization domain. In some embodiments, each monomeric subunit comprises the sequence of any of SEQ ID NOs: 24-27 (such as SEQ ID NO: 24). Thus in some embodiments, there is provided a method of preventing or treating a virus-induced organ injury or failure (e.g., necrosis, lung injury or failure such as pulmonary fibrosis, pneumonia, ALI, SARS, MERS, COVID-19, H1N1 swine flu, H5N1 bird flu, or ARDS, sepsis, septic shock, MODS, death) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the virus belongs to any one of the Orthomyxoviridae, Filoviridae, Flaviviridae, Coronaviridae, and Poxviridae families. In some embodiments, the virus is SARS-CoV, MERS-CoV, SARS-CoV-2, H1N1, or H5N1. In some embodiments, the method comprises reducing ARDS score, APACHE II score, and/or KNAUS score. In some embodiments, the method comprises improving organ (e.g., lung, heart, liver, kidney) function test score. In some embodiments, the method comprises increasing point of NIAID 8-point ordinal scale. In some embodiments, the virus-induced organ injury or failure comprises endothelial cell injury, dysfunction, or death. In some embodiments, endothelial dysfunction comprises EGX degradation. In some embodiments, the method comprises one or more of: i) reducing and/or preventing endothelial cell injury, dysfunction, or death, and/or EGX degradation/damage; ii) regenerating functional endothelial (e.g., pulmonary endothelial) cells and/or EGX; iii) reducing and/or preventing inflammatory cell (e.g., NK cell, CTL, neutrophil, monocyte, macrophage) infiltration; iv) reducing viral load in infected tissue and/or organ; or v) reducing and/or preventing organ (e.g., lung) collagen deposition. In some embodiments, the method further comprises selecting the individual based on that the individual is at least about 55 years old (e.g., at least about any of 60, 65, 70, 75, 80, 85, 90 years old, or older). In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., remdesivir, oseltamivir, zanamivir, peramivir, lopinavir/ritonavir (Kaletra®), and/or IFN-α).

Thus in some embodiments, there is provided a method of preventing or treating a SARS-CoV-induced lung injury or failure (e.g., pulmonary fibrosis, pneumonia, ALI, ARDS, SARS) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of preventing or treating a SARS-CoV-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of preventing or treating a SARS-CoV-induced lung injury or failure (e.g., pulmonary fibrosis, pneumonia, ALI, ARDS, SARS) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of preventing or treating a SARS-CoV-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., remdesivir, lopinavir/ritonavir (Kaletra®, e.g., tablet), and/or IFN-α (e.g., via inhalation)). In some embodiments, the method comprises reducing ARDS score, APACHE II score, and/or KNAUS score. In some embodiments, the method comprises improving organ (e.g., lung, heart, liver, kidney) function test score. In some embodiments, the method comprises increasing point of NIAID 8-point ordinal scale. In some embodiments, the method further comprises selecting the individual based on that the individual is at least about 55 years old (e.g., at least about any of 60, 65, 70, 75, 80, 85, 90 years old, or older).

In some embodiments, there is provided a method of preventing or treating a MERS-CoV-induced lung injury or failure (e.g., pulmonary fibrosis, pneumonia, ALI, ARDS, MERS) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of preventing or treating a MERS-CoV-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of preventing or treating a MERS-CoV-induced lung injury or failure (e.g., pulmonary fibrosis, pneumonia, ALI, ARDS, MERS) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of preventing or treating a MERS-CoV-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., remdesivir, lopinavir/ritonavir (Kaletra®, e.g., tablet), and/or IFN-α (e.g., via inhalation)). In some embodiments, the method comprises reducing ARDS score, APACHE II score, and/or KNAUS score. In some embodiments, the method comprises improving organ (e.g., lung, heart, liver, kidney) function test score. In some embodiments, the method comprises increasing point of NIAID 8-point ordinal scale. In some embodiments, the method further comprises selecting the individual based on that the individual is at least about 55 years old (e.g., at least about any of 60, 65, 70, 75, 80, 85, 90 years old, or older).

In some embodiments, there is provided a method of preventing or treating a SARS-CoV-2-induced lung injury or failure (e.g., pulmonary fibrosis, pneumonia, ALI, ARDS, COVID-19) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of preventing or treating a SARS-CoV-2-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of preventing or treating a SARS-CoV-2-induced lung injury or failure (e.g., pulmonary fibrosis, pneumonia, ALI, ARDS, COVID-19) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of preventing or treating a SARS-CoV-2-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of ameliorating pulmonary fibrosis due to SARS-CoV-2 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between.). In some embodiments, there is provided a method of ameliorating pulmonary fibrosis due to SARS-CoV-2 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., remdesivir, lopinavir/ritonavir (Kaletra®, e.g., tablet), and/or IFN-α (e.g., via inhalation)). In some embodiments, the method comprises reducing ARDS score, APACHE II score, and/or KNAUS score. In some embodiments, the method comprises improving organ (e.g., lung, heart, liver, kidney) function test score. In some embodiments, the method comprises increasing point of MAID 8-point ordinal scale. In some embodiments, the method comprises one or more of: i) reducing and/or preventing endothelial cell injury, dysfunction, or death, and/or EGX degradation/damage; ii) regenerating functional endothelial (e.g., pulmonary endothelial) cells and/or EGX; iii) reducing and/or preventing inflammatory cell (e.g., NK cell, CTL, neutrophil, monocyte, macrophage) infiltration; iv) reducing viral load in infected tissue and/or organ; or v) reducing and/or preventing organ (e.g., lung) collagen deposition. In some embodiments, the method further comprises selecting the individual based on that the individual is at least about 55 years old (e.g., at least about any of 60, 65, 70, 75, 80, 85, 90 years old, or older).

In some embodiments, there is provided a method of preventing or treating an H1N1-induced lung injury or failure (e.g., pulmonary fibrosis, pneumonia, ALI, ARDS, H1N1 swine flu) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of preventing or treating an H1N1-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of preventing or treating an H1N1-induced lung injury or failure (e.g., pulmonary fibrosis, pneumonia, ALI, ARDS, H1N1 swine flu) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of preventing or treating an H1N1-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of ameliorating pulmonary fibrosis due to H1N1 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between.). In some embodiments, there is provided a method of ameliorating pulmonary fibrosis due to H1N1 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., oseltamivir, zanamivir, or peramivir, lopinavir/ritonavir (Kaletra®, e.g., tablet), and/or IFN-α (e.g., via inhalation)). In some embodiments, the method comprises reducing ARDS score, APACHE II score, and/or KNAUS score. In some embodiments, the method comprises improving organ (e.g., lung, heart, liver, kidney) function test score. In some embodiments, the method comprises increasing point of MAID 8-point ordinal scale. In some embodiments, the method comprises one or more of: i) reducing and/or preventing endothelial cell injury, dysfunction, or death, and/or EGX degradation/damage; ii) regenerating functional endothelial (e.g., pulmonary endothelial) cells and/or EGX; iii) reducing and/or preventing inflammatory cell (e.g., NK cell, CTL, neutrophil, monocyte, macrophage) infiltration; iv) reducing viral load in infected tissue and/or organ; or v) reducing and/or preventing organ (e.g., lung) collagen deposition. In some embodiments, the method further comprises selecting the individual based on that the individual is at least about 55 years old (e.g., at least about any of 60, 65, 70, 75, 80, 85, 90 years old, or older).

In some embodiments, there is provided a method of preventing or treating an H5N1-induced lung injury or failure (e.g., pulmonary fibrosis, pneumonia, ALI, ARDS, H5N1 bird flu) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of preventing or treating an H5N1-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of preventing or treating an H5N1-induced lung injury or failure (e.g., pulmonary fibrosis, pneumonia, ALI, ARDS, H5N1 bird flu) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of preventing or treating an H5N1-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., oseltamivir, zanamivir, or peramivir, lopinavir/ritonavir (Kaletra®, e.g., tablet), and/or IFN-α (e.g., via inhalation)). In some embodiments, the method comprises reducing ARDS score, APACHE II score, and/or KNAUS score. In some embodiments, the method comprises improving organ (e.g., lung, heart, liver, kidney) function test score. In some embodiments, the method comprises increasing point of NIAID 8-point ordinal scale. In some embodiments, the method further comprises selecting the individual based on that the individual is at least about 55 years old (e.g., at least about any of 60, 65, 70, 75, 80, 85, 90 years old, or older).

In some embodiments, there is provided a method of protecting an organ (e.g., lung, heart, liver, kidney) from virus-induced organ injury or failure (e.g., necrosis, lung injury or failure such as pulmonary fibrosis, pneumonia, ALI, SARS, MERS, COVID-19, H1N1 swine flu, H5N1 bird flu, or ARDS, sepsis, septic shock, MODS) in an individual (e.g., human, such as a human of at least about 55 years old), comprising administering to the individual an effective amount of an IL-22 dimer. In some embodiments, there is provided a method of protecting an organ (e.g., lung, heart, liver, kidney) from virus-induced organ injury or failure (e.g., necrosis, lung injury or failure such as pulmonary fibrosis, pneumonia, ALI, SARS, MERS, COVID-19, H1N1 swine flu, H5N1 bird flu, or ARDS, sepsis, septic shock, MODS) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer and a dimerization domain. In some embodiments, the IL-22 monomer is connected to the dimerization domain via an optional linker. In some embodiments, the linker comprises the sequence of any one of SEQ ID NOs: 1-20 and 32. In some embodiments, the linker is about 6 to about 30 amino acids in length. In some embodiments, the linker comprises the sequence of SEQ ID NO: 1 or 10. In some embodiments, the dimerization domain comprises at least two cysteines capable of forming intermolecular disulfide bonds. In some embodiments, the dimerization domain comprises at least a portion of an Fc fragment. In some embodiments, the Fc fragment comprises CH2 and CH3 domains. In some embodiments, the Fc fragment comprises the sequence of SEQ ID NO: 22 or 23. In some embodiments, the IL-22 monomer comprises the sequence of SEQ ID NO: 21. In some embodiments, the IL-22 monomer is N-terminal to the dimerization domain. In some embodiments, the IL-22 monomer is C-terminal to the dimerization domain. In some embodiments, each monomeric subunit comprises the sequence of any of SEQ ID NOs: 24-27 (such as SEQ ID NO: 24). Thus in some embodiments, there is provided a method of protecting an organ (e.g., lung, heart, liver, kidney) from virus-induced organ injury or failure (e.g., necrosis, lung injury or failure such as pulmonary fibrosis, pneumonia, ALI, SARS, MERS, COVID-19, H1N1 swine flu, H5N1 bird flu, or ARDS, sepsis, septic shock, MODS) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the virus belongs to any one of the Orthomyxoviridae, Filoviridae, Flaviviridae, Coronaviridae, and Poxviridae families. In some embodiments, the virus is SARS-CoV, MERS-CoV, SARS-CoV-2, H1N1, or H5N1. In some embodiments, the method comprises reducing ARDS score, APACHE II score, and/or KNAUS score. In some embodiments, the method comprises improving organ (e.g., lung, heart, liver, kidney) function test score. In some embodiments, the method comprises increasing point of NIAID 8-point ordinal scale. In some embodiments, virus-induced organ injury or failure or MODS comprises endothelial cell injury, dysfunction, or death. In some embodiments, endothelial dysfunction comprises EGX degradation. In some embodiments, the method comprises one or more of: i) reducing and/or preventing endothelial cell injury, dysfunction, or death, and/or EGX degradation/damage; ii) regenerating functional endothelial (e.g., pulmonary endothelial) cells and/or EGX; iii) reducing and/or preventing inflammatory cell (e.g., NK cell, CTL, neutrophil, monocyte, macrophage) infiltration; iv) reducing viral load in infected tissue and/or organ; or v) reducing and/or preventing organ (e.g., lung) collagen deposition. In some embodiments, the method further comprises selecting the individual based on that the individual is at least about 55 years old (e.g., at least about any of 60, 65, 70, 75, 80, 85, 90 years old, or older). In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., remdesivir, oseltamivir, zanamivir, peramivir, lopinavir/ritonavir (Kaletra®), and/or IFN-α).

Thus in some embodiments, there is provided a method of protecting lung from SARS-CoV-induced lung injury or failure (e.g., pulmonary fibrosis, pneumonia, ALI, ARDS, SARS) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of protecting an organ (e.g., lung, heart, liver, kidney) from SARS-CoV-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of protecting lung from SARS-CoV-induced lung injury or failure (e.g., pulmonary fibrosis, pneumonia, ALI, ARDS, SARS) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of protecting an organ (e.g., lung, heart, liver, kidney) from SARS-CoV-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., remdesivir, lopinavir/ritonavir (Kaletra®, e.g., tablet), and/or IFN-α (e.g., via inhalation)). In some embodiments, the method comprises reducing ARDS score, APACHE II score, and/or KNAUS score. In some embodiments, the method comprises improving organ (e.g., lung, heart, liver, kidney) function test score. In some embodiments, the method comprises increasing point of NIAID 8-point ordinal scale. In some embodiments, the method further comprises selecting the individual based on that the individual is at least about 55 years old (e.g., at least about any of 60, 65, 70, 75, 80, 85, 90 years old, or older).

In some embodiments, there is provided a method of protecting lung from a MERS-CoV-induced lung injury or failure (e.g., pulmonary fibrosis, pneumonia, ALI, ARDS, MERS) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of protecting an organ (e.g., lung, heart, liver, kidney) from a MERS-CoV-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of protecting lung from a MERS-CoV-induced lung injury or failure (e.g., pulmonary fibrosis, pneumonia, ALI, ARDS, MERS) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of protecting an organ (e.g., lung, heart, liver, kidney) from a MERS-CoV-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., remdesivir, lopinavir/ritonavir (Kaletra®, e.g., tablet), and/or IFN-α (e.g., via inhalation)). In some embodiments, the method comprises reducing ARDS score, APACHE II score, and/or KNAUS score. In some embodiments, the method comprises improving organ (e.g., lung, heart, liver, kidney) function test score. In some embodiments, the method comprises increasing point of NIAID 8-point ordinal scale. In some embodiments, the method further comprises selecting the individual based on that the individual is at least about 55 years old (e.g., at least about any of 60, 65, 70, 75, 80, 85, 90 years old, or older).

In some embodiments, there is provided a method of protecting lung from a SARS-CoV-2-induced lung injury or failure (e.g., pulmonary fibrosis, pneumonia, ALI, ARDS, COVID-19) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of protecting an organ (e.g., lung, heart, liver, kidney) from a SARS-CoV-2-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of protecting lung from a SARS-CoV-2-induced lung injury or failure (e.g., pulmonary fibrosis, pneumonia, ALI, ARDS, COVID-19) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of protecting an organ (e.g., lung, heart, liver, kidney) from a SARS-CoV-2-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., remdesivir, lopinavir/ritonavir (Kaletra®, e.g., tablet), and/or IFN-α (e.g., via inhalation)). In some embodiments, the method comprises reducing ARDS score, APACHE II score, and/or KNAUS score. In some embodiments, the method comprises improving organ (e.g., lung, heart, liver, kidney) function test score. In some embodiments, the method comprises increasing point of MAID 8-point ordinal scale. In some embodiments, the method comprises one or more of: i) reducing and/or preventing endothelial cell injury, dysfunction, or death, and/or EGX degradation/damage; ii) regenerating functional endothelial (e.g., pulmonary endothelial) cells and/or EGX; iii) reducing and/or preventing inflammatory cell (e.g., NK cell, CTL, neutrophil, monocyte, macrophage) infiltration; iv) reducing viral load in infected tissue and/or organ; or v) reducing and/or preventing organ (e.g., lung) collagen deposition. In some embodiments, the method further comprises selecting the individual based on that the individual is at least about 55 years old (e.g., at least about any of 60, 65, 70, 75, 80, 85, 90 years old, or older).

In some embodiments, there is provided a method of protecting lung from an H1N1-induced lung injury or failure (e.g., pulmonary fibrosis, pneumonia, ALI, ARDS, H1N1 swine flu) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of protecting an organ (e.g., lung, heart, liver, kidney) from an H1N1-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of protecting lung from an H1N1-induced lung injury or failure (e.g., pulmonary fibrosis, pneumonia, ALI, ARDS, H1N1 swine flu) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of protecting an organ (e.g., lung, heart, liver, kidney) from an H1N1-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., oseltamivir, zanamivir, peramivir, lopinavir/ritonavir (Kaletra®, e.g., tablet), and/or IFN-α (e.g., via inhalation)). In some embodiments, the method comprises reducing ARDS score, APACHE II score, and/or KNAUS score. In some embodiments, the method comprises improving organ (e.g., lung, heart, liver, kidney) function test score. In some embodiments, the method comprises increasing point of NIAID 8-point ordinal scale. In some embodiments, the method comprises one or more of: i) reducing and/or preventing endothelial cell injury, dysfunction, or death, and/or EGX degradation/damage; ii) regenerating functional endothelial (e.g., pulmonary endothelial) cells and/or EGX; iii) reducing and/or preventing inflammatory cell (e.g., NK cell, CTL, neutrophil, monocyte, macrophage) infiltration; iv) reducing viral load in infected tissue and/or organ; or v) reducing and/or preventing organ (e.g., lung) collagen deposition. In some embodiments, the method further comprises selecting the individual based on that the individual is at least about 55 years old (e.g., at least about any of 60, 65, 70, 75, 80, 85, 90 years old, or older).

In some embodiments, there is provided a method of protecting lung from an H5N1-induced lung injury or failure (e.g., pulmonary fibrosis, pneumonia, ALI, ARDS, H5N1 bird flu) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of protecting an organ (e.g., lung, heart, liver, kidney) from an H5N1-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of preventing or treating an H5N1-induced lung injury or failure (e.g., pulmonary fibrosis, pneumonia, ALI, ARDS, H5N1 bird flu) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of preventing or treating an H5N1-induced MODS in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., oseltamivir, zanamivir, peramivir, lopinavir/ritonavir (Kaletra®, e.g., tablet), and/or IFN-α (e.g., via inhalation)). In some embodiments, the method comprises reducing ARDS score, APACHE II score, and/or KNAUS score. In some embodiments, the method comprises improving organ (e.g., lung, heart, liver, kidney) function test score. In some embodiments, the method comprises increasing point of NIAID 8-point ordinal scale. In some embodiments, the method further comprises selecting the individual based on that the individual is at least about 55 years old (e.g., at least about any of 60, 65, 70, 75, 80, 85, 90 years old, or older).

In some embodiments, there is provided a method of reducing inflammation (e.g., viral activity, infiltration of inflammatory cells (e.g., CTL, NK cell, neutrophil, monocyte, macrophage), inflammatory biomarkers, cytokine storm, SIRS, sepsis, septic shock) due to virus infection in an individual (e.g., human, such as a human of at least about 55 years old), comprising administering to the individual an effective amount of an IL-22 dimer. In some embodiments, there is provided a method of reducing inflammation (e.g., viral activity, infiltration of inflammatory cells (e.g., CTL, NK cell, neutrophil, monocyte, macrophage), inflammatory biomarkers, cytokine storm, SIRS, sepsis, septic shock) due to virus infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer and a dimerization domain. In some embodiments, the IL-22 monomer is connected to the dimerization domain via an optional linker. In some embodiments, the linker comprises the sequence of any one of SEQ ID NOs: 1-20 and 32. In some embodiments, the linker is about 6 to about 30 amino acids in length. In some embodiments, the linker comprises the sequence of SEQ ID NO: 1 or 10. In some embodiments, the dimerization domain comprises at least two cysteines capable of forming intermolecular disulfide bonds. In some embodiments, the dimerization domain comprises at least a portion of an Fc fragment. In some embodiments, the Fc fragment comprises CH2 and CH3 domains. In some embodiments, the Fc fragment comprises the sequence of SEQ ID NO: 22 or 23. In some embodiments, the IL-22 monomer comprises the sequence of SEQ ID NO: 21. In some embodiments, the IL-22 monomer is N-terminal to the dimerization domain. In some embodiments, the IL-22 monomer is C-terminal to the dimerization domain. In some embodiments, each monomeric subunit comprises the sequence of any of SEQ ID NOs: 24-27 (such as SEQ ID NO: 24). Thus in some embodiments, there is provided a method of reducing inflammation (e.g., viral activity, infiltration of inflammatory cells (e.g., CTL, NK cell, neutrophil, monocyte, macrophage), inflammatory biomarkers, cytokine storm, SIRS, sepsis, septic shock) due to virus infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about g/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the virus belongs to any one of the Orthomyxoviridae, Filoviridae, Flaviviridae, Coronaviridae, and Poxviridae families. In some embodiments, the virus is SARS-CoV, MERS-CoV, SARS-CoV-2, H1N1, or H5N1. In some embodiments, the method comprises reducing inflammatory biomarkers such as IL-6, IL-8, IL-10, IL1B, IL-12, IL-15, IL-17, CCL2, IL-1α, IL-2, IL-5, IL-9, CCL4, M-CSF, MCP-1, GCSF, MIP1A, CRP, TNFα, TNFβ, IFNγ, IP10, MCP1, and SAA1. In some embodiments, the method comprises reducing APACHE II score and/or KNAUS score. In some embodiments, the method comprises increasing point of MAID 8-point ordinal scale. In some embodiments, the method comprises one or more of: i) reducing viral load in infected tissue and/or organ; or ii) reducing and/or preventing organ (e.g., lung) collagen deposition. In some embodiments, the method further comprises selecting the individual based on that the individual is at least about 55 years old (e.g., at least about any of 60, 65, 70, 75, 80, 85, 90 years old, or older). In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., remdesivir, oseltamivir, zanamivir, peramivir, lopinavir/ritonavir (Kaletra®), and/or IFN-α).

Thus in some embodiments, there is provided a method of reducing inflammation (e.g., viral activity, infiltration of inflammatory cells (e.g., CTL, NK cell, neutrophil, monocyte, macrophage), inflammatory biomarkers, cytokine storm, SIRS, sepsis, septic shock) due to SARS-CoV infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of reducing cytokine storm due to SARS-CoV infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of reducing inflammation (e.g., viral activity, infiltration of inflammatory cells (e.g., CTL, NK cell, neutrophil, monocyte, macrophage), inflammatory biomarkers, cytokine storm, SIRS, sepsis, septic shock) due to SARS-CoV infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of reducing cytokine storm due to SARS-CoV infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., remdesivir, lopinavir/ritonavir (Kaletra®, e.g., tablet), and/or IFN-α (e.g., via inhalation)). In some embodiments, the method comprises reducing inflammatory biomarkers such as IL-6, IL-8, IL-10, IL1B, IL-12, IL-15, IL-17, CCL2, IL-1α, IL-2, IL-5, IL-9, CCL4, M-CSF, MCP-1, GCSF, MIP1A, CRP, TNFα, TNFβ, IFNγ, IP10, MCP1, and SAA1. In some embodiments, the method comprises reducing APACHE II score and/or KNAUS score. In some embodiments, the method comprises increasing point of MAID 8-point ordinal scale. In some embodiments, the method comprises one or more of: i) reducing viral load in infected tissue and/or organ; or ii) reducing and/or preventing organ (e.g., lung) collagen deposition. In some embodiments, the method further comprises selecting the individual based on that the individual is at least about 55 years old (e.g., at least about any of 60, 65, 70, 75, 80, 85, 90 years old, or older).

In some embodiments, there is provided a method of reducing inflammation (e.g., viral activity, infiltration of inflammatory cells (e.g., CTL, NK cell, neutrophil, monocyte, macrophage), inflammatory biomarkers, cytokine storm, SIRS, sepsis, septic shock) due to MERS-CoV infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of reducing cytokine storm due to MERS-CoV infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of reducing inflammation (e.g., viral activity, infiltration of inflammatory cells (e.g., CTL, NK cell, neutrophil, monocyte, macrophage), inflammatory biomarkers, cytokine storm, SIRS, sepsis, septic shock) due to MERS-CoV infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of reducing cytokine storm due to MERS-CoV infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., remdesivir, lopinavir/ritonavir (Kaletra®, e.g., tablet), and/or IFN-α (e.g., via inhalation)). In some embodiments, the method comprises reducing inflammatory biomarkers such as IL-6, IL-8, IL-10, IL1B, IL-12, IL-15, IL-17, CCL2, IL-1α, IL-2, IL-5, IL-9, CCL4, M-CSF, MCP-1, GCSF, MIP1A, CRP, TNFα, TNFβ, IFNγ, IP10, MCP1, and SAA1. In some embodiments, the method comprises reducing APACHE II score and/or KNAUS score. In some embodiments, the method comprises increasing point of MAID 8-point ordinal scale. In some embodiments, the method comprises one or more of: i) reducing viral load in infected tissue and/or organ; or ii) reducing and/or preventing organ (e.g., lung) collagen deposition. In some embodiments, the method further comprises selecting the individual based on that the individual is at least about 55 years old (e.g., at least about any of 60, 65, 70, 75, 80, 85, 90 years old, or older).

In some embodiments, there is provided a method of reducing inflammation (e.g., viral activity, infiltration of inflammatory cells (e.g., CTL, NK cell, neutrophil, monocyte, macrophage), inflammatory biomarkers, cytokine storm, SIRS, sepsis, septic shock) due to SARS-CoV-2 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of reducing cytokine storm due to SARS-CoV-2 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of reducing inflammation (e.g., viral activity, infiltration of inflammatory cells (e.g., CTL, NK cell, neutrophil, monocyte, macrophage), inflammatory biomarkers, cytokine storm, SIRS, sepsis) due to SARS-CoV-2 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of reducing cytokine storm due to SARS-CoV-2 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of reducing viral load in SARS-CoV-2 infected organ (e.g., lung) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of reducing viral load in SARS-CoV-2 infected organ (e.g., lung) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of preventing SARS-CoV-2 infection (e.g., lung infection) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of preventing SARS-CoV-2 infection (e.g., lung infection) in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., remdesivir, lopinavir/ritonavir (Kaletra®, e.g., tablet), and/or IFN-α (e.g., via inhalation)). In some embodiments, the method comprises reducing inflammatory biomarkers such as IL-6, IL-8, IL-10, IL1B, IL-12, IL-15, IL-17, CCL2, IL-1α, IL-2, IL-5, IL-9, CCL4, M-CSF, MCP-1, GCSF, MIP1A, CRP, TNFα, TNFβ, IFNγ, IP10, MCP1, and SAA1. In some embodiments, the method comprises reducing APACHE II score and/or KNAUS score. In some embodiments, the method comprises increasing point of NIAID 8-point ordinal scale. In some embodiments, the method comprises one or more of: i) reducing viral load in infected tissue and/or organ; or ii) reducing and/or preventing organ (e.g., lung) collagen deposition. In some embodiments, the method further comprises selecting the individual based on that the individual is at least about 55 years old (e.g., at least about any of 60, 65, 70, 75, 80, 85, 90 years old, or older).

In some embodiments, there is provided a method of reducing inflammation (e.g., viral activity, infiltration of inflammatory cells (e.g., CTL, NK cell, neutrophil, monocyte, macrophage), inflammatory biomarkers, cytokine storm, SIRS, sepsis, septic shock) due to H1N1 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of reducing cytokine storm due to H1N1 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of reducing inflammation (e.g., viral activity, infiltration of inflammatory cells (e.g., CTL, NK cell, neutrophil, monocyte, macrophage), inflammatory biomarkers, cytokine storm, SIRS, sepsis, septic shock) due to H1N1 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of reducing cytokine storm due to H1N1 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., oseltamivir, zanamivir, peramivir, lopinavir/ritonavir (Kaletra®, e.g., tablet), and/or IFN-α (e.g., via inhalation)). In some embodiments, the method comprises reducing inflammatory biomarkers such as IL-6, IL-8, IL-10, IL1B, IL-12, IL-15, IL-17, CCL2, IL-lca, IL-2, IL-5, IL-9, CCL4, M-CSF, MCP-1, GCSF, MIP1A, CRP, TNFα, TNFβ, IFNγ, IP10, MCP1, and SAA1. In some embodiments, the method comprises reducing APACHE II score and/or KNAUS score. In some embodiments, the method comprises increasing point of MAID 8-point ordinal scale. In some embodiments, the method comprises one or more of: i) reducing viral load in infected tissue and/or organ; or ii) reducing and/or preventing organ (e.g., lung) collagen deposition. In some embodiments, the method further comprises selecting the individual based on that the individual is at least about 55 years old (e.g., at least about any of 60, 65, 70, 75, 80, 85, 90 years old, or older).

In some embodiments, there is provided a method of reducing inflammation (e.g., viral activity, infiltration of inflammatory cells (e.g., CTL, NK cell, neutrophil, monocyte, macrophage), inflammatory biomarkers, cytokine storm, SIRS, sepsis, septic shock) due to H5N1 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of reducing cytokine storm due to H5N1 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of reducing inflammation (e.g., viral activity, infiltration of inflammatory cells (e.g., CTL, NK cell, neutrophil, monocyte, macrophage), inflammatory biomarkers, cytokine storm, SIRS, sepsis, septic shock) due to H5N1 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of reducing cytokine storm due to H5N1 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., oseltamivir, zanamivir, peramivir, lopinavir/ritonavir (Kaletra®, e.g., tablet), and/or IFN-α (e.g., via inhalation)). In some embodiments, the method comprises reducing inflammatory biomarkers such as IL-6, IL-8, IL-10, IL1B, IL-12, IL-15, IL-17, CCL2, IL-Ica, IL-2, IL-5, IL-9, CCL4, M-CSF, MCP-1, GCSF, MIP1A, CRP, TNFα, TNFβ, IFNγ, IP10, MCP1, and SAA1. In some embodiments, the method comprises reducing APACHE II score and/or KNAUS score. In some embodiments, the method comprises increasing point of MAID 8-point ordinal scale. In some embodiments, the method comprises one or more of: i) reducing viral load in infected tissue and/or organ; or ii) reducing and/or preventing organ (e.g., lung) collagen deposition. In some embodiments, the method further comprises selecting the individual based on that the individual is at least about 55 years old (e.g., at least about any of 60, 65, 70, 75, 80, 85, 90 years old, or older).

In some embodiments, there is provided a method of promoting regeneration of injured tissue or organ (e.g., lung, heart, kidney, liver) due to virus infection in an individual (e.g., human, such as a human of at least about 55 years old), comprising administering to the individual an effective amount of an IL-22 dimer. In some embodiments, there is provided a method of promoting regeneration of injured tissue or organ (e.g., lung, heart, kidney, liver) due to virus infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer and a dimerization domain. In some embodiments, the IL-22 monomer is connected to the dimerization domain via an optional linker. In some embodiments, the linker comprises the sequence of any one of SEQ ID NOs: 1-20 and 32. In some embodiments, the linker is about 6 to about 30 amino acids in length. In some embodiments, the linker comprises the sequence of SEQ ID NO: 1 or 10. In some embodiments, the dimerization domain comprises at least two cysteines capable of forming intermolecular disulfide bonds. In some embodiments, the dimerization domain comprises at least a portion of an Fc fragment. In some embodiments, the Fc fragment comprises CH2 and CH3 domains. In some embodiments, the Fc fragment comprises the sequence of SEQ ID NO: 22 or 23. In some embodiments, the IL-22 monomer comprises the sequence of SEQ ID NO: 21. In some embodiments, the IL-22 monomer is N-terminal to the dimerization domain. In some embodiments, the IL-22 monomer is C-terminal to the dimerization domain. In some embodiments, each monomeric subunit comprises the sequence of any of SEQ ID NOs: 24-27 (such as SEQ ID NO: 24). Thus in some embodiments, there is provided a method of promoting regeneration of injured tissue or organ (e.g., lung, heart, kidney, liver) due to virus infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the virus belongs to any one of the Orthomyxoviridae, Filoviridae, Flaviviridae, Coronaviridae, and Poxviridae families. In some embodiments, the virus is SARS-CoV, MERS-CoV, SARS-CoV-2, H1N1, or H5N1. In some embodiments, the method comprises upregulating regeneration biomarkers such as ANGPT2, FGF-b, PDGF-AA, Reg3A, and PDGF-BB. In some embodiments, the method comprises reducing ARDS score, APACHE II score, and/or KNAUS score. In some embodiments, the method comprises improving organ (e.g., lung, heart, liver, kidney) function test score. In some embodiments, the method comprises increasing point of NIAID 8-point ordinal scale. In some embodiments, the method comprises regenerating functional endothelial (e.g., pulmonary endothelial) cells and/or EGX. In some embodiments, the method further comprises selecting the individual based on that the individual is at least about 55 years old (e.g., at least about any of 60, 65, 70, 75, 80, 85, 90 years old, or older). In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., remdesivir, oseltamivir, zanamivir, peramivir, lopinavir/ritonavir (Kaletra®), and/or IFN-α).

Thus in some embodiments, there is provided a method of promoting regeneration of injured tissue or organ (e.g., lung, heart, kidney, liver) due to SARS-CoV infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of promoting regeneration of injured lung due to SARS-CoV infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of promoting regeneration of injured tissue or organ (e.g., lung, heart, kidney, liver) due to SARS-CoV infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of promoting regeneration of injured lung due to SARS-CoV infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., remdesivir, lopinavir/ritonavir (Kaletra®, e.g., tablet), and/or IFN-α (e.g., via inhalation)). In some embodiments, the method comprises upregulating regeneration biomarkers such as ANGPT2, FGF-b, PDGF-AA, Reg3A, and PDGF-BB. In some embodiments, the method comprises reducing ARDS score, APACHE II score, and/or KNAUS score. In some embodiments, the method comprises improving organ (e.g., lung, heart, liver, kidney) function test score. In some embodiments, the method comprises increasing point of NIAID 8-point ordinal scale. In some embodiments, the method comprises regenerating functional endothelial (e.g., pulmonary endothelial) cells and/or EGX. In some embodiments, the method further comprises selecting the individual based on that the individual is at least about 55 years old (e.g., at least about any of 60, 65, 70, 75, 80, 85, 90 years old, or older).

In some embodiments, there is provided a method of promoting regeneration of injured tissue or organ (e.g., lung, heart, kidney, liver) due to MERS-CoV infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of promoting regeneration of injured lung due to MERS-CoV infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of promoting regeneration of injured tissue or organ (e.g., lung, heart, kidney, liver) due to MERS-CoV infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of promoting regeneration of injured lung due to MERS-CoV infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., remdesivir, lopinavir/ritonavir (Kaletra®, e.g., tablet), and/or IFN-α (e.g., via inhalation)). In some embodiments, the method comprises upregulating regeneration biomarkers such as ANGPT2, FGF-b, PDGF-AA, Reg3A, and PDGF-BB. In some embodiments, the method comprises reducing ARDS score, APACHE II score, and/or KNAUS score. In some embodiments, the method comprises improving organ (e.g., lung, heart, liver, kidney) function test score. In some embodiments, the method comprises increasing point of NIAID 8-point ordinal scale. In some embodiments, the method comprises regenerating functional endothelial (e.g., pulmonary endothelial) cells and/or EGX. In some embodiments, the method further comprises selecting the individual based on that the individual is at least about 55 years old (e.g., at least about any of 60, 65, 70, 75, 80, 85, 90 years old, or older).

In some embodiments, there is provided a method of promoting regeneration of injured tissue or organ (e.g., lung, heart, kidney, liver) due to SARS-CoV-2 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of promoting regeneration of injured lung due to SARS-CoV-2 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of promoting regeneration of injured tissue or organ (e.g., lung, heart, kidney, liver) due to SARS-CoV-2 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of promoting regeneration of injured lung due to SARS-CoV-2 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., remdesivir, lopinavir/ritonavir (Kaletra®, e.g., tablet), and/or IFN-α (e.g., via inhalation)). In some embodiments, the method comprises upregulating regeneration biomarkers such as ANGPT2, FGF-b, PDGF-AA, Reg3A, and PDGF-BB. In some embodiments, the method comprises reducing ARDS score, APACHE II score, and/or KNAUS score. In some embodiments, the method comprises improving organ (e.g., lung, heart, liver, kidney) function test score. In some embodiments, the method comprises increasing point of MAID 8-point ordinal scale. In some embodiments, the method comprises regenerating functional endothelial (e.g., pulmonary endothelial) cells and/or EGX. In some embodiments, the method further comprises selecting the individual based on that the individual is at least about 55 years old (e.g., at least about any of 60, 65, 70, 75, 80, 85, 90 years old, or older).

In some embodiments, there is provided a method of promoting regeneration of injured tissue or organ (e.g., lung, heart, kidney, liver) due to H1N1 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of promoting regeneration of injured lung due to H1N1 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of promoting regeneration of injured tissue or organ (e.g., lung, heart, kidney, liver) due to H1N1 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of promoting regeneration of injured lung due to H1N1 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., oseltamivir, zanamivir, peramivir, lopinavir/ritonavir (Kaletra®, e.g., tablet), and/or IFN-α (e.g., via inhalation)). In some embodiments, the method comprises upregulating regeneration biomarkers such as ANGPT2, FGF-b, PDGF-AA, Reg3A, and PDGF-BB. In some embodiments, the method comprises reducing ARDS score, APACHE II score, and/or KNAUS score. In some embodiments, the method comprises improving organ (e.g., lung, heart, liver, kidney) function test score. In some embodiments, the method comprises increasing point of NIAID 8-point ordinal scale. In some embodiments, the method comprises regenerating functional endothelial (e.g., pulmonary endothelial) cells and/or EGX. In some embodiments, the method further comprises selecting the individual based on that the individual is at least about 55 years old (e.g., at least about any of 60, 65, 70, 75, 80, 85, 90 years old, or older).

In some embodiments, there is provided a method of promoting regeneration of injured tissue or organ (e.g., lung, heart, kidney, liver) due to H5N1 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of promoting regeneration of injured lung due to H5N1 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of promoting regeneration of injured tissue or organ (e.g., lung, heart, kidney, liver) due to H5N1 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of promoting regeneration of injured lung due to H5N1 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., oseltamivir, zanamivir, peramivir, lopinavir/ritonavir (Kaletra®, e.g., tablet), and/or IFN-α (e.g., via inhalation)). In some embodiments, the method comprises upregulating regeneration biomarkers such as ANGPT2, FGF-b, PDGF-AA, Reg3A, and PDGF-BB. In some embodiments, the method comprises reducing ARDS score, APACHE II score, and/or KNAUS score. In some embodiments, the method comprises improving organ (e.g., lung, heart, liver, kidney) function test score. In some embodiments, the method comprises increasing point of NIAID 8-point ordinal scale. In some embodiments, the method comprises regenerating functional endothelial (e.g., pulmonary endothelial) cells and/or EGX. In some embodiments, the method further comprises selecting the individual based on that the individual is at least about 55 years old (e.g., at least about any of 60, 65, 70, 75, 80, 85, 90 years old, or older).

In some embodiments, there is provided a method of treating or preventing endothelial (e.g., pulmonary endothelial) dysfunction (e.g., reducing EGX damage/shedding/degradation) in an injured tissue or organ (e.g., lung, heart, kidney, liver) due to virus (e.g., SARS-CoV, MERS-CoV, SARS-CoV-2, H1N1, H5N1) infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of treating or preventing endothelial (e.g., pulmonary endothelial) dysfunction (e.g., reducing EGX damage/shedding/degradation) in an injured tissue or organ (e.g., lung, heart, kidney, liver) due to virus (e.g., SARS-CoV, MERS-CoV, SARS-CoV-2, H1N1, H5N1) infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of treating or preventing endothelial (e.g., pulmonary endothelial) dysfunction (e.g., reducing EGX damage/shedding/degradation) in an injured tissue or organ (e.g., lung, heart, kidney, liver) due to SARS-CoV-2 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, there is provided a method of treating or preventing endothelial dysfunction (e.g., reducing EGX damage/shedding/degradation) in an injured lung due to SARS-CoV-2 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises an IL-22 monomer (e.g., SEQ ID NO: 21), a dimerization domain (e.g., Fc fragment, such as Fc fragment comprising SEQ ID NO: 22 or 23), and an optional linker (e.g., SEQ ID NO: 1 or 10) situated in between. In some embodiments, the IL-22 monomer is N-terminal to the dimerization domain. In some embodiments, the IL-22 monomer is C-terminal to the dimerization domain. Thus in some embodiments, there is provided a method of treating or preventing endothelial (e.g., pulmonary endothelial) dysfunction (e.g., reducing EGX damage/shedding/degradation) in an injured tissue or organ (e.g., lung, heart, kidney, liver) due to SARS-CoV-2 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, there is provided a method of treating or preventing endothelial dysfunction (e.g., reducing EGX damage/shedding/degradation) in an injured lung due to SARS-CoV-2 infection in an individual (e.g., human, such as a human of at least about 55 years old), comprising administering to the individual an effective amount of an IL-22 dimer, wherein the IL-22 dimer comprises two monomeric subunits, and wherein each monomeric subunit comprises the sequence of any of SEQ ID NOs: 24-27 (such as SEQ ID NO: 24). In some embodiments, the effective amount of the IL-22 dimer is about 2 μg/kg to about 200 μg/kg, about 5 μg/kg to about 80 μg/kg, about 10 μg/kg to about 45 μg/kg (e.g., 10 μg/kg, 30 μg/kg, or 45 μg/kg), or about 30 μg/kg to about 45 μg/kg. In some embodiments, the IL-22 dimer is administered intravenously, intrapulmonarily, or via inhalation or insufflation. In some embodiments, the IL-22 dimer is administered at least once a week. In some embodiments, the method further comprises administering to the individual an effective amount of another therapeutic agent, such as remdesivir, lopinavir/ritonavir (Kaletra®, e.g., tablet), IFN-α (e.g., via inhalation), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir, laninamivir, ribavirin (Rebetol®), umifenovir (Arbidol®), or any combinations thereof (e.g., remdesivir, lopinavir/ritonavir (Kaletra®, e.g., tablet), and/or IFN-α (e.g., via inhalation)). In some embodiments, EGX shedding is associated with increased fluid and protein leak and/or reduced integrity of the epithelium. In some embodiments, treating or preventing endothelial (e.g., pulmonary endothelial) dysfunction comprises one or more of the following: i) preventing and/or reducing EGX degradation, shedding, and/or damage; ii) down-regulating pro-inflammatory pathway such as TLR4 signaling; iii) promoting regeneration of functional endothelial cells and/or EGX; iv) protecting adherens junctions between endothelial cells and/or endothelial cell surface proteins, such as down-regulating extracellular proteinase (e.g., MMPs) expression, or up-regulating extracellular matrix protein expression (e.g., Tenascin C (Tnc), collagen, type I, alpha 1 (COL1a1), collagen, type VI, alpha 3 (Col6a3), and collagen, type I, alpha 2 (Col1a2)); v) preventing or reducing fluid and/or protein leakage; vi) reducing or preventing inflammatory cell (e.g., CTL, monocyte, neutrophil, macrophage, NK cell) infiltration; vii) restoring EGX-dependent barrier function; viii) recovering EGX-dependent cell-cell communication; ix) down-regulating inflammatory markers (e.g., IL-6, IL-8, IL-10, IL1B, IL-12, IL-15, IL-17, CCL2, IL-1α, IL-2, IL-5, IL-9, CCL4, M-CSF, MCP-1, GCSF, MIP1A, CRP, TNFα, TNFβ, IFNγ, IP10, MCP1, and SAA1); and (x) inducing endogenous IL-22 production. In some embodiments, the method further comprises selecting the individual based on that the individual is at least about 55 years old (e.g., at least about any of 60, 65, 70, 75, 80, 85, 90 years old, or older).

The individual to be treated can be any animals, such as a bird or a mammal. In some embodiments, the individual to be treated is a mammal, including, but is not limited to, livestock animals (e.g., cows, sheep, goats, donkeys, and horses), primates (e.g., human and non-human primates such as monkeys), feline, canine, rabbits, and rodents (e.g., mice, rats, gerbils, and hamsters). In some embodiments, the individual is a monkey (e.g., Cynomolgus monkey). In some embodiments, the individual is murine. In some embodiments, the individual is human.

In some embodiments, the individual (e.g., human) to be treated is about 5 years of age or younger, about 10 years of age or younger, about 16 years of age or younger, about 18 years of age or younger, about 20 years of age or younger, about 25 years of age or younger, about 35 years of age or younger, about 45 years of age or younger, about 55 years of age or younger, about 65 years of age or younger, about 75 years of age or younger, or about 85 years of age or younger. In some embodiments, the individual to be treated is about 5 years of age or older, about 10 years of age or older, about 16 years of age or older, about 18 years of age or older, about 20 years of age or older, about 25 years of age or older, about 35 years of age or older, about 45 years of age or older, about 55 years of age or older, about 60 years of age or older, about 65 years of age or older, about 70 years of age or older, about 75 years of age or older, about 80 years of age or older, about 85 years of age or older, or about 90 years of age or older. In some embodiments, the individual to be treated is between about 1 to about 90, about 5 to about 85, about 10 to about 80, about 15 to about 75, or about 18 to about 70 years of age.

In some embodiments, the individual administered with the IL-22 dimer does not show injection site reactions. In some embodiments, the individual administered with the IL-22 dimer does not show one or more adverse events such as dry skin, erythema, or nummular eczema, and/or significant abnormalities of the other safety evaluation indexes, such as physical examination, laboratory test, body weight, vital signs, electrocardiogram, and abdomen ultrasound, etc.

### Virus-Induced Organ Injury or Failure

Methods, compositions, combinations, and kits according to the present disclosure provide for the treatment of virus-induced organ injury or failure associated with the infection by a large number of viruses. The virus-induced tissue/organ injury or failure described herein can be associated with infection by any virus or combination of viruses, such as a virus of any one of the Orthomyxoviridae, Filoviridae, Flaviviridae, Coronaviridae, and Poxviridae families, or any combinations thereof, including identified and unidentified genera, species, subtypes, strains, and reassortants thereof.

The virus-induced injury or failure can occur to any tissue, organ, or system of the individual. In some embodiments, the virus-induced injury or failure is injury or failure at the respiratory system (e.g., pharynx, larynx, trachea, bronchi, lungs and diaphragm), circulatory system (e.g., lung, heart, blood vessel), muscular system (e.g., muscles), integumentary system (e.g., skin, hair, nail), digestive system (e.g., esophagus, stomach, liver, gallbladder, pancreas, intestines, colon, rectum), reproductive system (e.g., ovaries, fallopian tubes, uterus, vulva, vagina, testes, vas deferens, seminal vesicles, prostate, penis), endocrine system (e.g., hypothalamus, pituitary gland, pineal body or pineal gland, thyroid, parathyroids, adrenals), excretory system (e.g., kidneys, ureters, bladder, urethra), skeletal system (e.g., bones, cartilage, ligaments, tendons), lymphatic system (e.g., lymph node, tonsils, adenoids, thymus, spleen), or nervous system (e.g., brain, spinal cord, nerves). In some embodiments, the virus-induced injury or failure is injury or failure at the virus infected tissue or organ. For example, in some embodiments, a respiratory viral infection causes injury or failure to the respiratory track (e.g., lung). In some embodiments, the virus-induced injury or failure is injury or failure at a different site from the virus-infected tissue, organ, and/or system. For example, in some embodiments, a respiratory viral infection causes injury or failure to heart, kidney, liver, brain, or the gastrointestinal track. For example, SARS-CoV, MERS-CoV, and the newly identified SARS-CoV-2 not only causes injury and/or failure to the respiratory track (e.g., lung), leading to pneumonia (e.g., mild pneumonia, severe pneumonia, acute pneumonia), shortness of breath, breathing difficulty, pulmonary fibrosis, or ARDS, in many cases they also cause injury and/or failure to non-respiratory tissues/organs, such as heart, kidney, and liver, sepsis, septic shock, or MODS. In some embodiments, the virus-induced injury or failure is injury or failure at tissue/organ expressing IL-22 receptor, such as epithelial and stromal cells of liver, lung, skin, thymus, pancreas, kidney, gastrointestinal tract, synovial tissues, heart, breast, eye, and adipose tissue. In some embodiments, the virus-induced injury or failure is injury or failure at more than one tissue/organ. In some embodiments, the virus-induced injury or failure is injury or failure at tissue/organ comprising endothelial cells. In some embodiments, injured tissue or organ comprises endothelial cell injury, dysfunction, or death. In some embodiments, the endothelial cell is a pulmonary endothelial cell.

In some embodiments, the virus-induced injury or failure is heart injury or failure, such as myocardial infarction; congestive heart failure (CHF); myocardial failure; myocardial hypertrophy; ischemic cardiomyopathy; systolic heart failure; diastolic heart failure; stroke; thrombotic stroke; concentric LV hypertrophy, myocarditis; cardiomyopathy; hypertrophic cardiomyopathy; myocarditis; decompensated heart failure; ischemic myocardial disease; congenital heart disease; angina pectoris; prevention of heart remodeling or ventricular remodeling after myocardial infarction; ischemia-reperfusion injury in ischemic and post-ischemic events (e.g. myocardial infarct); mitral valve regurgitation; hypertension; hypotension; restenosis; fibrosis; thrombosis; platelet aggregation; or any cardiovascular diseases and their-complications associated with virus infection.

In some embodiments, the virus-induced injury or failure is a fibrotic condition. In some embodiments, said fibrotic conditions is selected from a group consisting of fibrotic conditions involving tissue remodeling following inflammation or ischemia-reperfusion injury, including but not limited to endomyocardial and cardiac fibrosis; mediastinal fibrosis; idiopathy pulmonary fibrosis; pulmonary fibrosis; retroperitoneal fibrosis; fibrosis of the spleen; fibrosis of the pancreas; hepatic fibrosis (cirrhosis) alcohol and non-alcohol related (including viral infection such as HAV, HBV and HCV); fibromatosis; granulomatous lung disease; glomerulonephritis myocardial scarring following infarction; endometrial fibrosis and endometriosis; wound healing. In some embodiments, the virus-induced injury or failure comprises increased collagen deposition.

In some embodiments, the virus-induced injury or failure is associated with endothelial dysfunction, injury, or death. In some embodiments, endothelial dysfunction comprises one or more of impairment of endothelium-dependent vasodilation, increased endothelial permeability, and endothelial glycocalyx (EGX) degradation, shedding, or damage. In some embodiments, the endothelial dysfunction comprises increased shedding or degradation of EGX. In some embodiments, EGX shedding is associated with increased fluid and protein leak and/or reduced integrity of the epithelium. In some embodiments, the virus-induced injury or failure is associated with endothelial dysfunction in a diseased tissue or organ of the subject. In some embodiments, the diseased tissue is lung.

In some embodiments, the virus-induced injury or failure is an endothelial dysfunction disease, such as cardiovascular diseases, high blood pressure, atherosclerosis, thrombosis, myocardial infarct, heart failure, renal diseases, plurimetabolic syndrome, erectile dysfunction; vasculitis; and diseases of the central nervous system (CNS).

In some embodiments, the virus-induced injury or failure is skin or tissue injury, such as lesions, wound healing.

In some embodiments, the virus-induced injury or failure is urogenital disorder or genitor-urological disorder, including but not limited to renal disease; a bladder disorder; disorders of the reproductive system; gynecologic disorders; urinary tract disorder; incontinence; disorders of the male (spermatogenesis, spermatic motility), and female reproductive system; sexual dysfunction; erectile dysfunction; embryogenesis; and conditions associated with pregnancy.

In some embodiments, the virus-induced injury or failure is a bone disease, such as Osteoporosis; Osteoarthritis; Osteopetrosis; Bone inconsistency; Osteosarcoma.

In some embodiments, the virus-induced injury or failure is ischemia-reperfusion injury associated with ischemic and post-ischemic events in organs and tissues in a patient, such as thrombotic stroke; myocardial infarction; angina pectoris; embolic vascular occlusions; peripheral vascular insufficiency; splanchnic artery occlusion; arterial occlusion by thrombi or embolisms, arterial occlusion by non-occlusive processes such as following low mesenteric flow or sepsis; mesenteric arterial occlusion; mesenteric vein occlusion; ischemia-reperfusion injury to the mesenteric microcirculation; ischemic acute renal failure; ischemia-reperfusion injury to the cerebral tissue; intestinal intussusception; hemodynamic shock; tissue dysfunction; organ failure; restenosis; atherosclerosis; thrombosis; platelet aggregation.

In some embodiments, the virus-induced injury or failure is an inflammatory condition associated with such infection, such as viral infection caused by human immunodeficiency virus I (HIV-1) or HIV-2, acquired immune deficiency (AIDS), West Nile encephalitis virus, coronavirus (e.g., SARS-CoV, MERS-CoV, SARS-CoV-2), rhinovirus, influenza virus (e.g., H1N1, H5N1), dengue virus, HCV, HBV, HAV, hemorrhagic fever; an otological infection; sepsis and sinusitis.

In some embodiments, the virus-induced injury or failure is an inflammatory disorder, such as gastritis, gout, gouty arthritis, arthritis, rheumatoid arthritis, inflammatory bowel disease, Crohn's disease, ulcerative colitis, ulcers, chronic bronchitis, asthma, allergy, acute lung injury, pulmonary inflammation, airway hyper-responsiveness, vasculitis, septic shock and inflammatory skin disorders, including but not limited to psoriasis, atopic dermatitis, eczema.

In some embodiments, the virus-induced organ injury or failure is kidney injury or failure, such as diabetic nephropathy; glomerulosclerosis; nephropathies; renal impairment; scleroderma renal crisis and chronic renal failure.

In some embodiments, the symptom of virus-induced tissue/organ injury or failure can be any viral infection symptoms, such as one or more of fever (temperature of >38° C.), cough, shortness of breath, breathing difficulty, pulmonary fibrosis, pneumonia, acute lung injury (ALI), acute respiratory distress syndrome (ARDS), multiple organ dysfunction syndrome (MODS), systemic inflammatory response syndrome (SIRS), cytokine storm, Zika fever (dengue-like fever) hypotension, tachycardia, dyspnea, ischemia, insufficient tissue perfusion (especially involving the major organs), uncontrollable hemorrhage, multisystem organ failure (caused primarily by hypoxia, tissue acidosis), severe metabolism dysregulation. In particular embodiments the symptom or damage associated with the viral infection is any one of fever, for example Zika fever, West Nile fever, Dengue fever or Yellow fever, where fever is usually accompanied by at least one of headaches, vomiting, skin rash, muscle and joint pains, and a characteristic skin rash, and other effects, e.g. as described above. In some embodiments, the methods described herein can control, ameliorate, and/or prevent one or more of symptoms associated with virus-induced organ injury or failure. Treatment in accordance with the present disclosure in some embodiments can prevent death of the treated subject.

In some embodiments, the expression levels of gene products (e.g., biomarkers) in a biological sample (e.g., sputum/saliva, blood, urine, feces, cerebrospinal fluid, or body disuse) are particularly indicative of the presence and/or severity of virus infection, inflammation, cytokine storm, organ injury, organ failure, SIRS, sepsis, septic shock, or MODS. In some embodiments, the expression levels of gene products (e.g., biomarkers) in a biological sample (e.g., sputum/saliva, blood, urine, feces, cerebrospinal fluid, or body disuse) are indicative of therapeutic effect of the methods described herein, e.g., the decrease of inflammatory cytokines and/or the increase of regeneration markers are indicative of effective treatment. Said blood sample preferably comprises whole blood, platelets, peripheral blood mononuclear cells (PBMCs), and/or buffy coat. In some embodiments, said sample is a whole blood sample. An expression product of a gene comprises for instance nucleic acid molecules and/or proteins. In some embodiments, the gene product shows virus genetic information, such as virus DNA, virus RNA, or viral protein (e.g., envelope protein). Preferably, said product is isolated from said sample of said individual.

Analysis of expression products according to the invention can be performed with any method known in the art. Protein levels are for instance measured using antibody-based binding assays. Enzyme labeled, radioactively labeled or fluorescently labeled antibodies are for instance used for detection and quantification of protein. Assays that are for instance suitable include enzyme-linked immunosorbent assays (ELISA), radio-immuno assays (RIA), Western Blot assays and immunohistochemical staining assays. Alternatively, in order to determine the expression level of multiple proteins simultaneously protein arrays such as antibody-arrays are for instance used.

In some embodiments, the presence or level of DNA (e.g., virus DNA) is tested. Any laboratory techniques for DNA detection and/or measurement can be used, such as PCR, qPCR, DNA-seq, DNA array, or DNA probe.

In some embodiments, an expression product comprises RNA, such as total RNA or mRNA. In some embodiments, the presence or level of RNA (e.g., virus RNA) is tested. The lifespan of RNA molecules is shorter than the lifespan of proteins. RNA levels are therefore more representative of the status of an individual at the time of sample preparation, and thus are more suitable for determining the presence and/or severity of inflammation, cytokine storm, organ injury, organ failure, SIRS, sepsis, septic shock, or MODS in an individual suffering from a virus infection. Furthermore, determining RNA expression levels is less laborious than determining protein levels. For instance, oligonucleotides arrays are used that are easier to develop and process than protein chips. In some embodiments, RT-PCT, qRT-PCR, RNA-seq, RNA probe, or Northern blot is used to detect and/or measure said RNA product.

Virus-induced organ injury or failure, or the therapeutic effect of the methods described herein, can also be determined by established function tests for said organ, medical imaging (e.g., CT imaging, MRI) of organ site, biopsy of such organ, or histopathology study. The improvement of function test scores or pathology of said organ to normal ranges can be indicative of effective treatment.

Also see Examples herein for possible indicators and measurements.

Lung Injury or Failure

In some embodiments, the virus-induced injury or failure is respiratory system injury or failure, such as lung injury or failure, e.g., asthma, acute lung injury (ALI), bronchial disease, lung diseases, pneumonia (e.g., mild pneumonia, severe pneumonia), acute pneumonia, chronic obstructive pulmonary disease (COPD), Acute Respiratory Distress Syndrome (ARDS), SARS, MERS, Coronavirus disease 2019 (COVID-19), fibrosis related asthma, cystic fibrosis, pulmonary fibrosis. In some embodiments, the virus-induced organ injury or failure is SARS. In some embodiments, the virus-induced organ injury or failure is MERS. In some embodiments, the virus-induced organ injury or failure is COVID-19. In some embodiments, the virus-induced organ injury or failure is H1N1 swine flu. In some embodiments, the virus-induced organ injury or failure is H5N1 bird flu. In some embodiments, the virus-induced respiratory system injury or failure is characterized by endothelial dysfunction/injury/death, and/or EGX shedding/damage. Any suitable methods can be used to measure EGX, e.g., staining with WGA and 4′,6-diamidino-2-phenylindole then imaging using a microscopy method. Also see Examples 3 and 4 for exemplary methods.

In some embodiments, the methods described herein may be used to treat or prevent the inflammatory effects of viral infection of the upper or lower respiratory tracts. In particular, the methods described herein may be used to treat or prevent respiratory failure caused by viral infection, including acute lung injury or acute respiratory distress syndrome. In some embodiments, the methods described herein may also be used to treat or prevent the sequelae of respiratory failure caused by viral infection, including multi-organ failure or MODS.

In some embodiments, the virus-induced lung injury or failure is pulmonary fibrosis, pneumonia, ALI, or acute respiratory distress syndrome (ARDS). ARDS, the most severe form of acute lung injury (ALI), is a devastating clinical syndrome with high mortality rate (30-60%). ARDS is a type of respiratory failure characterized by rapid onset of widespread inflammation in the lungs. Symptoms may include shortness of breath, fast breathing, and a low oxygen level in the blood due to abnormal ventilation. Other common symptoms include muscle fatigue and general weakness, low blood pressure, a dry, hacking cough, and fever.

Degradation of the glycocalyx has been implicated in the fluid and protein leak that occurs in ARDS, and protection of the glycocalyx after lung injury mitigates the changes seen in the lung during ARDS (Murphy, L. S., et al., “Endothelial glycocalyx degradation is more severe in patients with non-pulmonary sepsis compared to pulmonary sepsis and associates with risk of ARDS and other organ dysfunction.” Annals of Intensive Care, 2017. 7(1): p. 1-9; Kong, G., et al., “Astilbin alleviates LPS-induced ARDS by suppressing MAPK signaling pathway and protecting pulmonary endothelial glycocalyx.” Int Immunopharmacol, 2016. 36: p. 51-58; Wang, L., et al., “Ulinastatin attenuates pulmonary endothelial glycocalyx damage and inhibits endothelial heparanase activity in LPS-induced ARDS.” Biochem Biophys Res Commun, 2016. 478(2): p. 669-75).

Pulmonary function tests (PFTs) can be used to determine the presence and/or severity of lung injury or failure, or to determine therapeutic efficacy of a treatment. PFTs are noninvasive tests that show how well the lungs are working. The tests measure lung volume, capacity, rates of flow, and gas exchange. Spirometry is used to screen for diseases that affect lung volumes, or the airways, such as COPD or asthma. Lung volume testing is another test that is more precise than spirometry and measures the volume of air in the lungs, including the air that remains at the end of a normal breath. A diffusing capacity test measures how easily oxygen enters the bloodstream. In some embodiments, the treatment effect can be determined by PFTs measuring one or more of: tidal volume (VT), minute volume (MV), vital capacity (VC), functional residual capacity (FRC), residual volume, total lung capacity, forced vital capacity (FVC), forced expiratory volume (FEV), forced expiratory flow (FEF), and peak expiratory flow rate (PEFR). The improvement of one or more of such PFT indicators from malfunction ranges to standard/healthy ranges can be indicative of the treatment effect of the methods described herein.

Lung functional studies can be conducted under tidal breathing conditions (Goplen et al. J Allergy Clin Immunol. 2009; 123(4): 925-32.e11). Various perturbations can be performed before and following deep inflation which recruits closed airways. These measurements can be compared to pre-inflation data to determine baseline vs. lung capacity lung physiology for single compartment, constant phase, and pressure volume loops on a flexiVent® (Scireq) computer controlled piston respirator. Several parameters can be measured to reflect lung functions, such as input impedance (Zrs), resistance (R), compliance (C), tissue damping (G), etc. Also see Example 7 for example. In some embodiments, the methods described herein (e.g., preventing or treating a virus-induced lung injury or failure, or protecting lung from virus-induced lung injury or failure) improve lung function, which can comprise one or more of the following: i) improving baseline function of lung parenchyma; ii) decreasing resistance to airflow, e.g., in small airways; iii) improving alveolar use; iv) preventing airway collapse; and v) increasing compliance (decreasing lung stiffness).

The effects of IL-22 dimer on preventing or treating a virus-induced lung injury or failure, or protecting lung from virus-induced lung injury or failure can be measured using the NIAID 8-point ordinal scale: 1. Death; 2. Hospitalized, on invasive mechanical ventilation or extracorporeal membrane oxygenation; 3. Hospitalized, on non-invasive ventilation or high-flow oxygen devices; 4. Hospitalized, requiring supplemental oxygen; 5. Hospitalized, not requiring supplementation oxygen—requiring ongoing medical care (COVID-19 related or otherwise); 6. Hospitalized, not requiring supplemental oxygen—no longer requires ongoing medical care; 7. Not hospitalized, limitation on activities and/or requiring home oxygen; and 8. Not hospitalized, no limitations on activities. In some embodiments, the methods described herein increase at least 1-point (e.g., at least 2, 3, 4, 5, or more points) in the NIAID scale. Also see Example 5.

Virus-induced lung injury or failure, or the therapeutic effect of methods described herein, can also be determined by medical imaging (e.g., CT imaging, MRI) of the chest, lung biopsy, and pulmonary histopathology scores (see Examples 1, 4, and 7 for possible measurements). Histology studies can be conducted with any known methods. Paraffin-embedded lungs from virus-infected individual can be sliced and stained with dyes such as hematoxylin and eosin (H&E), Masson's Trichrome, Sirius Red, Periodic acid-Schiff (PAS), etc. For example, CT imaging from patients of SARS-CoV-2 infection often shows bilateral pulmonary parenchymal ground-glass and consolidative pulmonary opacities, sometimes with a rounded morphology and a peripheral lung distribution. Mild or moderate progression of disease is manifested by increasing extent and density of lung opacities.

Viral load in virus-infected tissue or organ can be examined by extracting total RNAs from cell lysates, the subjecting to subgenomic-N (sgm-N) RNA standard assay (subgenomic RNA measures new viral RNA, not just the viral inoculum), or RNA-seq (e.g., determining the read counts per virus ORF). Also see Example 6 for example.

Reduction of the inflammatory effects of viral infection of the respiratory tract may also be assessed by reduction in inflammatory cytokines (e.g., CXCL2, IL-1β, and/or IL-6) and/or inflammatory cells (e.g., CTL, NK cell, neutrophil, monocyte, macrophage) in a subject suffering from such a viral infection. Cytokine levels and inflammatory cell levels may, for example, be assessed in bronchoalveolar lavage (BAL) fluid from the subject. Inflammatory cell infiltration can also be examined by immunofluorescence staining, then lung tissue can be harvested, digested, and subjected to FACS sorting. Also see Example 7 for example.

Multiple Organ Dysfunction Syndrome (MODS)

Multiple organ dysfunction syndrome (MODS), also known as multiple organ failure (MOF), total organ failure (TOF), or multisystem organ failure (MSOF), is altered organ function in an acutely ill patient such that homeostasis cannot be maintained without medical intervention. MODS is generally defined as the presence of failure in at least two organ systems. MODS usually results from uncontrolled inflammatory response triggered by infection, injury (accident, surgery), hypoperfusion, and hypermetabolism. The uncontrolled inflammatory response can lead to sepsis or Systemic Inflammatory Response Syndrome (SIRS). SIRS is an inflammatory state affecting the whole body. It is one of several conditions related to systemic inflammation, organ dysfunction, and organ failure. SIRS is a subset of cytokine storm, in which there is abnormal regulation of various cytokines. The cause of SIRS can be infectious or noninfectious. SIRS is closely related to sepsis. When SIRS is due to an infection, it is considered as sepsis. Noninfectious causes of SIRS include trauma, burns, pancreatitis, ischemia, and hemorrhage. Sepsis is a serious medical condition characterized by a whole-body inflammatory state, and can lead to septic shock. Both SIRS and sepsis can progress to severe sepsis, and eventually MODS, or death. The underline mechanism of MODS is not well understood. The chance of survival generally reduces with an increasing number of organs involved in MODS. Examples of failure organ systems are failure of the respiratory system (e.g., lung), failure of hepatic, renal or gastrointestinal function and circulatory failure.

Treatment of MODS is non-specific and mainly supportive including for instance treatment of infection, nutritional support and artificial support for individual failed organs, such as dialysis and tissue perfusion or oxygenation. Several immunomodulatory interventions, including treatment with immunoglobulin or IFNγ, have been tested, with a low rate of success.

The development of MODS in a patient is currently established by classification systems such as the KNAUS criteria for Multiple System Organ Failure (Knaus, W A et al. Ann. Surg. 1985; 202:685-293), which involve physiological measurement such as respiratory frequency, heart rate and arterial pressure, urine volume, serum creatinine, and a patient questionnaire, resulting in a score on a scale of 1 to 10. A KNAUS score of 5 or higher is indicative of the presence of MODS. The KNAUS score is determined daily for patients at risk of developing MODS. Currently no methods are available which enable assessment of the risk of developing MODS, before the first signs of MODS become apparent. In some embodiments, the methods described herein can reduce KNAUS score, indicative of effective treatment.

Orthomyxoviridae

Orthomyxoviridae is a family of RNA viruses. It includes seven genera: Influenzavirus A, Influenzavirus B, Influenzavirus C, Influenzavirus D, Isavirus, Thogotovirus, and Quaranjavirus. The first four genera contain viruses that cause influenza in vertebrates, including birds (i.e., avian influenza), humans, and other mammals. Isaviruses infect salmon. Thogotoviruses are arboviruses and infect vertebrates and invertebrates, such as ticks and mosquitoes. Of the four genera of Influenza virus, Influenzavirus A infects humans, other mammals, and birds, and causes all flu pandemics; Influenzavirus B infects humans and seals; Influenzavirus C infects humans, pigs, and dogs; and Influenzavirus D infects pigs and cattle.

Influenza A and B virus particles contain a genome of negative sense, single-strand RNA divided into 8 linear segments. Co-infection of a single host with two different influenza viruses may result in the generation of reassortant progeny viruses having a new combination of genome segments, derived from each of the parental viruses.

Influenza A viruses are the most infectious human pathogens among the three influenza types and can cause the most severe diseases. They are further classified based on the viral surface proteins hemagglutinin (HA or H) and neuraminidase (NA or N). Sixteen H subtypes (or serotypes) and nine N subtypes of influenza A virus have been identified. Subtypes of influenza A virus are named according to their HA and NA surface proteins. For example, an “H7N2 virus” designates influenza A subtype that has an HA 7 protein and an NA 2 protein, etc. The serotypes that have been confirmed in humans include Influenza A virus subtype H1N1 (H1N1) which caused the “swine flu” in 2009; H2N2 caused “Asian Flu”; H3N2 caused “Hong Kong Flu”; Influenza A virus subtype H5N1 (H5N1) is a pandemic threat and causes avian influenza or “bird flu”; H7N7 has unusual zoonotic potential; H1N2 is endemic in humans and pigs; H9N2; H7N2; H7N3; and H10N7.

The 2009 flu pandemic (swine flu) caused by H1N1 was initially seen in the United States. The symptoms in human are generally similar to those of influenza and of influenza-like illness, including fever; cough, sore throat, watery eyes, body aches, shortness of breath, headache, weight loss, chills, sneezing, runny nose, coughing, dizziness, abdominal pain, lack of appetite and fatigue. Diarrhea and vomiting are seen in patients as well. Among the numerous causes of death such as pneumonia (leading to sepsis), high fever (leading to neurological problems), dehydration (from excessive vomiting and diarrhea), electrolyte imbalance, and kidney failure, respiratory failure is the most common cause of death. Young children and the elderly are affected the most. The primary lung pathology of fatal H1N1 influenza is characterized by necrotizing alveolitis and dense neutrophil infiltration.

All known subtypes of A viruses can be found in birds. Avian influenza or “bird flu” caused by H5N1 has killed millions of poultry. It is shown that person-to person transmission can also be adapted. The mortality due to respiratory and multi-organ failure is around 60%. Symptoms of human infection with avian viruses have ranged from typical flu-like symptoms (fever, cough, sore throat and muscle aches) to eye infections, pneumonia, severe respiratory diseases (such as acute respiratory distress), and other severe and life-threatening complications. The symptoms of bird flu may depend on which virus caused the infection. Each of avian influenza A viruses H5, H7, and H9 theoretically can be partnered with any one of nine neuraminidase surface proteins; thus, there are potentially nine different forms of each subtype (e.g., H5N1 to H5N9). H5 infections have been documented in humans, sometimes causing severe illness and death. H7 infection in humans is rare, but can occur among persons who have direct contact with infected birds. It is believed that most cases of bird flu infection in humans have resulted from contact with infected poultry or contaminated surfaces. The risk from bird flu is generally low to most people because the viruses occur mainly among birds and do not usually infect humans. However, the outbreak of avian influenza A (H5N1) among poultry in Asia and Europe is an example of a bird flu outbreak that has caused human infections and deaths. In some embodiments, the viral pathogen is avian Influenza virus type A virus, or any subtype and reassortant thereof. In some embodiments, the avian Influenza type A virus has haemagglutinin component of subtype H5, H7 or H9.

Reassortment and new Influenza subtype formation Influenza A viruses are found in many different animals, including ducks, chickens, pigs, whales, horses, and seals. However, certain subtypes of influenza A virus are specific to certain species, except for birds which are hosts to all subtypes of influenza A. Influenza A viruses normally seen in one species can cross over and cause illness in another species. For example, H5N1 avian influenza was responsible for an outbreak of bird flu in the human population, while H7N7, H9N2 and H7N2 subtypes have also been associated with transmission over the species barrier and resultant infection in humans. Avian influenza viruses may be transmitted to humans in two main ways; (a) directly from infected birds or from material contaminated with avian influenza virus, (b) through an intermediate host, such as a pig.

In some embodiments, the virus described herein is an Orthomyxoviridae virus selected from the group consisting of Influenza A virus, Influenza B virus, Influenza C virus, and any subtype or reassortant thereof. In some embodiments, the virus is an Influenza A virus or any subtype or reassortant thereof. In some embodiments, the virus is Influenza A virus subtype H1N1 (H1N1) or Influenza A virus subtype H5N1 (H5N1). In some embodiments, the virus-induced organ injury or failure is H1N1 swine flu. In some embodiments, the virus-induced organ injury or failure is H5N1 bird flu.

Filoviridae

In some embodiments, the viral pathogen can be a virus belonging to the Filoviridea family, also referred to herein as “Filoviruses.” These are generally single-stranded negative sense RNA viruses that typically infect primates. Filoviruses are able to multiply in virtually all cell types. The filovirus genome comprises seven genes that encode 4 virion structural proteins (VP30, VP35, nucleoprotein, and a polymerase protein (L-pol)) and 3 membrane-associated proteins (VP40, glycoprotein (GP), and VP24). Filoviruses cause hemorrhagic fevers with high levels of fatality. They are classified in two genera within the family Filoviridae: Ebola virus (EBOV) and Marburg virus (MARV), both being highly pathogenic in humans and nonhuman primates, with case fatality levels of up to 90%. Ebola virus species Reston (REBOV) is pathogenic in monkeys but does not cause disease in humans or great apes. Fatal outcome in filoviral infection is associated with an early reduction in the number of circulating T cells, failure to develop specific humoral immunity, and the release of pro-inflammatory cytokines. More specifically, these viruses cause sporadic epidemics of human disease characterized by systemic hemorrhage, multi-organ failure and death in most instances. The onset of illness is abrupt, and initial symptoms resemble those of an influenza-like syndrome. Fever, headache, general malaise, myalgia, joint pain, and sore throat are commonly followed by diarrhea and abdominal pain. A transient morbilliform skin rash, which subsequently desquamates, often appears at the end of the first week of illness. Other physical findings include pharyngitis, which is frequently exudative, and occasionally conjunctivitis, jaundice, and edema. After the third day of illness, hemorrhagic manifestations are common and include petechiae as well as frank bleeding, which can arise from any part of the gastrointestinal tract and from multiple other sites. As the disease progresses, patients develop severe multifocal necroses and a syndrome resembling septic shock. In addition, activation of the fibrinolytic system coupled with the consumption of coagulation factors results in a depletion of clotting factors and degradation of platelet membrane glycoproteins.

In some embodiments, the virus described herein is a Filoviridae virus selected from Ebola virus (EBOV) and Marburg virus (MARV).

Flaviviridae

In some embodiments, the viral pathogen can be a virus belonging to the Flaviviridea family, also referred to herein as “Flaviviruses,” a group of ssRNA(+) viruses. Humans and other mammals serve as natural hosts. The Flaviviridea family has four genera, including Genus Flavivirus which are usually mosquito-borne (type species Yellow fever virus (YFV), others include West Nile virus (WNV), Dengue virus (DENV), and Zika virus (ZIKV)), Genus Hepacivirus (type species Hepacivirus C (hepatitis C virus), also includes Hepacivirus B (GB virus B)), Genus Pegivirus (includes Pegivirus A (GB virus A), Pegivirus C (GB virus C), and Pegivirus B (GB virus D)), and Genus Pestivirus which infect non-human mammals (type species Pestivirus A (bovine viral diarrhea virus 1), others include Pestivirus C (classical swine fever virus, previously hog cholera virus)). This family also has a number of unclassified species.

WNV causes West Nile Fever, which can be manifested by fever, headache, vomiting, or a rash. Encephalitis or meningitis are rather rare. Recovery may take weeks to months.

DENV is the cause for Dengue fever (DF), with symptoms typically beginning three to fourteen days after infection, which may include a high fever, headache, vomiting, muscle and joint pains, and a characteristic skin rash. Recovery generally takes two to seven days. In a small proportion of cases, the disease develops into the life-threatening dengue hemorrhagic fever, resulting in bleeding, low levels of blood platelets and blood plasma leakage, or into dengue shock syndrome, where dangerously low blood pressure occurs.

YFV causes Yellow Fever, viral disease of typically short duration. In most cases, symptoms include fever, chills, loss of appetite, nausea, muscle pains particularly in the back, and headaches. Symptoms typically improve within five days. In about 15% of people, within a day of improving the fever comes back, abdominal pain occurs, and liver damage begins causing yellow skin. If this occurs, the risk of bleeding and kidney problems is also increased.

ZIKV causes a self-limiting, dengue fever (DF)-like disease with an incubation time of up to 10 days. Signs and symptoms consist of rather low-grade fever, myalgia and a maculopapular rash, accompanied by arthralgia and headache, and less often edema, sore throat, and vomiting. There have been ZIKV outbreaks in 2007 and in 2013, and an epidemic after its introduction to Brazil in 2016, all attributed to the Asian genotype of ZIKV. In contrast to DF, acute Zika fever (ZF) is less severe. A study has shown that polyfunctional T cell activation (Th1, Th2, Th9 and Th17 response) was seen during the acute phase of Zika fever, characterized by increase in respective cytokines levels (IL-2, IL-3, IL-13, IL-9 and IL-17), followed by a decrease in the reconvalescent phase. ZIKV infections are associated with Gillain-Barre syndrome (Tappe et al., Med Microbiol Immunol. 2016; 205:269-273). In pregnancy, the disease spreads from mother to fetus in the womb, and can cause multiple problems in the baby, most notably microcephaly, as well as eye abnormalities and hydrops fetalis.

In some embodiments, the virus described herein is a Flaviviridae virus selected from the group consisting of Zika virus (ZIKV), West Nile virus (WNV), Dengue virus (DENV), and Yellow Fever virus (YFV).

Coronaviridae

In some embodiments, the viral pathogen is a Coronaviridae family member. Coronaviridae viruses are enveloped, positive-sense, single-stranded RNA viruses. The particles often have large, club- or petal-shaped surface projections (“peplomers” or “spikes”), creating an image similar to solar corona in electron micrographs of spherical particles. The family Coronaviridae is organized in 2 sub-families, 5 genera, 23 sub-genera and about 40 species.

In some embodiments, the virus described herein is a Coronaviridae virus selected from the group consisting of alpha coronaviruses 229E (HCoV-229E), New Haven coronavirus NL63 (HCoV-NL63), beta coronaviruses OC43 (HCoV-OC43), coronavirus HKU1 (HCoV-HKU1), Severe Acute Respiratory Syndrome coronavirus (SARS-CoV), Middle East Respiratory Syndrome coronavirus (MERS-CoV), and Severe Acute Respiratory Syndrome Coronavirus 2 (SARS-CoV-2). In some embodiments, the virus-induced organ injury or failure is associated with SARS-CoV infection. In some embodiments, the virus-induced organ injury or failure is SARS. In some embodiments, the virus-induced organ injury or failure is associated with MERS-CoV infection. In some embodiments, the virus-induced organ injury or failure is MERS. In some embodiments, the virus-induced organ injury or failure is associated with SARS-CoV-2 infection. In some embodiments, the virus-induced organ injury or failure is COVID-19.

In some embodiments, the Coronaviridae virus is Severe Acute Respiratory Syndrome (SARS) coronavirus (SARS-CoV), causing a viral respiratory disease of zoonotic origin (outbreaks in 2002-2003, in southern China caused an eventual 8,098 cases, resulting in 774 deaths reported in 37 countries). Initial symptoms are flu-like and may include fever, muscle pain, lethargy symptoms, cough, sore throat, and other nonspecific symptoms. The only symptom common to all patients appears to be a fever above 38° C. (100° F.). SARS may eventually lead to shortness of breath and/or pneumonia—either direct viral pneumonia or secondary bacterial pneumonia. The average incubation period for SARS is 4-6 days, although rarely it could be as short as 1 day or as long as 14 days. There have been no outbreaks since 2004. No vaccine is available. The mortality associated with SARS is linked to rapidly progressive respiratory failure causing acute lung injury (ALI) or acute respiratory distress syndrome (ARDS). In some cases, multi-organ failure is also a feature. It was initially assumed that respiratory failure associated with SARS was due to rapid viral replication leading to cytolytic destruction of target cells of the respiratory tract, such as alveolar epithelial cells, or due to escape of the virus to tissues and organs remote from the respiratory system, such as the central nervous system. More evidence has shown, however, that the development of respiratory failure is not associated with high viral titres. Investigators have instead found that respiratory failure is associated with significant elevation of pro-inflammatory cytokines such as TFNα and IFNβ, leading to the inappropriate stimulation of the innate immune system triggering a so-called “cytokine storm.” A correlation between cytokine storm and severity of illness was found in SARS patients.

In some embodiments, the Coronaviridae virus is Middle East Respiratory Syndrome coronavirus (MERS-CoV). MERS-CoV is a betacoronavirus reported in 2012 in Saudi Arabia, and was identified as “threat to global health” by WHO. It is a highly pathogenic respiratory virus that causes severe respiratory distress and potentially renal failure in infected individuals. About 3 or 4 out of every 10 patients reported with MERS have died. Symptoms include fever, cough, diarrhea and shortness of breath. For many people with MERS, more severe complications followed, such as pneumonia (severe pneumonia can lead to ARDS), septic shock, and organ (e.g., kidney) failure. Disseminated intravascular coagulation (DIC), and pericarditis have also been reported. Similar to SARS, a correlation between cytokine storm and severity of illness was found in MERS patients.

The newest addition of the Coronaviridae family is the 2019 novel coronavirus (2019-nCoV), showing so far a lower mortality rate than the MERS- and SARS-coronavirus members. WHO has officially designated 2019-nCoV as “Severe Acute Respiratory Syndrome Coronavirus 2” (SARS-CoV-2). SARS-CoV-2 causes the 2019-2021 outbreak of an acute respiratory disease (“Coronavirus disease 2019”, COVID-19), designated as a global health emergency by the WHO. The genetic sequences of SARS-CoV-2 is similar to SARS-CoV (79.5%) and bat coronaviruses (96%). The viruses are primarily spread through close contact, in particular through respiratory droplets from coughs and sneezes. The average incubation period for SARS-CoV-2 is about 14 days. For confirmed SARS-CoV-2 infections, reported illnesses have ranged from people with little to no symptoms to people being severely ill and dying. Symptoms include fever, cough, sore throat, nasal congestion, malaise, headache, muscle pain, malaise, shortness of breath, pulmonary fibrosis, mild pneumonia, severe pneumonia, acute pneumonia, ALI, ARDS, sepsis (organ dysfunction), or septic shock. Signs of organ dysfunction include: altered mental status, difficult or fast breathing, low oxygen saturation, reduced urine output, fast heart rate, weak pulse, cold extremities or low blood pressure, skin mottling, or laboratory evidence of coagulopathy, thrombocytopenia, acidosis, high lactate or hyperbilirubinemia. Older individuals have significantly worse outcomes. A few vaccines just became available but limited. Scientists noticed that SARS-CoV-2 patients that were admitted to the ICU, particularly those with severe disease, showed significantly higher levels of inflammatory cytokines compared to those who did not. Such correlation between cytokine storm and severity of illness was previously observed in SARS and MERS patients. This “cytokine storm” can trigger excessive, uncontrolled systemic inflammation, leading to pneumonitis, ARDS, respiratory failure, shock, organ failure, secondary bacterial pneumonia, and potentially death.

Poxviridae

In some embodiments, the viral pathogen can be a virus belonging to the Poxviridae family. Poxviridae viruses have double-stranded DNA genome, and are generally enveloped. Humans, vertebrates, and arthropods serve as natural hosts. Diseases associated with this family include smallpox. Currently there are 69 species, divided among 28 genera, which are divided into two subfamilies. The four genera that are infectious to humans are: orthopoxvirus, parapoxvirus, yatapoxvirus, and molluscipoxvirus. Orthopox viruses include smallpox virus (variola), vaccinia virus, cowpox virus, and monkeypox virus. Parapox viruses include orf virus, pseudocowpox, and bovine papular stomatitis virus. Yatapox viruses include tanapox virus and yaba monkey tumor virus. Molluscipox viruses include molluscum contagiosum virus (MCV). The prototypical poxvirus is vaccinia virus, known for its role in the eradication of smallpox.

Smallpox was an infectious disease. WHO certified the global eradication of the disease in 1980. The risk of death was about 30%, with higher rates among babies. The malignant and hemorrhagic forms were usually fatal. Those who survived often had extensive scarring of their skin, and some were left blind. Symptoms of smallpox included fever, vomiting, muscle pain, nausea, formation of sores in the mouth and a skin rash.

### IL-22 Dimer

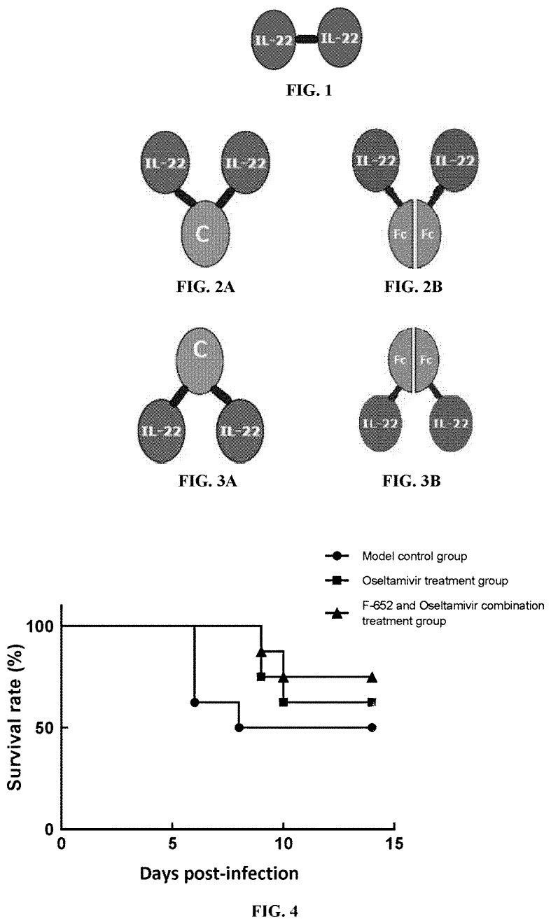

As used herein, the term “IL-22 dimer” refers to a protein comprising two units of an IL-22 protein, or two units of any of the IL-22 monomers described herein. For one example, an IL-22 dimer may comprise two IL-22 monomers directly connected to each other, or connected together via a linking moiety such as a peptide linker, a chemical bond, a covalent bond, or a polypeptide (e.g., carrier protein, dimerization domain). In some embodiments, the IL-22 dimer comprises two identical IL-22 monomers. In other embodiments, the IL-22 dimer comprises two different IL-22 monomers. Further examples of IL-22 dimers that may find use in the present inventions are described in U.S. Pat. No. 8,945,528, incorporated herein by reference in its entirety. In some embodiments, the IL-22 dimer is a recombinant IL-22 dimerized protein comprising two human IL-22 molecules and produced in transformed Chinese Hamster Ovary (CHO) cells in serum-free culture produced by Generon (Shanghai) Corporation Ltd. (now Evive Biotechnology (Shanghai) Ltd). IL-22 dimers are described, for example, in U.S. Pat. No. 8,945,528, including sequence information, incorporated herein by reference in its entirety. IL-22 dimer forming polypeptides used herein may be isolated from a variety of sources, such as from human tissue types or from another source, or prepared by recombinant or synthetic methods. In some embodiments, the IL-22 dimer comprises a carrier protein, including but not limited to an Fc fragment of an immunoglobulin (e.g., human IgG1, IgG2, IgG3, IgG4), or albumin (e.g., human albumin). The IL-22 monomer can be localized at the C-terminal or N-terminal of the carrier protein. In some embodiments, the IL-22 dimer does not comprise a carrier protein. FIGS. 1-3B illustrate representative structures of the IL-22 dimer of the present invention.

In some embodiments, the IL-22 dimer comprises Formula I: M1-L-M2; wherein Ml is a first IL-22 monomer, M2 is a second IL-22 monomer, and L is a linking moiety connecting the first IL-22 monomer and the second IL-22 monomer and disposed therebetween. In some embodiments, the first IL-22 monomer and the second IL-22 monomer are the same. In some embodiments, the first IL-22 monomer and the second IL-22 monomer are different.

In some embodiments, the linking moiety L is a short polypeptide comprising about 3 to about 50 amino acids. In some embodiments, the L is a linker (e.g., peptide linker), such as any of the linkers described herein. In some embodiments, the L is peptide linker comprising (or consisting essentially of, or consisting of) the sequence of any one of SEQ ID NOs: 1-20 and 32. In some embodiments, the L is peptide linker of about 3 to about 50 amino acids in length. In some embodiments, the L is peptide linker of about 6 to about 30 amino acids in length. In some embodiments, the L is peptide linker comprising (or consisting essentially of, or consisting of) the sequence of SEQ ID NO: 1 or 10. In some embodiments, the first IL-22 monomer and the second IL-22 monomer are the same. In some embodiments, the first IL-22 monomer and the second IL-22 monomer are different. In some embodiments, the IL-22 monomer comprises (or consists essentially of, or consists of) the sequence of SEQ ID NO: 21. In some embodiments, the IL-22 dimer comprises (or consists essentially of, or consists of) the sequence of SEQ ID NO: 28. See FIG. 1 for an exemplary IL-22 dimer.

In some embodiments, the linking moiety L is a polypeptide of Formula II: —Z—Y—Z—; wherein Y is a carrier protein; Z is nothing, or a short peptide comprising about 1 to about 50 amino acids; and “-” is a chemical bond or a covalent bond. In some embodiments, “-” is a peptide bond. In some embodiments, Z is about 5 to about 50 amino acids in length. In some embodiments, Z is about 1 to about 30 amino acids in length. In some embodiments, Z is about 6 to about 30 amino acids in length. In some embodiments, Z comprises (or consists essentially of, or consists of) the sequence of any one of SEQ ID NOs: 1-20 and 32. In some embodiments, Z comprises (or consists essentially of, or consists of) the sequence of SEQ ID NO: 1 or 10. In some embodiments, the carrier protein comprises at least about two (such as 2, 3, 4, or more) cysteines capable of forming intermolecular disulfide bonds. In some embodiments, the carrier protein is N-terminal to the IL-22 monomer. In some embodiments, the carrier protein is C-terminal to the IL-22 monomer. In some embodiments, both IL-22 monomers are N-terminal to the carrier protein. See FIG. 2A as example. In some embodiments, both IL-22 monomers are C-terminal to the carrier protein. See FIG. 3A as example. In some embodiments, the carrier protein is an albumin (e.g., human albumin) or an Fc fragment of an immunoglobulin (such as IgG, e.g., human IgG). In some embodiments, the carrier protein is formed by the connection of two dimerization domains (e.g., two Fc fragments) via one or more disulfide bonds. In some embodiments, the first IL-22 monomer and the second IL-22 monomer are the same. In some embodiments, the first IL-22 monomer and the second IL-22 monomer are different.

In some embodiments, the IL-22 dimer comprises two monomeric subunits, wherein each monomeric subunit comprises an IL-22 monomer and a dimerization domain (e.g., Fc fragment). In some embodiments, the IL-22 monomer is connected to the dimerization domain via an optional linker. Thus in some embodiments, the IL-22 comprises two monomeric subunits, wherein each monomeric subunit comprises an IL-22 monomer, a dimerization domain (e.g., Fc fragment), and optionally a linker connecting the IL-22 monomer and the dimerization domain. In some embodiments, the dimerization domain (e.g., Fc fragment) comprises at least two (such as 2, 3, 4, or more) cysteines capable of forming intermolecular disulfide bonds (e.g., 2, 3, 4, or more disulfide bonds). In some embodiments, the dimerization domain comprises Fc fragment of human immunoglobulin (such as human IgG1, IgG2, IgG3, or IgG4), and the optional linker is a peptide linker connecting the IL-22 monomer and the Fc fragment, and the IL-22 dimer is formed by the connection of two dimerization domains (e.g., Fc fragment) via one or more disulfide bond(s). In some embodiments, the IL-22 monomer is N-terminal to the dimerization domain. In some embodiments, the IL-22 monomer is C-terminal to the dimerization domain. Thus in some embodiments, the IL-22 dimer comprises two monomeric subunits, wherein the first monomeric subunit comprises from N′ to C′: a first IL-22 monomer, a first optional linker, a first dimerization domain (e.g., Fc fragment); wherein the second monomeric subunit comprises from N′ to C′: a second IL-22 monomer, a second optional linker, a second dimerization domain (e.g., Fc fragment); and wherein the first monomeric subunit and the second monomeric subunit are connected via intermolecular disulfide bonds (e.g., 2, 3, 4, or more disulfide bonds) formed by two or more (such as 2, 3, 4, or more) cysteines of each dimerization domain. See FIG. 2B as example. In some embodiments, the IL-22 dimer comprises two monomeric subunits, wherein the first monomeric subunit comprises from N′ to C′: a first dimerization domain (e.g., Fc fragment), a first optional linker, a first IL-22 monomer; wherein the second monomeric subunit comprises from N′ to C′: a second dimerization domain (e.g., Fc fragment), a second optional linker, a second IL-22 monomer; and wherein the first monomeric subunit and the second monomeric subunit are connected via intermolecular disulfide bonds (e.g., 2, 3, 4, or more disulfide bonds) formed by two or more (such as 2, 3, 4, or more) cysteines of each dimerization domain. See FIG. 3B as example. In some embodiments, the first and second optional linkers are the same. In some embodiments, the first and second optional linkers are different. In some embodiments, one of the two monomeric subunits does not comprise a linker. In some embodiments, neither monomeric subunit comprises a linker. In some embodiments, both monomeric subunits comprise a linker. In some embodiments, the first IL-22 monomer and the second IL-22 monomer are the same. In some embodiments, the first IL-22 monomer and the second IL-22 monomer are different. In some embodiments, the first dimerization domain and the second dimerization domain are the same (e.g., both are IgG2 Fc). In some embodiments, the first dimerization domain and the second dimerization domain are different. In some embodiments, the dimerization domain comprises leucine zippers. In some embodiments, the dimerization domain comprises at least a portion of an Fc fragment (e.g., Fc fragment of IgG1, IgG2, IgG3, or IgG4). In some embodiments, the Fc fragment comprises CH2 and CH3 domains. In some embodiments, the Fc fragment is derived from IgG2, such as human IgG2. In some embodiments, the Fc fragment comprises (or consists essentially of, or consists of) the sequence of SEQ ID NO: 22 or 23. In some embodiments, the IL-22 monomer comprises (or consists essentially of, or consists of) the sequence of SEQ ID NO: 21. In some embodiments, the linker comprises (or consists essentially of, or consists of) the sequence of any one of SEQ ID NOs: 1-20 and 32. In some embodiments, the linker is about 1 to about 50 amino acids in length. In some embodiments, the linker is about 5 to about 50 amino acids in length. In some embodiments, the linker is about 1 to about 30 amino acids in length. In some embodiments, the linker is about 6 to about 30 amino acids in length. In some embodiments, the linker comprises (or consists essentially of, or consists of) the sequence of SEQ ID NO: 1 or 10. In some embodiments, each monomeric subunit comprises (or consists essentially of, or consists of) the sequence of any of SEQ ID NOs: 24-27. In some embodiments, each monomeric subunit comprises (or consists essentially of, or consists of) the sequence of SEQ ID NO: 24.

In some embodiments, the IL-22 dimer comprises two monomeric subunits, wherein each monomeric subunit comprises an IL-22 monomer and a dimerization domain. In some embodiments, the IL-22 monomer is fused to the N-terminus of the dimerization domain. In some embodiments, the IL-22 monomer is fused to the C-terminus of the dimerization domain. In some embodiments, the IL-22 monomer and the dimerization domain are linked via an optional peptide linker (e.g., a peptide linker of about 5 to about 50 amino acids in length, such as a linker comprising the sequence of SEQ ID NO: 1 or 10). In some embodiments, the dimerization domain comprises leucine zippers.

In some embodiments, the IL-22 dimer comprises two IL-22 monomeric subunits, wherein each monomeric subunit comprises an IL-22 monomer and at least a portion of an immunoglobulin Fc fragment (“Fc fragment”). In some embodiments, the IL-22 monomer is fused to the N-terminus of the Fc fragment. In some embodiments, the IL-22 monomer is fused to the C-terminus of the Fc fragment. In some embodiments, the IL-22 monomer and the Fc fragment are linked via an optional peptide linker (such as a peptide linker of about 5 to about 50 amino acids in length, e.g., a linker comprising the sequence of SEQ ID NO: 1 or 10). In some embodiments, the IL-22 monomer comprises (or consists essentially of, or consists of) the sequence of SEQ ID NO: 21. In some embodiments, the Fc fragment comprises at least two cysteines capable of forming intermolecular disulfide bonds. In some embodiments, the Fc fragment is truncated at the N-terminus, e.g., lacks the first 1, 2, 3, 4, 5, 6, 7, 8, 9, or 10 amino acids of a complete immunoglobulin Fc domain. In some embodiments, the Fc fragment is of type IgG2. In some embodiments, the Fc fragment is of type IgG4. In some embodiments, the Fc fragment comprises (or consists essentially of, or consists of) the sequence of SEQ ID NO: 22 or SEQ ID NO: 23.

In some embodiments, the IL-22 dimer comprises two monomeric subunits, wherein each monomeric subunit comprises (or consists essentially of, or consists of) the sequence of any of SEQ ID NOs: 24-27.

The amino acid sequence of an exemplary IL-22 dimer is shown in SEQ ID NO: 28, in which amino acid residues 1-146 represent the first IL-22 monomer, amino acid residues 147-162 represent the linker, and amino acid residues 163-308 represent the second IL-22 monomer. See FIG. 1 as example.

The amino acid sequence of an exemplary monomeric subunit comprising IL-22 monomer, linker, and Fc fragment, which is used to form an exemplary IL-22 dimer, is shown in SEQ ID NO: 24, in which amino acid residues 1-146 represent an IL-22 monomer, amino acid residues 147-162 represent the linker, and amino acid residues 163-385 represent Fc fragment of human IgG2. An IL-22 dimer is formed by the two monomeric subunits via the coupling of the Fc fragments. See FIG. 2B as example.

The amino acid sequence of an exemplary monomeric subunit comprising IL-22 monomer, linker, and Fc fragment, which is used to form an exemplary IL-22 dimer, is shown in SEQ ID NO: 26, in which amino acid residues 1-146 represent an IL-22 monomer, amino acid residues 147-152 represent the linker, and amino acid residues 153-375 represent Fc fragment of human IgG2. An IL-22 dimer is formed by the two monomeric subunits via the coupling of the Fc fragments. See FIG. 2B as example.

The amino acid sequence of an exemplary monomeric subunit comprising IL-22 monomer, linker, and Fc fragment, which is used to form an exemplary IL-22 dimer, is shown in SEQ ID NO: 25, in which amino acid residues 1-223 represent Fc fragment of human IgG2, amino acid residues 224-239 represent the linker, and amino acid residues 240-385 represent an IL-22 monomer. An IL-22 dimer is formed by the two monomeric subunits via the coupling of the Fc fragments. See FIG. 3B as example.

The amino acid sequence of an exemplary monomeric subunit comprising IL-22 monomer, linker, and Fc fragment, which is used to form an exemplary IL-22 dimer, is shown in SEQ ID NO: 27, in which amino acid residues 1-223 represent Fc fragment of human IgG2, amino acid residues 224-229 represent the linker, and amino acid residues 230-375 represent an IL-22 monomer. An IL-22 dimer is formed by the two monomeric subunits via the coupling of the Fc fragments. See FIG. 3B as example.

In some embodiments, an amino acid sequence not affecting the biological activity of IL-22 monomer and/or IL-22 dimer can be added to the N-terminus or C-terminus of the IL-22 dimer (or monomeric subunit thereof). In some embodiments, such appended amino acid sequence is beneficial to expression (e.g. signal peptide, such as SEQ ID NO: 30), purification (e.g., 6×His sequence, the cleavage site of Saccharomyces cerevisiae α-mating factor secretion signal leader (Glu-Lys-Arg; SEQ ID NO: 33)), or enhancement of biological activity of the IL-22 dimer.

The invention encompasses modifications to the polypeptides described herein, including functionally equivalent modifications which do not significantly affect their properties and variants which have enhanced or decreased activity. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, non-conservative mutations which do not significantly deleteriously change the functional activity, or use of chemical analogs.

Amino acid sequence insertions include amino- and/or carboxyl-terminal fusions ranging in length from one residue to polypeptides comprising a hundred or more residues, as well as intrasequence insertions of single or multiple amino acid residues. Examples of terminal insertions include an N-terminal methionyl residue or an epitope tag. Other insertional variants of the IL-22 monomeric subunits include fusion to the N-terminus or C-terminus of the polypeptide, or a polypeptide that increases the serum half-life of the IL-22 dimer.

Twenty amino acids are commonly found in proteins. Those amino acids can be grouped into nine classes or groups based on the chemical properties of their side chains. Substitution of one amino acid residue for another within the same class or group is referred to herein as a “conservative” substitution. Conservative amino acid substitutions can frequently be made in a protein without significantly altering the conformation or function of the protein. In contrast, non-conservative amino acid substitutions tend to disrupt conformation and function of a protein. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). See Table B.

TABLE B
Examples of amino acid classification

| | | | |
|---|---|---|---|
| Small/Aliphatic | Gly, Ala, | Basic Residues: | Lys, Arg |
| residues: | Val, Leu, Ile | | |
| Cyclic Imino Acid: | Pro | Imidazole Residue: | His |
| Hydroxyl-containing | Ser, Thr | Aromatic Residues: | Phe, Tyr, |
| Residues: | | | Trp |
| Acidic Residues: | Asp, Glu | Sulfur-containing | Met, Cys |
| | | Residues: | |
| Amide Residues: | Asn, Gln | | |

In some embodiments, the conservative amino acid substitution comprises substituting any of glycine (G), alanine (A), isoleucine (I), valine (V), and leucine (L) for any other of these aliphatic amino acids; serine (S) for threonine (T) and vice versa; aspartic acid (D) for glutamic acid (E) and vice versa; glutamine (Q) for asparagine (N) and vice versa; lysine (K) for arginine (R) and vice versa; phenylalanine (F), tyrosine (Y) and tryptophan (W) for any other of these aromatic amino acids; and methionine (M) for cysteine (C) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pKs of these two amino acid residues are not significant. Still other changes can be considered “conservative” in particular environments (see, e.g., Biochemistry at pp. 13-15, 2nd ed. Lubert Stryer ed. (Stanford University); Henikoff et al., Proc. Nat'l Acad. Sci. USA (1992) 89:10915-10919; Lei et al., J. Biol. Chem. (1995) 270(20):11882-11886).

In some embodiments, the IL-22 dimer described herein has an EC50 of no less than about 20 ng/mL (including for example no less than about any of 100 ng/mL, 200 ng/mL, 300 ng/mL, 400 ng/mL, or more) in an in vitro cell proliferation assay. In some embodiments, the IL-22 dimer has an EC50 that is at least about 5× (including for example at least about 10×, 30×, 50×, 100×, 150×, 300×, 400×, 500×, 600×, 1000× or more) that of a wildtype IL-22 monomer (for example the IL-22 monomer comprising the sequence of SEQ ID NO: 21) in an in vitro cell proliferation assay. In some embodiments, the IL-22 dimer has an EC50 of no less than about 10 ng/mL (including for example no less than about any of 50 ng/mL, 100 ng/mL, 200 ng/mL, 300 ng/mL, 400 ng/mL, or more) in an in vitro STAT3 stimulation assay. In some embodiments, the IL-22 dimer has an EC50 that is at least about 10× (including for example at least about 50×, 100×, 200×, 300×, 400×, 500×, 600×, 700×, 800×, 900×, 1000×, 1500×, or more) that of a wildtype IL-22 monomer (for example the IL-22 monomer comprising the sequence of SEQ ID NO: 21) in an in vitro STAT3 stimulation assay.

In some embodiments, the IL-22 dimer has a serum half-life that is significantly longer than that of IL-22. In some embodiments, the IL-22 dimer as a serum half-life of at least about any of 15, 30, 50, 100, 150, 200, 250, 300, or 350 hours. In some embodiments, while the dose of IL-22 dimer is about 2 μg/kg, the serum half-life is at least about any of 15, 30, 50, 100, 150, or 200 hours. In some embodiments, while the dose of IL-22 dimer is about 10 μg/kg, the serum half-life is at least about any of 50, 100, 150, or 200 hours. In some embodiments, while the dose of IL-22 dimer is about 30 μg/kg, the serum half-life is at least about any of 100, 150, 200, or 250 hours. In some embodiments, while the dose of IL-22 dimer is about 45 μg/kg, the serum half-life is at least about any of 100, 150, 200, 250, 300, or 350 hours.

In some embodiments, the IL-22 dimer retains the biological activity of IL-22 and has a longer serum half-life compared to that of the first and/or the second IL-22 monomer. In some embodiments, the serum half-life of the IL-22 dimer is at least about any of twice, 3, 4, 5, 6, 7, 8, 9, or 10 times longer than that of the first and/or the second IL-22 monomer.

IL-22 Monomer

Interleukin-22 (IL-22), also known as IL-10 related T cell-derived inducible factor (IL-TIF), is an α-helical cytokine. It belongs to a group of cytokines called the IL-10 family or IL-10 superfamily (including IL-19, IL-20, IL-24, and IL-26), which mediates cellular inflammatory responses. IL-22 is produced by several populations of immune cells, such as activated T cells (mainly CD4+ cells, especially CD28 pathway activated Tₕ1 cells, Tₕ17 cells, and Tₕ22 cells, etc.), IL-2/IL-12 stimulated natural killer cells (NK cells; Wolk et al., J. Immunology, 168:5379-5402, 2002), NK-T cells, neutrophils, and macrophages. Human IL-22 mRNA is mainly expressed in peripheral T cells upon stimulation by anti-CD3 antibodies or Concanavilin A (Con A). IL-22 can also be expressed in a number of organs and tissues upon lipopolysaccharide (LPS) stimulation, including gut, liver, stomach, kidney, lung, heart, thymus, and spleen, in which an increase of IL-22 expression can be measured (Dumoutier et al., PNAS. 2000). IL-22 binds to a heterodimeric cell surface receptor composed of IL-10R2 and IL-22R1 subunits. IL-22R1 is specific to IL-22 and is expressed mostly on non-hematopoietic cells, such as epithelial and stromal cells of liver, lung, skin, thymus, pancreas, kidney, gastrointestinal tract, synovial tissues, heart, breast, eye, and adipose tissue. The binding of IL-22 to IL-22R1/IL-10R2 receptor heterodimer activates intracellular kinases (JAK1, Tyk2, and MAP kinases) and transcription factors, especially STAT3.

Native human IL-22 precursor polypeptide consists of 179 amino acid residues (SEQ ID NO: 31), while the mature polypeptide consists of 146 amino acid residues (SEQ ID NO: 21). The human IL-22 signal peptide comprises the sequence of SEQ ID NO: 30. Dumoutier et al. first reported the cloned IL-22 DNA sequences of mouse and human (Dumoutier et al., Genes Immun. 2000; U.S. Pat. Nos. 6,359,117, and 6,274,710). Exemplary IL-22 polypeptide sequences are described in U.S. Patent Appln. No. US2003/0100076, U.S. Pat. Nos. 7,226,591, and 6,359,117, incorporated herein by reference in their entirety.

The terms “IL-22 polypeptide,” “IL-22,” “IL-22 molecule,” and “IL-22 protein” are used herein interchangeably. As used herein, the term “IL-22 monomer” refers to one unit of an IL-22 protein. In some embodiments, the IL-22 monomer is a full length IL-22. In some embodiments, the IL-22 monomer is an IL-22 functional fragment capable of producing most or full biological activity of a full length IL-22. In some embodiments, the IL-22 monomer is a precursor IL-22. In some embodiments, the IL-22 monomer is a mature IL-22. In some embodiments, the IL-22 monomer is a wild-type IL-22. In some embodiments, the IL-22 monomer is a mutant or variant IL-22, such as a mutant or variant IL-22 capable of producing most or full biological activity of a wild-type IL-22. In some embodiments, the IL-22 monomer is a modified IL-22, such as pegylated IL-22 and covalently modified IL-22 proteins. The IL-22 monomer described herein can be an IL-22 isolated from a variety of sources, such as from human tissue types or from another source, or prepared by recombinant or synthetic methods. In some embodiments, the IL-22 monomer is a recombinant IL-22. The IL-22 monomer described herein can be an IL-22 derived from any organism, such as mammals, including, but are not limited to, livestock animals (e.g., cows, sheep, goats, cats, dogs, donkeys, and horses), primates (e.g., human and non-human primates such as monkeys), rabbits, and rodents (e.g., mice, rats, gerbils, and hamsters). In some embodiments, the IL-22 monomer is a human IL-22 (hIL-22), such as recombinant human IL-22 (rhIL-22). In some embodiments, the IL-22 monomer is a murine IL-22 (mIL-22), such as recombinant murine IL-22 (rmIL-22). In some embodiments, the IL-22 monomer is a mature human IL-22, comprising the sequence of SEQ ID NO: 21. In some embodiments, the IL-22 monomer comprises a signal peptide at the N-terminal of the IL-22 protein, such as a signal peptide comprising the sequence of SEQ ID NO: 30. In some embodiments, the IL-22 monomer is a precursor human IL-22, comprising the sequence of SEQ ID NO: 31.

In some embodiments, the two IL-22 monomers forming the IL-22 dimer are the same (e.g., both comprise the sequence of SEQ ID NO: 21). In some embodiments, the two IL-22 monomers forming the IL-22 dimer are different, e.g., one IL-22 monomer is wild-type human IL-22 and one IL-22 monomer is mutated human IL-22.

Carrier Protein and Dimerization Domain

In some embodiments, the IL-22 dimer comprises two IL-22 monomers and a carrier protein. The carrier protein described herein can be any protein suitable for connecting two IL-22 monomers to form an IL-22 dimer, including but not limited to an Fc fragment of immunoglobulin (e.g., human IgG1, IgG2, IgG3, IgG4), or albumin (e.g., human serum albumin). When the carrier protein is formed by the connection of two protein subunits (e.g., via disulfide bond, peptide linkage, or chemical linkage), each protein subunit is referred to as a dimerization domain. In some embodiments, the carrier protein is formed by the connection of two dimerization domains (e.g., two Fc fragments of IgG) via one or more disulfide bonds. In some embodiments, the two dimerization domains forming the carrier protein are the same (e.g., two IgG2 Fc fragments). In some embodiments, the two dimerization domains forming the carrier protein are different. For example, in some embodiments, the carrier protein is formed by the connection of a first Fc fragment and a second different Fc fragment via one or more disulfide bonds. In some embodiments, the dimerization domain (e.g., Fc fragment) comprises at least two cysteines capable of forming intermolecular disulfide bonds. In some embodiments, there are about 2 to about 4 disulfide bonds between the two dimerization domains (e.g., Fc fragments). In some embodiments, the dimerization domain comprises leucine zippers. In some embodiments, the dimerization domain comprises at least a portion of an Fc fragment. In some embodiments, the Fc fragment comprises CH2 and CH3 domains. In some embodiments, the dimerization domain is derived from an Fc fragment of any of IgA, IgD, IgE, IgG, and IgM, and subtypes thereof. In some embodiments, the dimerization domain is derived from an Fc fragment of human IgG2. In some embodiments, the dimerization domain is derived from an Fc fragment of human IgG4. In some embodiments, the dimerization domain is a wild-type Fc fragment. In some embodiments, the dimerization domain comprises one or more mutations, such as a mutation in the Fc fragment to reduce or abolish effector functions, e.g., decreased antibody dependent cellular cytotoxicity (ADCC) or reduced binding to FcγR. In some embodiments, the dimerization domain is an IgG2 Fc fragment comprising a P107S mutation. In some embodiments, the dimerization domain comprises a full length Fc fragment. In some embodiments, the dimerization domain comprises an N-terminus truncated Fc fragment, such as truncated Fc fragment with less N-terminal cysteines in order to reduce disulfide bond mis-pairing during dimerization. In some embodiments, the Fc fragment is truncated at the N-terminus, e.g., lacks the first 1, 2, 3, 4, 5, 6, 7, 8, 9, or 10 amino acids of a complete immunoglobulin Fc domain. In some embodiments, the dimerization domain is an IgG2 Fc fragment with N-terminal “ERKCC” sequence (SEQ ID NO: 29) removed. In some embodiments, the Fc fragment comprises (or consists essentially of, or consists of) the sequence of SEQ ID NO: 22 or 23.

Linker

In some embodiments, the IL-22 dimer comprises two IL-22 monomers connected to each other via an optional linker (e.g., peptide linker, non-peptide linker). In some embodiments, the IL-22 monomer is connected to the carrier protein (e.g., albumin, or dimerization domain such as Fc fragment) via an optional linker (e.g., peptide linker, non-peptide linker). In some embodiments, both IL-22 monomers are connected to the carrier protein via linkers. In some embodiments, the first IL-22 monomer is connected to the carrier protein via a linker, the second IL-22 monomer is connected to the carrier protein without linker. In some embodiments, the first linker connecting the first IL-22 monomer and the carrier protein (or first dimerization domain) and the second linker connecting the second IL-22 monomer and the carrier protein (or second dimerization domain) are the same. In some embodiments, the first linker connecting the first IL-22 monomer and the carrier protein (or first dimerization domain) and the second linker connecting the second IL-22 monomer and the carrier protein (or second dimerization domain) are different. In general, a linker does not affect or significantly affect the proper fold and conformation formed by the configuration of the two IL-22 monomers.

The linkers can be peptide linkers of any length. In some embodiments, the peptide linker is from about 1 amino acid to about 10 amino acids long, from about 2 amino acids to about 15 amino acids long, from about 3 amino acids to about 12 amino acids long, from about 4 amino acids to about 10 amino acids long, from about 5 amino acids to about 9 amino acids long, from about 6 amino acids to about 8 amino acids long, from about 1 amino acid to about 20 amino acids long, from about 21 amino acids to about 30 amino acids long, from about 1 amino acid to about 30 amino acids long, from about 2 amino acids to about 20 amino acids long, from about 10 amino acids to about 30 amino acids long, from about 3 amino acid to about 50 amino acids long, from about 2 amino acids to about 19 amino acids long, from about 2 amino acids to about 18 amino acids long, from about 2 amino acids to about 17 amino acids long, from about 2 amino acids to about 16 amino acids long, from about 2 amino acids to about 10 amino acids long, from about 2 amino acids to about 14 amino acids long, from about 2 amino acids to about 13 amino acids long, from about 2 amino acids to about 12 amino acids long, from about 2 amino acids to about 11 amino acids long, from about 2 amino acids to about 9 amino acids long, from about 2 amino acids to about 8 amino acids long, from about 2 amino acids to about 7 amino acids long, from about 2 amino acids to about 6 amino acids long, from about 2 amino acids to about 5 amino acids long, or from about 6 amino acids to about 30 amino acids long. In some embodiments, the peptide linker is about any of 1, 2, 3, 4, 5, 6, 7, 8, 9, 10, 11, 12, 13, 14, 15, 16, 17, 18, 19 or 20 amino acids long. In some embodiments, the peptide linker is about any of 21, 22, 23, 24, 25, 26, 27, 28, 29, or 30 amino acids long. In some embodiments, the peptide linker is about any of 31, 32, 33, 34, 35, 36, 37, 38, 39, 40, 41, 42, 43, 44, 45, 46, 47, 48, 49, or 50 amino acids long. For example, in some embodiments, the linker is about 1 to about 50 amino acids in length. In some embodiments, the linker is about 5 to about 50 amino acids in length. In some embodiments, the linker is about 6 to about 30 amino acids in length. In some embodiments, the linker is about 6 amino acids in length. In some embodiments, the linker is about 16 amino acids in length.

In some embodiments, the N-terminus of the peptide linker is covalently linked to the C-terminus of the IL-22 monomer, and the C-terminus of the peptide linker is covalently linked to the carrier protein (or the N-terminus of the dimerization domain). In some embodiments, the C-terminus of the peptide linker is covalently linked to the N-terminus of the IL-22 monomer, and the N-terminus of the peptide linker is covalently linked to the carrier protein (or the C-terminus of the dimerization domain).

A peptide linker can have a naturally occurring sequence or a non-naturally occurring sequence. For example, a sequence derived from the hinge region of a heavy chain only antibody can be used as a linker. See, for example, WO1996/34103. In some embodiments, the peptide linker is a human IgG1, IgG2, IgG3, or IgG4 hinge. In some embodiments, the peptide linker is a mutated human IgG1, IgG2, IgG3, or IgG4 hinge. In some embodiments, the linker is a flexible linker. Exemplary flexible linkers include, but are not limited to, glycine polymers (G)ₙ (SEQ ID NO: 6), glycine-serine polymers (including, for example, (GS)ₙ (SEQ ID NO: 7), (GSGGS)ₙ (SEQ ID NO: 8), (GGGS)ₙ (SEQ ID NO: 9), or (GGGGS)ₙ (SEQ ID NO: 11), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11 173-142 (1992)). Exemplary flexible linkers include, but are not limited to Gly-Gly (SEQ ID NO: 12), Gly-Gly-Ser-Gly (SEQ ID NO: 13), Gly-Gly-Ser-Gly-Gly (SEQ ID NO: 14), Gly-Ser-Gly-Ser-Gly (SEQ ID NO: 15), Gly-Ser-Gly-Gly-Gly (SEQ ID NO: 16), Gly-Gly-Gly-Ser-Gly (SEQ ID NO: 17), Gly-Ser-Ser-Ser-Gly (SEQ ID NO: 18), Gly-Gly-Ser-Gly-Gly-Ser (SEQ ID NO: 2), Ser-Gly-Gly-Gly-Gly-Ser (SEQ ID NO: 3), Gly-Arg-Ala-Gly-Gly-Gly-Gly-Ala-Gly-Gly-Gly-Gly (SEQ ID NO: 4), Gly-Arg-Ala-Gly-Gly-Gly (SEQ ID NO: 5), GGGGSGGGGSGGGGS (SEQ ID NO: 19), GGGGS (SEQ ID NO: 20), and the like. In some embodiments, the linker comprises (or consists essentially of, or consists of) the sequence of ASTKGP (SEQ ID NO: 10). In some embodiments, the linker comprises (or consists essentially of, or consists of) the sequence of GSGGGSGGGGSGGGGS (SEQ ID NO: 1). The ordinarily skilled artisan will recognize that design of an IL-22 dimer can include linkers that are all or partially flexible, such that the linker can include a flexible linker portion as well as one or more portions that confer less flexible structure to provide a desired IL-22 dimer structure and function.

In some embodiments, the linker between the IL-22 monomer and the carrier protein (e.g., dimerization domain) is a stable linker (not cleavable by protease, especially MMPs).

In some embodiments, the linker comprises an amino acid sequence selected from any of: (a) an amino acid sequence comprising (or consisting essentially of, or consisting of) about 3 to about 16 hydrophobic amino acid residues Gly or Pro, such as Gly-Pro-Gly-Pro-Gly-Pro (SEQ ID NO: 32); (b) an amino acid sequence encoded by multiple cloning sites (MCS), usually about 5 to about 20 amino acid residues long, or about 10 to about 20 amino acid residues long; (c) an amino acid sequence of a polypeptide other than IL-22 monomer, such as an amino acid sequence of IgG or albumin; and (d) an amino acid sequence comprising any combination of (a), (b), and (c).

Any one or all of the linkers described herein can be accomplished by any chemical reaction that will bind the two IL-22 monomers or the IL-22 monomer and the carrier protein (or dimerization domain) so long as the components or fragments retain their respective activities, i.e. binding to IL-22 receptor, binding to FcR, or ADCC. This linkage can include many chemical mechanisms, for instance covalent binding, affinity binding, intercalation, coordinate binding and complexation. In some embodiments, the binding is covalent binding. Covalent binding can be achieved either by direct condensation of existing side chains or by the incorporation of external bridging molecules. Many bivalent or polyvalent linking agents are useful in coupling protein molecules, such as an Fc fragment to IL-22 monomer of the present invention. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents (see Killen and Lindstrom, Jour. Immun. 133:1335-2549 (1984); Jansen et al., Immunological Reviews 62:185-216 (1982); and Vitetta et al., Science 238:1098 (1987)).

Linkers that can be applied in the present application are described in the literature (see, for example, Ramakrishnan, S. et al., Cancer Res. 44:201-208 (1984) describing use of MBS (M-maleimidobenzoyl-N-hydroxysuccinimide ester)). In some embodiments, non-peptide linkers used herein include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido] hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above can contain components that have different attributes, thus leading to IL-22 dimers with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form fusion protein with increased stability. Disulfide linkages, are in general, less stable than other linkages because the disulfide linkage is cleaved in vitro, resulting in less fusion protein available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

Other linker considerations include the effect on physical or pharmacokinetic properties of the resulting IL-22 dimer, such as solubility, lipophilicity, hydrophilicity, hydrophobicity, stability (more or less stable as well as planned degradation), rigidity, flexibility, immunogenicity, modulation of IL-22/IL-22 receptor binding, the ability to be incorporated into a micelle or liposome, and the like.

Biological Activities

In some embodiments, the biological activity of the IL-22 dimer described herein is selected from one or more of: (a) reducing the levels of amylase, lipase, TG, AST, and/or ALT in vivo, such as reducing at least about 10% (including for example at least about any of 20%, 30%, 40%, 50%, 60%, 70%, 80%, 90%, or 100%); (b) controlling, ameliorating, and/or preventing tissue and/or organ (e.g., lung, heart, kidney, liver) injury or failure (e.g., pulmonary fibrosis) in vivo, such as induced by virus infection; (c) controlling, reducing, and/or inhibiting cell necrosis in vitro and/or in vivo (such as reducing at least about 10% (including for example at least about any of 20%, 30%, 40%, 60%, 70%, 80%, 90%, or 100%) cell necrosis), such as necrosis in infected and/or non-infected tissue and/or organ (e.g., lung, heart, kidney, liver); (d) controlling, ameliorating, and/or preventing the infiltration of inflammatory cells (e.g., NK cell, CTL, neutrophil, monocyte, macrophage) in tissues and/or organs (infected or non-infected) in vitro and/or in vivo, such as reducing at least about 10% (including for example at least about any of 20%, 30%, 40%, 50%, 60%, 70%, 80%, 90%, or 100%) inflammatory cell infiltration; (e) controlling, ameliorating and/or preventing inflammation in infected or non-infected tissue and/or organ, systemic inflammation, and/or cytokine storm, e.g., changing serum levels of inflammatory markers such as IL-6, IL-8, IL-10, IL1B, IL-12, IL-15, IL-17, CCL2, IL-1α, IL-2, IL-5, IL-9, CCL4, M-CSF, MCP-1, GCSF, MIP1A, CRP, TNFα, TNFβ, IFNγ, IP10, and MCP1, such as downregulating at least about 10% (including for example at least about any of 20%, 30%, 40%, 50%, 60%, 70%, 80%, 90%, or 100%), or down-regulating (e.g., downregulating at least about any of 5%, 10%, 20%, 30%, 40%, 50%, 60%, 70%, 80%, 90%, or more) pro-inflammatory pathways such as TLR4 signaling; (f) promoting tissue and/or organ regeneration, such as upregulating regeneration markers such as ANGPT2, FGF-b, PDGF-AA, Reg3A, and PDGF-BB (e.g., upregulating at least about 10% (including for example at least about any of 20%, 30%, 40%, 50%, 60%, 70%, 80%, 90%, or 100%)); (g) protecting tissue and/or organ (e.g., lung, heart, kidney, liver) from adverse effects (e.g., injury) triggered by additional therapy, such as antiviral drugs; (h) decreasing ARDS score for viral infection associated with respiratory system (e.g., lung); (i) controlling, ameliorating, and/or preventing sepsis, SIRS, septic shock, and/or MODS; (j) reducing mortality rate associated with virus infection, and/or preventing death, such as reducing at least about 10% (including for example at least about any of 20%, 30%, 40%, 50%, 60%, 70%, 80%, 90%, or 100%) death rate; (k) decreasing Acute Physiology And Chronic Health Evaluation II (APACHE II) score or KNAUS score (for MODS) in an individual; (l) improving organ function test scores (e.g., lung function test score); (m) treating or preventing metabolic disease, fatty liver, hepatitis, sepsis, MODS, neurological disorder, and pancreatitis associated with viral infection; (n) increasing point (e.g., greater than or equal to 2-point increase) in the NIAID 8-point ordinal scale; (o) reducing length of hospital stay (e.g., reducing at least about any of 1, 2, 3, 4, 5, 10, 20, 30, 60, 90, 120, 180, or more days of hospital stay); (p) increasing alive and respiratory failure free days (e.g., increasing at least about any of 1, 2, 3, 4, 5, 10, 20, 30, 60, 90, 120, 180, or more days); (q) controlling, ameliorating, and/or preventing progression to severe/critical disease (e.g., reducing or preventing at least about any of 5%, 10%, 20%, 30%, 40%, 50%, 60%, 70%, 80%, 90%, or more severe progression); (r) controlling, reducing, and/or preventing occurrence of any new infections (e.g., reducing or preventing at least about any of 5%, 10%, 20%, 30%, 40%, 50%, 60%, 70%, 80%, 90%, or more new infections); (s) controlling, ameliorating, and/or preventing endothelial (e.g., pulmonary endothelial) dysfunction, injury, or death (e.g., reducing or preventing at least about any of 5%, 10%, 20%, 30%, 40%, 50%, 60%, 70%, 80%, 90%, or more endothelial dysfunction, injury, or death); (t) controlling, ameliorating, and/or preventing (e.g., reducing or preventing at least about any of 5%, 10%, 20%, 30%, 40%, 50%, 60%, 70%, 80%, 90%, or more) damage and/or degradation of EGX, endothelial cell surface proteins, and/or adherens junctions between endothelial cells, such as by down-regulating (e.g., down-regulating at least about any of 5%, 10%, 20%, 30%, 40%, 50%, 60%, 70%, 80%, 90%, or more) extracellular proteinase (e.g., MMPs) expression and/or up-regulating (e.g., up-regulating at least about any of 5%, 10%, 20%, 30%, 40%, 50%, 60%, 70%, 80%, 90%, or more) extracellular matrix protein expression (e.g., Tnc, collagen, type I, COL1a1, collagen, type VI, Col6a3, and collagen, type I, Col1a2); (u) controlling, ameliorating, and/or preventing (e.g., reducing or preventing at least about any of 5%, 10%, 20%, 30%, 40%, 50%, 60%, 70%, 80%, 90%, or more) protein leakage; (v) promoting regeneration of EGX and/or endothelial (e.g., pulmonary endothelial) cells, such as increasing at least about any of 5%, 10%, 20%, 30%, 40%, 50%, 60%, 70%, 80%, 90%, or more functional EGX and/or endothelial cells; (w) reducing (e.g., at least about any of 5%, 10%, 20%, 30%, 40%, 50%, 60%, 70%, 80%, 90%, or more) viral load in infected tissue and/or organ; and (x) reducing or preventing (e.g., at least about any of 5%, 10%, 20%, 30%, 40%, 50%, 60%, 70%, 80%, 90%, or more) organ (e.g., lung) collagen deposition.

In some embodiments, the IL-22 dimer treatment of said viral infection controls and/or attenuates and/or inhibits a cytokine storm induced by said viral pathogen. In some embodiments, said treatment prevents worsening, arrests and/or ameliorates at least one symptom of said viral infection or damage to said subject or an organ or tissue of said subject, emanating from or associated with said viral infection. The symptom or damage emanating from or associated with said viral infection can be, but are not limited to, gastrointestinal symptoms such as diarrhea, fever (e.g., body temperature of >38° C.), kidney failure, heart failure, liver failure, respiratory symptoms such as cough, pulmonary fibrosis, pneumonia, shortness of breath, breathing difficulties, respiratory failure, shock, acute respiratory distress syndrome (ARDS), systemic inflammatory response syndrome (SIRS), multiple organ dysfunction syndrome (MODS), hypotension, tachycardia, dyspnea, ischemia, insufficient tissue perfusion (especially involving the major organs such as heart, liver, lung, kidney), uncontrollable hemorrhage, multisystem organ failure (primarily due to hypoxia or tissue acidosis) or severe metabolism dysregulation. In some embodiments, the IL-22 dimer treatment described herein prevents death of said virus-infected subject.

### Dosage Regimen and Routes of Administration of IL-22 Dimer

The IL-22 dimer described herein (or pharmaceutical composition thereof) is administered in an effective amount to treat a disease or disorder (e.g., virus-induced organ injury or failure) in a virus-infected subject, such as achieving one or more of the desired treatment effects or biological functions described herein.

Suitable dosage of the IL-22 dimer (or pharmaceutical composition thereof) described herein includes, for example, about 2 μg/kg to about 200 μg/kg, including for example about 2 μg/kg to about 100 μg/kg, about 5 μg/kg to about 80 μg/kg, about 5 μg/kg to about 50 μg/kg, about 10 μg/kg to about 45 μg/kg, about 10 μg/kg to about 30 μg/kg, about 30 to about 45 μg/kg, or about 30 to about 40 μg/kg. In some embodiments, the IL-22 dimer is administered (e.g., intravenously) at the dose of at least about any of 0.01 μg/kg, 0.05 μg/kg, 0.1 μg/kg, 0.5 μg/kg, 1 μg/kg, 2 μg/kg, 5 μg/kg, 10 μg/kg, 15 μg/kg, 20 μg/kg, 25 μg/kg, 30 μg/kg, 40 μg/kg, 45 μg/kg, 50 μg/kg, 60 μg/kg, 70 μg/kg, 80 μg/kg, 90 μg/kg, 100 μg/kg, 150 μg/kg, 200 μg/kg, 250 μg/kg, 300 μg/kg, 400 μg/kg, 500 μg/kg, 600 μg/kg, 700 μg/kg, 800 μg/kg, 900 μg/kg, or 1 mg/kg. In some embodiments, the IL-22 dimer is administered (e.g., intravenously) at the dose of no more than about any of 0.01 μg/kg, 0.05 μg/kg, 0.1 μg/kg, 0.5 μg/kg, 1 μg/kg, 2 μg/kg, 5 μg/kg, 10 μg/kg, 15 μg/kg, 20 μg/kg, 25 μg/kg, 30 μg/kg, 40 μg/kg, 45 μg/kg, 50 μg/kg, 60 μg/kg, 70 μg/kg, 80 μg/kg, 90 μg/kg, 100 μg/kg, 150 μg/kg, 200 μg/kg, 250 μg/kg, 300 μg/kg, 400 μg/kg, 500 μg/kg, 600 μg/kg, 700 μg/kg, 800 μg/kg, 900 μg/kg, or 1 mg/kg. The doses described herein may refer to a suitable dose for cynomolgus monkeys, a mouse equivalent dose thereof, a human equivalent dose thereof, or an equivalent dose for the specific species of the individual. In some embodiments, the IL-22 dimer is administered intravenously at the dose of at least about any of 10 μg/kg, 20 μg/kg, 30 μg/kg, 40 μg/kg, 45 μg/kg, or 50 μg/kg. In some embodiments, the IL-22 dimer is administered intravenously at the dose of no more than about any of 10 μg/kg, 20 μg/kg, 30 μg/kg, 40 μg/kg, 45 μg/kg, or 50 μg/kg. In some embodiments, the effective amount of the IL-22 dimer is about 2 μg/kg to about 200 μg/kg. In some embodiments, the effective amount of the IL-22 dimer is about 5 μg/kg to about 80 μg/kg. In some embodiments, the effective amount of the IL-22 dimer is about 10 μg/kg to about 45 μg/kg. In some embodiments, the effective amount of the IL-22 dimer is about 10 μg/kg to about 15 μg/kg, about 15 μg/kg to about 20 μg/kg, about 20 μg/kg to about 25 μg/kg, about 25 μg/kg to about 30 μg/kg, or about 30 μg/kg to about 45 μg/kg. In some embodiments, the IL-22 dimer is administered at about 20 μg/kg to about 40 μg/kg, including for example about 30 μg/kg to about 35 μg/kg.

The effective amount of the IL-22 dimer (or pharmaceutical composition thereof) may be administered in a single dose or in multiple doses. For methods that comprises administration of the IL-22 dimer in multiple doses, exemplary dosing frequencies include, but are not limited to, daily, daily without break, weekly, weekly without break, weekly for two out of three weeks, weekly for three out of four weeks, once every three weeks, once every two weeks, monthly, every six months, yearly, etc. In some embodiments, the IL-22 dimer is administered about once every 2 weeks, once every 3 weeks, once every 4 weeks, once every 6 weeks, or once every 8 weeks. In some embodiments, the IL-22 dimer is administered at least about any of 1×, 2×, 3×, 4×, 5×, 6×, or 7× (i.e., daily) a week. In some embodiments, the IL-22 dimer is administered no more than about once every 2, 3, 4, 5, 6, or 7 years. In some embodiments, the intervals between each administration are less than about any of 3 years, 2 years, 12 months, 11 months, 10 months, 9 months, 8 months, 7 months, 6 months, 5 months, 4 months, 3 months, 2 months, 1 month, 4 weeks, 3 weeks, 2 weeks, 1 week, 6 days, 5 days, 4 days, 3 days, 2 days, or 1 day. In some embodiments, the intervals between each administration are more than about any of 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, 12 months, 2 years, or 3 years. In some embodiments, there is no break in the dosing schedule.

The administration of the IL-22 dimer (or pharmaceutical composition thereof) can be extended over an extended period of time, such as from 1 day to about a week, from about a week to about a month, from about a month to about a year, from about a year to about several years. In some embodiments, the IL-22 dimer is administered over a period of at least any of about 1 day, 2 days, 3 days, 4 days, 5 days, 6 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more.

In some embodiments, the IL-22 dimer described herein (or pharmaceutical composition thereof) is administered once every week. In some embodiments, the IL-22 dimer described herein (or pharmaceutical composition thereof) is administered twice every week. In some embodiments, the IL-22 dimer is administered once every 2, 3, 4, 5, 6, 7, 8, 9, 10, 11, 12, or 24 weeks. In some embodiments, the IL-22 dimer is administered once every 1, 2, 3, 4, 5, 6, 7, 8, 9, 10, or 12 months. In some embodiments, the IL-22 dimer is administered only once. In some embodiments, the IL-22 dimer is administered no more frequently than once every week, once every month, once every two months, or once every six months. In some embodiments, the IL-22 dimer is administered at least once a week. In some embodiments, the IL-22 dimer is administered on day 1 and day 6 of a 10-day treatment cycle. In some embodiments, the IL-22 dimer is administered on day 1 and day 8 of a 14-day treatment cycle.

The IL-22 dimer described herein (or pharmaceutical composition thereof) can be administered via a variety of modes of administration suitable for treating the specific type of virus-induced disorder (e.g., injury or failure of lung, heart, kidney, liver, sepsis, septic shock, or MODS), including for example systemic or localized administration, depending on whether local or systemic treatment is desired and upon the area to be treated. In some embodiments, the IL-22 dimer is administered enternally. In some embodiments, the IL-22 dimer is administered parenterally (e.g. by injection, either subcutaneously, intraperitoneally, intravenously, or intramuscularly, or delivered to the interstitial space of a tissue). In some embodiments, the IL-22 dimer is administered intravenously, such as via IV push, IV infusion, or continuous IV infusion. In some embodiments, the IL-22 dimer is administered subcutaneously. In some embodiments, the IL-22 dimer is administered locally, such as intrapulmonarily or intracardialy. In some embodiments, the IL-22 dimer is administered via inhalation or insufflation, such as through mouth or nose. In some embodiments, the IL-22 dimer is delivered nasally, by inhalation, for example, using a metered-dose inhaler, nebuliser, dry powder inhaler, or nasal inhaler. In some embodiments, administration can also be topical (including ophthalmic and to mucous membranes including vaginal and rectal delivery). In some embodiments, the IL-22 dimer is administered into a lesion. Other modes of administration include oral and pulmonary administration, suppositories, and transdermal or transcutaneous applications, needles, and hyposprays.

### Pharmaceutical Compositions, Unit Dosages, Articles of Manufacture, and Kits

In some embodiments, the IL-22 dimer is formulated into a pharmaceutical composition comprising any of the IL-22 dimer described herein, and optionally a pharmaceutically acceptable carrier.

The pharmaceutical compositions may be suitable for a variety of modes of administration described herein, including for example systemic or localized administration. In some embodiments, the pharmaceutical composition is formulated for intravenous administration. In some embodiments, the pharmaceutical composition is formulated for subcutaneous administration. In some embodiments, the pharmaceutical composition is formulated for local administration, such as to lung, heart, kidney, liver, etc. In some embodiments, the pharmaceutical composition is formulated for inhalation or insufflation, such as through mouth or nose (e.g., powders or aerosols), including by nebulizer. In some embodiments, the pharmaceutical composition is formulated for topical administration. In some embodiments, the pharmaceutical composition is formulated for oral or pulmonary administration, suppositories, and transdermal or transcutaneous applications, needles, and hyposprays

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some embodiments, the pharmaceutical composition is formulated to have a pH in the range of about 4.5 to about 9.0, including for example pH ranges of about any one of 5.0 to about 8.0, about 6.5 to about 7.5, or about 6.5 to about 7.0. In some embodiments, the pharmaceutical composition can also be made to be isotonic with blood by the addition of a suitable tonicity modifier, such as glycerol.

The pharmaceutical compositions to be used for in vivo administration are generally formulated as sterile, substantially isotonic, and in full compliance with all Good Manufacturing Practice (GMP) regulations of the U.S. Food and Drug Administration. Sterility is readily accomplished by filtration through sterile filtration membranes. In some embodiments, the composition is free of pathogen. For injection, the pharmaceutical composition can be in the form of liquid solutions, for example in physiologically compatible buffers such as Hank's solution or Ringer's solution. In addition, the pharmaceutical composition can be in a solid form and re-dissolved or suspended immediately prior to use. Lyophilized compositions are also included.

In some embodiment, the pharmaceutical composition is formulated in accordance with routine procedures as a pharmaceutical composition adapted for injection intravenously, introperitoneally, or intravitreally. Typically, compositions for injection are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachett indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Formulations suitable for intrapulmonary or nasal administration have a particle size for example in the range of 0.1 to 500 microns, such as 0.5, 1, 30, 35 etc., which is administered by rapid inhalation through the nasal passage or by inhalation through the mouth so as to reach the alveolar sacs. Suitable formulations include aqueous or oily solutions of the IL-22 dimer. Formulations suitable for aerosol or dry powder administration may be prepared according to conventional methods.

In some embodiments, the pharmaceutical composition is suitable for administration to a human. In some embodiments, the pharmaceutical composition is suitable for administration to a rodent (e.g., mice, rats) or non-human primates (e.g., Cynomolgus monkey). In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

Also provided are unit dosage forms of the IL-22 dimer described herein, or compositions (such as pharmaceutical compositions) thereof. The term “unit dosage form” refers to a physically discrete unit suitable as unitary dosages for an individual, each unit containing a predetermined quantity of active material calculated to produce the desired therapeutic effect, in association with a suitable pharmaceutical carrier, diluent, or excipient. These unit dosage forms can be stored in a suitable packaging in single or multiple unit dosages and may also be further sterilized and sealed.

The present application further provides articles of manufacture comprising the IL-22 dimer compositions (or pharmaceutical composition thereof) described herein in suitable packaging. Suitable packaging for IL-22 dimer compositions (such as pharmaceutical compositions) described herein are known in the art, and include, for example, vials (such as sealed vials), vessels, ampules, bottles, IV bags, jars, inhaler, flexible packaging (e.g., sealed Mylar or plastic bags), and the like. These articles of manufacture may further be sterilized and/or sealed.

The present application also provides kits comprising IL-22 dimer compositions (such as pharmaceutical compositions) described herein and may further comprise instruction(s) on methods of using the composition, such as uses described herein. The kits described herein may further include other materials desirable from a commercial and user standpoint, including other buffers, diluents, filters, needles, syringes, and package inserts with instructions for performing any methods described herein.

For example, in some embodiments, there is provided a kit comprising an IL-22 dimer and an instruction for administering the IL-22 dimer intravenously, for example at a dosage of about 2 μg/kg to about 200 μg/kg (such as about 10 μg/kg to about 45 μg/kg). In some embodiments, there is provided a unit dosage form for intravenous or intrapulmonary administration or for inhalation or insufflation, wherein the unit dosage form comprises an effective amount of IL-22 dimer that would allow administration of the IL-22 dimer at a dosage of about 2 μg/kg to about 200 μg/kg (such as about 10 μg/kg to about 45 μg/kg). In some embodiments, there is provided a medicine comprising IL-22 dimer for intravenous or intrapulmonary administration or for inhalation or insufflation, wherein the medicine comprises an effective amount of IL-22 dimer that would allow administration of the IL-22 dimer at a dosage of about 2 μg/kg to about 200 μg/kg (such as about 10 μg/kg to about 45 μg/kg). In some embodiments, there is provided a use of IL-22 dimer for the manufacture of a medicament for treating a disease (e.g., preventing or treating organ injury or failure), wherein the medicament is suitable for intravenous or intrapulmonary administration or for inhalation or insufflation, and wherein the medicament comprises an effective amount of IL-22 dimer that would allow administration of IL-22 at a dosage of about 2 μg/kg to about 200 μg/kg (such as about 10 μg/kg to about 45 μg/kg).

### Combination Therapy

In some embodiments, the IL-22 dimer described herein can be administered in combination with a second therapy (e.g., surgery, a second therapeutic agent). In some embodiments, the IL-22 dimer described herein is administered in combination with an effective amount of another therapeutic agent.

For the treatment of virus-induced organ injury or failure, the other therapeutic agent can be active against viruses, such as against the particular pathogenic virus that causes the organ injury or failure. For respiratory infections, injuries, or failures, additional active therapeutics used to treat respiratory symptoms and sequelae of infection may be used, such as orally or by direct inhalation. In some embodiments, bronchodilators and corticosteroids can be used for combination therapy.

In some embodiments, the other therapeutic agent is selected from the group consisting of a corticosteroid, an anti-inflammatory signal transduction modulator, a 02-adrenoreceptor agonist bronchodilator, an anticholinergic, a mucolytic agent, an antiviral agent, an anti-fibrotic agent, hypertonic saline, an antibody, a vaccine, or mixtures thereof.

Glucocorticoids, which were first introduced as an asthma therapy in 1950 (Carryer, Journal of Allergy, 21, 282-287, 1950), remain the most potent and consistently effective therapy for this disease, although their mechanism of action is not yet fully understood (Morris, J. Allergy Clin. Immunol., 75 (1 Pt) 1-13, 1985). Unfortunately, oral glucocorticoid therapies are associated with profound undesirable side effects such as truncal obesity, hypertension, glaucoma, glucose intolerance, acceleration of cataract formation, bone mineral loss, and psychological effects, all of which limit their use as long-term therapeutic agents (Goodman and Gilman, 10th edition, 2001). A solution to systemic side effects is to deliver steroid drugs directly to the site of inflammation. Inhaled corticosteroids (ICS) have been developed to mitigate the severe adverse effects of oral steroids. Non-limiting examples of corticosteroids that may be used in combinations with the IL-22 dimer described herein are dexamethasone, dexamethasone sodium phosphate, fluorometholone, fluorometholone acetate, loteprednol, loteprednol etabonate, hydrocortisone, prednisolone, fludrocortisones, triamcinolone, triamcinolone acetonide, betamethasone, beclomethasone diproprionate, methylprednisolone, fluocinolone, fluocinolone acetonide, flunisolide, fluocortin-21-butylate, flumethasone, flumetasone pivalate, budesonide, halobetasol propionate, mometasone furoate, fluticasone propionate, ciclesonide; or a pharmaceutically acceptable salts thereof.

Other anti-inflammatory agents working through anti-inflamatory cascade mechanisms are also useful as additional therapeutic agents in combination with the IL-22 dimer described herein for the treatment of virus-induced organ injury or failure (e.g., viral respiratory infections). Applying “anti-inflammatory signal transduction modulators” (herein referred as AISTM), like phosphodiesterase inhibitors (e.g. PDE-4, PDE-5, or PDE-7 specific), transcription factor inhibitors (e.g. blocking NFxB through IKK inhibition), or kinase inhibitors (e.g. blocking P38 MAP, INK, PI3K, EGFR or Syk) is a logical approach to switching off inflammation as these small molecules target a limited number of common intracellular pathways—those signal transduction pathways that are critical points for the anti-inflammatory therapeutic intervention (see review by P. J. Barnes, 2006). These non-limiting additional therapeutic agents include: acalabrutinib (Calquence®); baricitinib (Olumiant®); ruxolitinib (Jakafi®); tofacitinib (Xeljanz®); 5-(2,4-Difluoro-phenoxy)-1-isobutyl-1H-indazole-6-carboxylic acid (2-dimethylamino-ethyl)-amide (P38 Map kinase inhibitor ARRY-797); 3-Cyclopropylmethoxy-N-(3,5-dichloro-pyridin-4-yl)-4-difluorormethoxy-benzamide (PDE-4 inhibitor Roflumilast); 4-[2-(3-cyclopentyloxy-4-methoxyphenyl)-2-phenyl-ethyl]-pyridine (PDE-4 inhibitor CDP-840); N-(3,5-dichloro-4-pyridinyl)-4-(difluoromethoxy)-8-[(methylsulfonyl)amino]-1-dibenzofurancarboxamide (PDE-4 inhibitor Oglemilast); N-(3,5-Dichloro-pyridin-4-yl)-2-[1-(4-fluorobenzyl)-5-hydroxy-1H-indol-3-yl]-2-oxo-acetamide (PDE-4 inhibitor AWD 12-281); 8-Methoxy-2-trifluoromethyl-quinoline-5-carboxylic acid (3,5-dichloro-1-oxy-pyridin-4-yl)-amide (PDE-4 inhibitor Sch 351591); 4-[5-(4-Fluorophenyl)-2-(4-methanesulfinyl-phenyl)-1H-imidazol-4-yl]-pyridine (P38 inhibitor SB-203850); 4-[4-(4-Fluoro-phenyl)-1-(3-phenyl-propyl)-5-pyridin-4-yl-1H-imidazol-2-yl]-but-3-yn-1-ol (P38 inhibitor RWJ-67657); 4-Cyano-4-(3-cyclopentyloxy-4-methoxy-phenyl)-cyclohexanecarboxylic acid 2-diethyl amino-ethyl ester (2-diethyl-ethyl ester prodrug of Cilomilast, PDE-4 inhibitor); (3-Chloro-4-fluorophenyl)-[7-methoxy-6-(3-morpholin-4-yl-propoxy)-quinazolin-4-yl]-amine (Gefitinib, EGFR inhibitor); and 4-(4-Methyl-piperazin-1-ylmethyl)-N-[4-methyl-3-(4-pyridin-3-yl-pyrimidin-2-ylamino)-phenyl]-benzamide (Imatinib, EGFR inhibitor).

Combinations comprising inhaled β2-adrenoreceptor agonist bronchodilators such as formoterol, albuterol or salmeterol with the IL-22 dimer are also suitable, but non-limiting, combinations useful for the treatment of respiratory viral infections.

Combinations of inhaled β2-adrenoreceptor agonist bronchodilators such as formoterol or salmeterol with ICS's are also used to treat both the bronchoconstriction and the inflammation (Symbicort® and Advair®, respectively). The combinations comprising these ICS and β2-adrenoreceptor agonist combinations along with the IL-22 dimer are also suitable, but non-limiting, combinations useful for the treatment of respiratory viral infections.

In some embodiments, the other therapeutic agent is an anticholinergic agent, which blocks the action of the neurotransmitter acetylcholine at synapses in the central and the peripheral nervous system. Therapeutic agents selectively block the binding of the neurotransmitter acetylcholine to its receptor in nerve cells, thus inhibiting parasympathetic nerve impulses, which are responsible for the involuntary movement of smooth muscles present in the gastrointestinal tract, urinary tract, lungs, and many other parts of the body. Anticholinergics are divided into three categories in accordance with their specific targets in the central and peripheral nervous system: antimuscarinic agents, ganglionic blockers, and neuromuscular blockers. Anticholinergic drugs are used to treat a variety of conditions including dizziness, extrapyramidal symptoms, gastrointestinal disorders (e.g., peptic ulcers, diarrhea, pylorospasm, diverticulitis, ulcerative colitis, nausea, and vomiting), genitourinary disorders (e.g., cystitis, urethritis, and prostatitis), insomnia, respiratory disorders (e.g., asthma, chronic bronchitis, and chronic obstructive pulmonary disease [COPD]), and sinus bradycardia due to a hypersensitive vagus nerve. Non-limiting examples of anticholinergic agents include atropine (Atropen), belladonna alkaloids, benztropine mesylate (Cogentin®), clidinium, cyclopentolate (Cyclogyl), darifenacin (Enablex), dicylomine, fesoterodine (Toviaz®), flavoxate (Urispas®), glycopyrrolate, homatropine hydrobromide, hyoscyamine (Levsinex), ipratropium (Atrovent®), orphenadrine, oxybutynin (Ditropan XL®), propantheline (Pro-banthine®), scopolamine, methscopolamine, solifenacin (VESIcare®), tiotropium (Spiriva®), tolterodine (Detrol®), trihexyphenidyl, and trospium.

In some embodiments, the other therapeutic agent is a mucolytic agent. Mucolytic agents can aid in the clearance of mucus from the upper and lower airways, including the lungs, bronchi, and trachea. Mucoactive drugs include expectorants, mucolytics, mucoregulators, and mucokinetics. These medications are used in the treatment of respiratory diseases that are complicated by the oversecretion or inspissation of mucus. Non-limiting examples of mucolytic agents include acetylcysteine (Mucomyst, Acys-5), ambroxol, bromhexine, carbocisteine, erdosteine, mecysteine, and dornase alfa.

In some embodiments, the other therapeutic agent is an antiviral agent. Most antivirals are used for specific viral infections, while a broad-spectrum antiviral is effective against a wide range of viruses. Unlike most antibiotics, antiviral drugs do not destroy their target pathogen; instead they inhibit their development. Antiviral drugs can include adamantane antivirals, antiviral boosters, antiviral combinations, antiviral interferons, chemokine receptor antagonist, integrase strand transfer inhibitor, miscellaneous antivirals, neuraminidase inhibitors, NNRTIs, NS5A inhibitors, nucleoside reverse transcriptase inhibitors (NRTIs), protease inhibitors, and purine nucleosides. Most currently available antiviral drugs are designed to help deal with HIV, herpes viruses, the hepatitis B and C viruses, and influenza A and B viruses.

Antiviral agents include, but are not limited to, valacyclovir, acyclovir, famciclovir, pritelivir, penciclovir, ganciclovir, valganciclovi, cidofovir, foscarnet, darunavir, glycyrrhizic acid, glutamine, FV-100, ASP2151, me-609, ASP2151, topical VDO, PEG-formulation (Devirex AG), vidarabine, cidofovir, crofelemer (SP-303T), EPB-348, CMXOO1, V212, NB-001, squaric acid, ionic zinc, sorivudine (ARYS-01), trifluridine, 882C87, merlin (ethanol and glycolic acid mixture), vitamin C, AIC316, versabase gel with Sarracenia purpurea, UB-621, lysine, edoxudine, brivudine, cytarabine, docosanol, tromantadine, resiquimod (R-848), imiquimod, resiquimod, tenofovir, tenofovir disoproxil fumarate, tenofovir alafenamide fumarate, include GSK208141 (gD2t, GSK glycoprotein D (gD)-Alum/3-deacylated form of monophosphoryl lipid A), Herpes Zoster GSK 1437173A, gD2-ASO4, Havrix™, gD-Alum, Zostavax/Zoster vaccine (V211, V212, V210), HSV529, HerpV (AG-707 rh-Hsc70 polyvalent peptide complex), VCL-HBO1, VCL-HMO1, pPJV7630, GEN-003, SPL7013 gel (VivaGel™), GSK324332A, GSK1492903A, VariZIG™, and Varivax, maraviroc, enfuvirtide, vicriviroc, cenicriviroc, lbalizumab, fostemsavir (BMS-663068), ibalizumab (TMB-355, TNX-355), PRO 140, b12 antibody, DCM205, DARPins, caprine antibody, bamlanivimab (LY-CoV555), VIR-576, enflivirtide (T-20), AMD11070, PR0542, SCH-C, T-1249, cyanovirin, griffithsen, lectins, pentafuside, dolutegravir, elvitegravir, raltegravir, globoidnan A, MK-2048, BI224436, cabotegravir, GSK 1265744, GSK-572, MK-0518, abacavir, didanosine, emtrictabine, lamivudine, stavudine, tenofovir, tenofovir disoporoxil fumarate, zidovudine, apricitabine, stampidine, elvucitabine, racivir, amdoxovir, stavudine, zalcitabine, festinavir, dideoxycytidine ddC, azidothymidine, tenofovir alafenamide fumarate, entecavir, delavirdine, efavirenz, etravirine (TMC-125), nevirapine, rilpivirine, doravirine, Calanolide A, capravirine, epivir, adefovir, dapivirine, lersivirine, alovudine, elvucitabine, TMC-278, DPC-083, amdoxovir, (−)-beta-D-2,6-diamino-purine dioxolane, MIV-210 (FLG), DFC (dexelvucitabine), dioxolane thymidine, L697639, atevirdine (U87201E), MIV-150, GSK-695634, GSK-678248, TMC-278, KP1461, KP-1212, lodenosine (FddA), 5-[(3,5-dichlorophenyl)thio]-4-isopropyl-1-(4-pyridylmethyl)imidazole-2-methanol carbamic acid, (−)-I2-D-2,6-diaminopurine dioxolane, AVX-754, BCH-13520, BMS-56190 ((4S)-6-chloro-4-[(1E)-cyclopropylethenyl]-3,-4-dihydro-4-trifiuoromethyl-2 (1H)-quinazolinone), TMC-120, L697639, atazanavir, darunavir, cobicistat, galidesivir, disulfiram, ASC09F (HIV protease inhibitor), nafamostat, gemcitabine hydrochloride, amodiaquine, mefloquine, loperamide, resveratrol, chloroquine, nitazoxanide, cyclosporine A, alisporivir, dasatinib, selumetinib, trametinib, rapamycin, saracatinib, chlorpromazine, triflupromazine, fluphenazine, thiethylperazine, promethazine, teicoplanin derivatives, mycophenolic acid, silvestrol, convalescent plasma, baloxavir marboxil, fosamprenavir, indinavir, nelfinavir, ritonavir, saquinavir, tipranavir, lopinavir, amprenavir, telinavir (SC-52151), droxinavir, emtriva, invirase, agenerase, TMC-126, mozenavir (DMP-450), JE-2147 (AG1776), L-756423, KNI-272, DPC-681, DPC-684, BMS 186318, droxinavir (SC-55389a), DMP-323, KNI-227, 1-[(2-hydroxyethoxy)methyl]-6-(phenylthio)-thymine, AG-1859, RO-033-4649, R-944, DMP-850, DMP-851, brecanavir (GW640385), nonoxynol-9, sodium dodecyl sulfate, Savvy (1.0% C31G), BufferGel®, carrageenans, VivaGel®, PRO-2000, also known as PRO 2000/5, naphthalene 2-sulfonate polymer, or polynaphthalene sulphonate, amphotericin B, sulfamethoxazole, trimethoprim, clarithromycin, daunorubicin, fluconazole, doxorubicin, anidulafungin, immune globulin, gamma globulin, dronabinol, megestrol acetate, atovaquone, rifabutin, pentamidine, trimetrexate glucuronate, leucovorin, alitretinoin gel, erythropoeetin, calcium hydroxylapatite, poly-L-lactic acid, somatropin rDNA, itraconazole, paclitaxel, voriconazole, cidofovir, fomivirsen, azithromycin, ruxolitinib, tocilizumab (Actemra®), sarilumab (Kevzara®), bevirimat, TRIM5 alpha, Tat antagonists, trichosanthin, abzyme, calanolide A, ceragenin, cyanovirin-N, diarylpyrimidines, epigallocatechin gallate (EGCG), foscarnet, griffithsin, hydroxycarbamide, miltefosine, portmanteau inhibitors, scytovirin, seliciclib, synergistic enhancers, tre recombinase, zinc finger protein transcription factor, KP-1461, BIT225, aplaviroc, atevirdine, brecanavir, capravirine, dexelvucitabine, emivirine, lersivirine, lodenosine, loviride, fomivirsen, glycyrrhizic acid (anti-inflammatory, inhibits 1 lbeta-hydroxysteroid dehydrogenase), zinc salts, cellulose sulfate, cyclodextrins, dextrin-2-sulfate, NCP7 inhibitors, AMD-3100, BMS-806, BMS-793, C31G, carrageenan, CD4-IgG2, cellulose acetate phthalate, mAb 2G12, mAb b12, Merck 167, plant lectins, poly naphthalene sulfate, poly sulfo-styrene, PRO2000, PSC-Rantes, SCH-C, SCH-D, T-20, TMC-125, UC-781, UK-427, UK-857, Carraguard (PC-515), brincidofovir (CMXOO1), zidovudine, virus-specific cytotoxic T cells, idoxuridine, podophyllotoxin, rifampicin, metisazone, interferon alfa 2b (Intron-A), peginterferon alfa-2a, ribavirin (Copegus, Rebetol®, Virazole), moroxydine, pleconaril, BCX4430, taribavirin (viramidine, ICN 3142), favipiravir (Avigan®), rintatolimod, ibacitabine, (5-iodo-2′-deoxycytidine), methisazone (metisazone), ampligen, Atripla®, combivir, imunovir, nexavir, trizivir, truvada, larnivudine, dideoxyadenosine, floxuridine, idozuridine, inosine pranobex, 2′-deoxy-5-(methylamino)uridine, digoxin, imiquimod, interferon type III, interferon type II, interferon type I, tea tree oil, glycyrrhizic acid, fialuridine, telbivudine, adefovir, etecavir, larnivudine, clevudine, asunaprevir, boceprevir, faldaprevir, grazoprevir, paritaprevir, lopinavir/ritonavir (Kaletra®), telaprevir, simeprevir, sofosbuvir, ACH-3102, daclatasvir, deleobuvir, elbasvir, ledipasvir, MK-3682, MK-8408, samatasvir, ombitasvir, entecavir, elderberry sambucus, umifenovir, amantadine, rimantadine, oseltamivir, zanamivir, peramivir, laninamivir, pyrrole polyamides, or salts, solvates, and/or combinations thereof.

In some embodiments, the antiviral agent is selected from the group consisting of remdesivir, lopinavir/ritonavir (Kaletra®), IFNs (e.g., IFN-α such as IFN-α2a or IFN-α2b, IFN-3, IFN-γ), lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir (Tamiflu®), zanamivir, peramivir, amantadine, rimantadine, favipiravir (Avigan®), laninamivir, ribavirin (Copegus, Rebetol®, Virazole), umifenovir (Arbidol®), and any combinations thereof.

In some embodiments, any of the therapeutic agents described in Li and Clercq (“Therapeutic options for the 2019 novel coronavirus (2019-nCoV)”, Nature Reviews Drug Discovery, Feb. 10, 2020; including Supplementary Table 1) can be used as another therapeutic agent described herein in combination with IL-22 dimer, for treating organ injury or failure associated with any viral infection, such as infection by SARS-CoV (e.g., SARS), MERS-CoV (e.g., MERS), SARS-CoV-2 (e.g., COVID-19), H1N1 (e.g., H1N1 swine flu), or H5N1 (e.g., H5N1 bird flu). The content of which is incorporated herein by reference in its entirety.

In some embodiments, when treating virus-induced organ injury or failure associated with SARS-CoV-2 infection, the other therapeutic agent is selected from the group consisting of remdesivir (Veklury®), dexamethasone, hydrocortisone, methylprednisolone, convalescent plasma, bamlanivimab (LY-CoV555), LY-CoV016, casirivimab and imdevimab (REGN-COV2), AZD7442, VIR-7831, BRII-196, BRII-198, lopinavir/ritonavir (Kaletra®, e.g., tablet), IFN-α (e.g., IFN-α2a or IFN-α2b, via inhalation), favipiravir, lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, and any combinations thereof. In some embodiments, when treating virus-induced organ injury or failure associated with SARS-CoV-2 infection, the other therapeutic agent is lopinavir/ritonavir (Kaletra®) and IFN-α (e.g., IFN-α2a or IFN-α2b, via inhalation). In some embodiments, when treating virus-induced organ injury or failure associated with SARS-CoV-2 infection, the other therapeutic agent is remdesivir (Veklury®).

In some embodiments, when treating virus-induced organ injury or failure associated with H1N1 or H5N1 infection, the other therapeutic agent is selected from the group consisting of oseltamivir, zanamivir, peramivir, favipiravir, umifenovir (Arbidol®), teicoplanin derivatives, benzo-heterocyclic amine derivative, pyrimidine, baloxavir marboxil, lopinavir/ritonavir (Kaletra®, e.g., tablet), INF-α (e.g., IFN-α2a, IFN-α2b, via inhalation), and any combinations thereof. In some embodiments, when treating virus-induced organ injury or failure associated with H1N1 or H5N1 infection, the other therapeutic agent is lopinavir/ritonavir (Kaletra®) and INF-α (e.g., IFN-α2a or IFN-α2b, via inhalation). In some embodiments, when treating virus-induced organ injury or failure associated with H1N1 or H5N1 infection, the other therapeutic agent is oseltamivir.

Remdesivir (GS-5734 or Veklury®) is an antiviral drug, a novel nucleotide analog prodrug (phosphoramidate prodrug of an adenine derivative), developed by Gilead Sciences as a treatment for Ebola virus disease (Phase 1, NCT03719586) and Marburg virus infections. Its reported mechanism of action is targeting RNA dependent RNA polymerase (RdRp) and terminating the non-obligate chain. It has also shown antiviral activity against more distantly related single stranded RNA viruses such as respiratory syncytial virus, Junin virus, Lassa fever virus, Nipah virus, Hendra virus, and coronaviruses (including MERS and SARS viruses). Recently, remdesivir demonstrated some fairly good antiviral activity against SARS-CoV-2 in a small number of Chinese patients. Remdesivir was previously under Phase 3 for treating COVID-19 (NCT04252664, NCT04257656), and now is the first and only antiviral approved by FDA for the treatment of patients requiring hospitalization for COVID-19.

Favipiravir (T-705 or Avigan®) is a guanine analogue approved for treating influenza in Japan. It can effectively inhibit RdRp of RNA viruses such as influenza, Ebola, yellow fever, chikungunya, norovirus, and enterovirus. It is currently under randomized trials for treating COVID-19 in combination with baloxavir marboxil (ChiCTR2000029544) or in combination with IFN-α (ChiCTR2000029600).

Ribavirin is a guanine derivative approved for treating HCV and RSV infection. Its drug target is RdRp, and its reported mechanism is to inhibit viral RNA synthesis and mRNA capping. Ribavirin is currently under a randomized clinical trial for treating COVID-19 in combination with a pegylated interferon (ChiCTR2000029387), and a randomized clinical trial for SARS (NCT00578825). Ribavirin is expected to treat SARS, MERS, and COVID-19.

Galidesivir (BCX4430) is an adenosine analogue that targets RdRp. Its reported mechanism is to inhibit viral RNA polymerase function by terminating nonobligate RNA chain. Galidesivir is currently under Phase 1 for treating Marburg virus (NCT03800173), and Phase I for treating yellow fever (NCT03891420). Galidesivir is expected to be a broad-spectrum antiviral agent (e.g. SARS-CoV, MERS-CoV, IAV).

Disulfiram is a protease inhibitor approved for chronic alcohol dependence. It has been reported to inhibit papain-like protease (PLpro) of MERS-CoV and SARS-CoV in cell experiments.

Lopinavir is a protease inhibitor approved for treating HIV infection. It is currently under Phase 3 trial for treating COVID-19 (NCT04252274, NCT04251871, NCT04255017, ChiCTR2000029539), and Phase 2/3 trial for MERS (NCT02845843). Its reported mechanism of action is to inhibit 3CLpro. It is expected to treat infections by MERS-CoV, SARS-CoV, SARS-CoV-2, HCoV-229E, and HPV.

Ritonavir is a protease inhibitor approved for treating HIV infection. It is currently under Phase 3 trial for treating COVID-19 (NCT04251871, NCT04255017, NCT04261270), and Phase 2/3 trial for MERS (NCT02845843). Its reported mechanism of action is to inhibit 3CLpro. It is expected to treat infections by MERS-CoV and SARS-CoV-2.

Lopinavir/ritonavir (LPV/r; Kaletra®) is a fixed dose combination medication for treating and preventing HIV/AIDS. It combines lopinavir with a low dose of ritonavir. Common side effects include diarrhea, vomiting, feeling tired, headaches, and muscle pains. Severe side effects may include pancreatitis, liver problems, and high blood sugar. Administration route can involve tablet, capsule, or solution taken by mouth.

Griffithsin a red-alga-derived lectin, and is currently under Phase 1 trial for the prevention of HIV transmission (NCT02875119 and NCT04032717). Its reported mechanism of action is binding to the SARS-CoV spike glycoprotein and inhibiting viral entry. It is expected to treat SARS-CoV infection.

Interferons (IFNs) are a group of signaling molecules produced by host cells in response to viral infection. IFNs belong to cytokines. IFNs can protect cells from virus infections, activate immune cells (e.g., NK cells, macrophages), increase host defenses by up-regulating antigen presentation (by increasing the expression of major histocompatibility complex (MHC) antigens). There are three classes of IFNs: Type I IFN, Type II IFN, and Type III IFN. Some IFNs have been approved for metastatic renal cell carcinoma (IFN-α2a), melanoma (IFN-α2b), multiple sclerosis (IFNβ1a, IFNβ1b), and chronic granulomatous disease (IFN-γ). IFNα belongs to Type I IFN. It is mainly produced by plasmacytoid dendritic cells (pDCs), and involved in innate immunity against viral infection. It is expected to treat SARS-CoV, MERS-CoV, or SARS-CoV-2 infection, by stimulating innate antiviral responses in infected patients.

Oseltamivir (Tamiflu®) is an antiviral agent used to treat and prevent influenza A and influenza B (flu). Some H1N1 and H5N1 patients were found to be resistant to oseltamivir treatment. Zanamivir (Relenza®) is an antiviral agent (neuraminidase inhibitor) used to treat and prevent influenza A and influenza B (flu). It was used to treat H1N1 in 2009. Peramivir (Rapivab®) is an antiviral agent (neuraminidase inhibitor) used to treat and prevent influenza. Some H1N1 patients had highly reduced peramivir inhibition due to H275Y NA mutation.

Chloroquine is an approved immune modulator for treating malaria and certain amoeba infections. It is reported to be a lysosomatropic base that appears to disrupt intracellular trafficking and viral fusion events. It is currently under an open-label trial for COVID-19 (ChiCTR2000029609). It is expected to treat SARS-CoV, MERS-CoV, or SARS-CoV-2 infection. Nitazoxanide has been approved for diarrhea treatment. Its reported mechanism of action is to induce the host innate immune response to produce interferons. It is expected to be a broad-spectrum antiviral agent (e.g., coronaviruses such as SARS-CoV-2).

In some embodiments, the other therapeutic agent is an anti-fibrotic agent. In some embodiments, the anti-fibrotic agent is selected from the group consisting of nintedanib, pirfenidone, and N-Acetylcysteine (NAC).

In some embodiments, the other therapeutic agent is an antibody, such as an antibody that bind viruses and help destroy them. In some embodiments, the antibody is selected from the group consisting of bamlanivimab (LY-CoV555), LY-CoV016, casirivimab and imdevimab (REGN-COV2), AZD7442, VIR-7831, BRII-196, BRII-198, and any combinations thereof. Bamlanivimab was designed to block SARS-CoV-2 from entering and infecting human cells. On Nov. 9, 2020, the FDA issued an EUA for bamlanivimab to treat mild or moderate COVID-19 in patients 12 years and older who are at high risk of hospitalization. REGN-COV2 is an antibody cocktail made of casirivimab and imdevimab. On Nov. 21, 2020, the FDA issued an EUA for casirivimab and imdevimab to be used together to treat mild or moderate COVID-19 in patients 12 years and older who are at high risk of hospitalization. More data are being gathered.

In some embodiments, the other therapeutic agent is a vaccine. In some embodiments, In some embodiments, the vaccine is a COVID-19 vaccine. In some embodiments, the vaccine is selected from the group consisting of RNA vaccines such as tozinameran (Comirnaty®; the Pfizer-BioNTech vaccine) and mRNA-1273 (CX-024414; the Moderna vaccine); conventional inactivated vaccines such as BBIBP-CorV (from Sinopharm), BBV152 (from Bharat Biotech), CoronaVac (from Sinovac), and WIBP (from Sinopharm); viral vector vaccines such as Sputnik V (from the Gamaleya Research Institute), AZD1222 (the Oxford-AstraZeneca vaccine), and Ad5-nCoV (from CanSino Biologics); and peptide vaccine such as EpiVacCorona (from the Vector Institute).

In some embodiments, the second therapy can comprise any of current treatments for specific organ dysfunction, such as dysfunction or failure in heart, kidney, liver, lung, etc. In some embodiments, the second therapy can comprise any of current treatments for respiratory failure, including, but are not limited to, increasing the patient's oxygen levels using an oxygen mask, mechanical oxygenation using a ventilator or, in the most severe case, extracorporeal membrane oxygenation (ECMO) which involves circulating the patient's blood outside the body and adding oxygen to it artificially. In some embodiments, the second therapy can comprise any of current treatments for congestive heart failure, including, but are not limited to, cardiac resynchronization therapy (CRT) or biventricular pacing, ventricular assist devices (VADs), and cardioverter-defibrillators. In some embodiments, the second therapy can comprise any of current treatments for kidney failure, such as dialysis.

It is possible to combine any IL-22 dimer of the invention with one or more additional active therapeutic agents in a unitary dosage form for simultaneous or sequential administration to a patient. The combination therapy may be administered as a simultaneous or sequential regimen. When administered sequentially, the combination may be administered in two or more administrations.

Co-administration of an IL-22 dimer described herein with one or more other active therapeutic agents (or second therapy) generally refers to simultaneous or sequential administration of an IL-22 dimer and one or more other active therapeutic agents (or second therapy), such that therapeutically effective amounts of the IL-22 dimer and one or more other active therapeutic agents (or the effectiveness of second therapy) are both present in the body of the patient.

Co-administration includes administration of unit dosages of the IL-22 dimer described herein before or after administration of unit dosages of one or more other active therapeutic agents (or a second therapy), for example, administration of the IL-22 dimer within seconds, minutes, or hours of the administration of one or more other active therapeutic agents (or a second therapy). For example, a unit dose of an IL-22 dimer can be administered first, followed within seconds or minutes by administration of a unit dose of one or more other active therapeutic agents (or a second therapy). Alternatively, a unit dose of one or more other therapeutic agents (or a second therapy) can be administered first, followed by administration of a unit dose of an IL-22 dimer within seconds or minutes. In some cases, it may be desirable to administer a unit dose of an IL-22 dimer of the invention first, followed, after a period of hours (e.g., 1-12 hours), by administration of a unit dose of one or more other active therapeutic agents. In other cases, it may be desirable to administer a unit dose of one or more other active therapeutic agents (or a second therapy) first, followed, after a period of hours (e.g., 1-12 hours), by administration of a unit dose of an IL-22 dimer of the invention. In some embodiments, the IL-22 dimer is administered prior to, or subsequent to, the administration of the other therapeutic agent or second therapy, such as about any of 5 min, 10 min, 30 min, 1 hr, 2 hr, 3 hr, 4 hr, 5 hr, 6 hr, 7 hr, 8 hr, 9 hr, 10 hr, 11 hr, 12 hr, 13 hr, 14 hr, 15 hr, 16 hr, 17 hr, 18 hr, 19 hr, 20 hr, 21 hr, 22 hr, 23 hr, 24 hr, 2 days, 3 days, 4 days, 5 days, 6 days, a week, or longer, prior to, or subsequent to, the administration of the other therapeutic agent or second therapy.

In some embodiments, the IL-22 dimer is administered simultaneously with the other therapeutic agent or a second therapy. In some embodiments, the IL-22 dimer is administered subsequent to the other therapeutic agent or a second therapy. In some embodiments, the IL-22 dimer is administered prior to the other therapeutic agent or a second therapy.

The combination therapy may provide “synergy” and “synergistic”, i.e. the effect achieved when the active ingredients used together is greater than the sum of the effects that results from using the agents (or therapy) separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the agents (or therapy) are administered or delivered sequentially, e.g. in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together. A synergistic anti-viral effect denotes an antiviral effect which is greater than the predicted purely additive effects of the individual agents of the combination.

### III. Methods of Preparation

The IL-22 dimer described herein may be prepared by any of the known protein expression and purification methods in the art, such as recombinant DNA technology. DNA sequence encoding the IL-22 dimer can be fully synthesized. After obtaining such sequence, it is cloned into a suitable expression vector, then transfected into a suitable host cell. The transfected host cells are cultured, and the supernatant is harvested and purified to obtain the IL-22 dimer of the present invention.

In some embodiments, the isolated nucleic acid encoding IL-22 monomeric subunit or IL-22 dimer (e.g., FIG. 1) is inserted into a vector, such as an expression vector, a viral vector, or a cloning vector, at restriction sites using known techniques. In some embodiments, a single nucleotide sequence encoding IL-22 monomeric subunit (or IL-22 dimer) is inserted into a cloning or expression vector. In some embodiments, a nucleotide sequence encoding the IL-22 monomer and a nucleotide sequence encoding a carrier protein may be separately inserted into a cloning or expression vector in such a manner that when the nucleotide sequence is expressed as a protein, a continuous polypeptide is formed. In some embodiments, a nucleotide sequence encoding a linker, a nucleotide sequence encoding a dimerization domain, and a nucleotide sequence encoding an IL-22 monomer may be separately inserted into a cloning or expression vector in such a manner that when the nucleotide sequence is expressed as a protein, a continuous polypeptide is formed. In some embodiments, the nucleotide sequence encoding IL-22 monomeric subunit (or IL-22 dimer) may be fused to a nucleotide sequence encoding an affinity or identification tag, including, but not limited to, a His-tag, FLAG-tag, SUMO-tag, GST-tag, antibody-tag, or MBP-tag. Signal sequences may be selected to allow the expressed polypeptide to be transported outside of the host cell. In some embodiments, the isolated nucleic acids further comprise a nucleic acid sequence encoding a signal peptide to be expressed at the N-terminus of the polypeptide.

For expression of the nucleic acids, the vector may be introduced into a host cell (e.g., eukaryotic or prokaryotic cells) using known techniques to allow expression of the nucleic acids within the host cell. In some embodiments, IL-22 dimer or IL-22 monomeric subunits may be expressed in vitro. The expression vectors may contain a variety of elements for controlling expression, including without limitation, promoter sequences, transcription initiation sequences, enhancer sequences, selectable markers, and signal sequences. These elements may be selected as appropriate by a person of ordinary skill in the art. For example, the promoter sequences may be selected to promote the transcription of the polynucleotide in the vector. Suitable promoter sequences include, without limitation, T7 promoter, T3 promoter, SP6 promoter, beta-actin promoter. EFla promoter, CMV promoter, and SV40 promoter. Enhancer sequences may be selected to enhance the transcription of the nucleic acids. Selectable markers may be selected to allow selection of the host cells inserted with the vector from those not, for example, the selectable markers may be genes that confer antibiotic resistance.

The host cells containing the vector may be useful in expression or cloning of the isolated nucleic acids. The expression host cell may be any cell able to express IL-22 dimers. Suitable host cells can include, without limitation, prokaryotic cells, fungal cells, yeast cells, or higher eukaryotic cells such as mammalian cells. Suitable prokaryotic expression host cells may include, but are not limited to, Escherichia coli, Erwinia, Klebsiella, Proteus, Salmonella, Serratia, Shigella, Bacillus subtilis, Bacillus lichenmformis, Pseudomonas, and Streptomyces. Eukaryotic cell, such as fungi or yeast, may also be suitable for expression of IL-22 monomeric subunits, for example, but not limited to, Saccharomyces, Schizosaccharomyces pombe, Kluyveromyces lactis, Kluyveromyces fragilis, Kluyveromyces waltii, Kluyveromyces drosophilarum, Kluyveromyces thermotolerans, Kluyveromyces marxianus, Pichia pastoris, Neurospora crassa, Schwanniomyces, Penicillium, Tolypocladium, Synechococcus and Aspergillus. Plant or algal cells may also be suitable for expression of IL-22 monomeric subunits, such as Chlamydomonas. Eukaryotic cell derived from multicellular organisms may also be suitable for expression of IL-22 monomeric subunits, for example, but not limited to, invertebrate cells such as Drosophila S2 and Spodoptera Sf9, or mammalian cells such as Chinese Hamster Ovary (CHO) cells, COS cells, human embryonic kidney cells (such as HEK293 cells), murine testis trophoblastic cells, human lung cells, and murine breast cancer cells. Higher eukaryotic cells, in particular, those derived from multicellular organisms can be used for expression of glycosylated polypeptides. Suitable higher eukaryotic cells include, without limitation, invertebrate cells and insect cells, and vertebrate cells. In some embodiments, the host cell used to express IL-22 monomeric subunit or IL-22 dimer is Chinese Hamster Ovary (CHO) cell.

The vector can be introduced to the host cell using any suitable methods known in the art, including, but not limited to, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transfection and transformation of cells for expression of a vector of interest are well known in the art. In some embodiments, the host cells comprise a first vector encoding a first polypeptide (e.g. a first IL-22 monomeric subunit) and a second vector encoding a second polypeptide (e.g. a second IL-22 monomeric subunit). In some embodiments, the host cells comprise a single vector comprising isolated nucleic acids encoding a first polypeptide (e.g. a first IL-22 monomeric subunit) and a second polypeptide (e.g. a second IL-22 monomeric subunit).

After the IL-22 monomeric subunit (or IL-22 dimer) cloning plasmid is transformed or transfected into a host cell, the host cells containing the vector is cultured and IL-22 monomeric subunit (or IL-22 dimer) is recovered from the cell culture. The isolated host cells are cultured under conditions that allow expression of the isolated nucleic acids inserted in the vectors. Suitable conditions for expression of polynucleotides may include, without limitation, suitable medium, suitable density of host cells in the culture medium, presence of necessary nutrients, presence of supplemental factors, suitable temperatures and humidity, and absence of microorganism contaminants. In some embodiments, can be grown on conventional nutrient media and protein expression induced, if necessary. In some embodiments, the expression of IL-22 monomeric subunits (or IL-22 dimer) do not require inducement. A person with ordinary skill in the art can select the suitable conditions as appropriate for the purpose of the expression.

In some embodiments, the polypeptides (e.g. IL-22 monomeric subunit) expressed in the host cell can form a dimer and thus produce an IL-22 dimer described herein. In some embodiments, the polypeptides expressed in the host cell can form a polypeptide complex which is a homodimer. In some embodiments, the host cells express a first polypeptide (e.g. a first IL-22 monomeric subunit) and a second polypeptide (e.g. a second IL-22 monomeric subunit), the first polypeptide and the second polypeptide can form a polypeptide complex which is a heterodimer (e.g., heterodimeric IL-22 dimer). In some embodiments, IL-22 monomeric subunits will require further inducement, such as by supplying an oxidation compound (such as hydrogen peroxide or a catalytic metal), UV light, or a chemical crosslinker (such as formaldehyde, 1,6-bismaleimidohexane, 1,3-dibromo-2-propanol, bis(2-chloroethyl)sulfide, or glutaraldehyde). In some embodiments, the forming of IL-22 dimers do not require inducement.

In some embodiments, the IL-22 dimer may be formed inside the host cell. For example, the dimer may be formed inside the host cell with the aid of relevant enzymes and/or cofactors. In some embodiments, the IL-22 dimer may be secreted out of the cell. In some embodiments, a first IL-22 monomeric subunit and a second IL-22 monomeric subunit may be secreted out of the host cell and form an IL-22 dimer outside of the host cell.

In some embodiments, a first IL-22 monomeric subunit and a second IL-22 monomeric subunit may be separately expressed and allowed to dimerize to form the IL-22 dimer under suitable conditions. For example, the first IL-22 monomeric subunit and the second IL-22 monomeric subunit may be combined in a suitable buffer and allow the first IL-22 monomeric subunit and the second IL-22 monomeric subunit to dimerize through appropriate interactions such as hydrophobic interactions. In some embodiments, the first IL-22 monomeric subunit and the second IL-22 monomeric subunit may be combined in a suitable buffer containing an enzyme and/or a cofactor which can promote the dimerization of the first IL-22 monomeric subunit and the second IL-22 monomeric subunit. In some embodiments, the first IL-22 monomeric subunit and the second IL-22 monomeric subunit may be combined in a suitable vehicle and allow them to react with each other in the presence of a suitable reagent and/or catalyst.

The expressed IL-22 monomeric subunit and/or the IL-22 dimer can be collected using any suitable methods. The IL-22 monomeric subunit and/or the IL-22 dimer can be expressed intracellularly, in the periplasmic space or be secreted outside of the cell into the medium. If the IL-22 monomeric subunit and/or the IL-22 dimer are expressed intracellularly, the host cells containing the IL-22 monomeric subunit and/or the IL-22 dimer may be lysed and IL-22 monomeric subunit and/or the IL-22 dimer may be isolated from the lysate by removing the unwanted debris by centrifugation or ultrafiltration. If the IL-22 monomeric subunit and/or the IL-22 dimer is secreted into periplasmic space of E. coli, the cell paste may be thawed in the presence of agents such as sodium acetate (pH 3.5), EDTA, and phenylmethylsulfonylfluoride (PMSF) for about 30 min, and cell debris can be removed by centrifugation (Carter et al., BioTechnology 10:163-167 (1992)). If the IL-22 monomeric subunit and/or the IL-22 dimer is secreted into the medium, the supernatant of the cell culture may be collected and concentrated using a commercially available protein concentration filter, for example, an Amincon or Millipore Pellicon ultrafiltration unit. A protease inhibitor and/or an antibiotic may be included in the collection and concentration steps to inhibit protein degradation and/or growth of contaminated microorganisms.

The expressed IL-22 monomeric subunit(s) and/or the IL-22 dimer can be further purified by a suitable method, such as without limitation, affinity chromatography, hydroxylapatite chromatography, size exclusion chromatography, gel electrophoresis, dialysis, ion exchange fractionation on an ion-exchange column, ethanol precipitation, reverse phase HPLC, chromatography on silica, chromatography on heparin sepharose, chromatography on an anion or cation exchange resin (such as a polyaspartic acid column), chromatofocusing, SDS-PAGE, and ammonium sulfate precipitation (see, for review, Bonner, P. L., Protein purification, published by Taylor & Francis. 2007; Janson, J. C., et al, Protein purification: principles, high resolution methods and applications, published by Wiley-VCH, 1998). In some embodiments, IL-22 monomeric subunit(s) and/or IL-22 dimer may be purified using affinity chromatography, ion exchange chromatography, viral inactivation, viral filtration, mixed-mode chromatography, reverse-phase HPLC, size-exclusion chromatography, tangential flow filtration, precipitation, or ultracentrifugation. In some embodiments, an affinity tag fused to purify the IL-22 monomeric subunit and/or IL-22 dimer may be removed.

In some embodiments, the IL-22 monomeric subunit(s) and/or IL-22 dimer can be purified by affinity chromatography. In some embodiments, protein A chromatography or protein A/G (fusion protein of protein A and protein G) chromatography can be useful for purification of IL-22 monomeric subunit(s) and/or IL-22 dimer comprising a component derived from antibody CH2 domain and/or CH3 domain (Lindmark et al., J. Immunol. Meth. 62:1-13 (1983)); Zettlit, K. A., Antibody Engineering, Part V, 531-535, 2010). In some embodiments, protein G chromatography can be useful for purification of IL-22 monomeric subunit(s) and/or IL-22 dimer comprising IgG γ3 heavy chain (Guss et al., EMBO J. 5:1567 1575 (1986)). In some embodiments, protein L chromatography can be useful for purification of IL-22 monomeric subunit(s) and/or IL-22 dimer comprising κ light chain (Sudhir, P., Antigen engineering protocols, Chapter 26, published by Humana Press, 1995; Nilson, B. H. K. at al, J. Biol. Chem., 267, 2234-2239 (1992)). The matrix to which the affinity ligand is attached is most often agarose, but other matrices are available. Mechanically stable matrices such as controlled pore glass or poly(styrenedivinyl) benzene allow for faster flow rates and shorter processing times than can be achieved with agarose. Where the IL-22 monomeric subunit or IL-22 dimer comprises an additional CH3 domain, the Bakerbond ABX resin (J. T. Baker, Phillipsburg, N.J.) is useful for purification.

The exemplary preparation methods of IL-22 dimers can be referred to Patent Application PCT/CN2011/079124 filed by Generon (Shanghai) Corporation, Ltd. (now Evive Biotechnology (Shanghai) Ltd) on Aug. 30, 2011, incorporated herein by reference in its entirety.

### EXAMPLES

The examples below are intended to be purely exemplary of the invention and should therefore not be considered to limit the invention in any way. The following examples and detailed description are offered by way of illustration and not by way of limitation. For the embodiments in which details of the experimental methods are not described, such methods are carried out according to conventional conditions such as those described in Sambrook et al. Molecular Cloning: A Laboratory Manual (New York: Cold Spring Harbor Laboratory Press, 1989), or as suggested by the manufacturers.

### Example 1. Study of Therapeutic Effects of Recombinant IL-22 Dimer (F-652) in Combination with Antiviral Agent on Mouse Model of H1N1 Infection

### Methods

F-652 is recombinant IL-22 dimer consisting of two monomeric subunits each comprising a sequence shown in SEQ ID NO: 24.

Female BALB/c mice (5-6 weeks of age, weight range 15-18 g) were randomized into three groups (14 mice each), designated as Model control group, Oseltamivir treatment group, and (F-652+oseltamivir) treatment group.

All animals were challenged with Influenza A virus subtype H1N1 (“H1N1”; strain A/California/07/2009) nasal drops on Day 0 at a dose of 1×LD₅₀, i.e., 10⁴TCID₅₀ per mouse. Test drugs or placebo were administered starting from 2 hours after viral challenge. For the Oseltamivir treatment group, animals were intragastrically administered with oseltamivir (Tamiflu®, Roche) at a dose of 30 mg/kg once daily for 5 consecutive days. For the (F-652+oseltamivir) treatment group, animals were intragastrically administered with Oseltamivir (Tamiflu®, Roche) at a dose of 30 mg/kg once daily for 5 consecutive days, and intravenously injected with F-652 (in PBS solution containing 0.05% Tween 80) at a dose of 30 μg/kg every two days for 6 doses total. The Model control group was intravenously injected with equal volume of vehicle.

Animal survival rate and clinical manifestations were monitored and recorded daily. On Day 5, six mice from each group were selected and euthanized, lung tissues were collected. Of which, three lung tissues were fixed, and hematoxylin and eosin (H&E) stain was performed. Changes on lung cells were observed and pathological scores were obtained. The other three lung tissues were examined for viral titers. At the end of the study (Day 14), all mice were euthanized. Lung tissues were collected, fixed, and H&E stain was performed. Changes on lung cells were observed and pathological scores were obtained.

### Results

At the end of the study, the survival rate of mice in the Model control group was 50% (4/8), and the survival rate of mice in the Oseltamivir treatment group was 62.5% (5/8). The survival rate of mice in the (F-652+oseltamivir) treatment group was 75% (6/8), higher than those of the Oseltamivir treatment group and the Model control group. See FIG. 4.

On Day 5 after viral challenge, the average virus titer in the Model control group was log₁₀^{3.61}TCID₅₀, the average virus titer in the Oseltamivir treatment group was log₁₀^{2.50}TCID₅₀, and the average virus titer in the (F-652+oseltamivir) treatment group was log₁₀^{2.56}TCID₅₀. The average virus titers in the Oseltamivir treatment group and the (F-652+oseltamivir) treatment group were both lower than that of the Model control group.

On Day 5 after drug administration, the average of total pathological score of the Oseltamivir treatment group showed certain decrease compared to that of the Model control group. The average of total pathological score of the (F-652+oseltamivir) treatment group was 9.00±2.00, lower than that of the Oseltamivir treatment group (10.67±3.51). See Table 1.

TABLE 1
Pulmonary histopathological scores on Day 5 after viral challenge

| | | | Exudation of | Alveolar | | | | | |
|---|---|---|---|---|---|---|---|---|---|
| | | | inflammatory | septum and | Bronchial | | | | |
| | | | cells, serous, | perivascular | epithelial | | | | |
| | | Alveolar | and cellulose | infiltration of | cell | | | | |
| | Animal | septum | in the alveolar | inflammatory | degeneration | Vasodilatation | | Total | |
| Group | # | widening | cavity | cells | and necrosis | congestion | Haemorrhage | score | Mean ± SD |
| Oseltamivir | G2-1 | 2 | 0 | 2 | 2 | 1 | 0 | 7 | 10.67 ± 3.51 |
| treatment | G2-2 | 3 | 2 | 3 | 3 | 2 | 1 | 14 | |
| group | G2-3 | 2 | 2 | 2 | 2 | 2 | 0 | 11 | |
| (F-652 + | G3-1 | 2 | 1 | 2 | 3 | 1 | 0 | 9 | 9.00 ± 2.00 |
| oseltamivir) | G3-2 | 1 | 0 | 2 | 2 | 2 | 0 | 7 | |
| treatment | G3-3 | 2 | 2 | 2 | 3 | 2 | 0 | 11 | |
| group | G6-2 | 3 | 2 | 3 | 3 | 2 | 0 | 13 | |
| | G6-3 | 3 | 2 | 3 | 3 | 2 | 0 | 13 | |
| Model | G1-1 | 4 | 3 | 3 | 3 | 3 | 0 | 16 | 15.67 ± 0.58 |
| control | G1-2 | 3 | 3 | 3 | 3 | 3 | 0 | 15 | |
| group | G1-3 | 3 | 3 | 3 | 3 | 3 | 1 | 16 | |

At the end of the study (Day 14), the average of total pathological score of the Oseltamivir treatment group showed certain decrease compared to that of the Model control group. The average of total pathological score of the (F-652+oseltamivir) treatment group was 14.67±1.63, lower than that of the Oseltamivir treatment group (15.40±1.95). See Table 2.

TABLE 2
Pulmonary histopathological scores on Day 14 after viral challenge

| | | | Exudation of | Alveolar | | | | | |
|---|---|---|---|---|---|---|---|---|---|
| | | | inflammatory | septum and | | | | | |
| | | | cells, serous, | perivascular | Bronchial | | | | |
| | | Alveolar | and cellulose | infiltration of | epithelial | | | | |
| | Animal | septum | in the alveolar | inflammatory | cell | Vasodilatation | | Total | |
| Group | # | widening | cavity | cells | hyperplasia | congestion | Haemorrhage | score | Mean ± SD |
| Oseltamivir | G2-1 | 3 | 2 | 3 | 2 | 4 | 0 | 14 | 15.40 ± 1.95 |
| treatment | G2-2 | 2 | 2 | 2 | 2 | 4 | 1 | 13 | |
| group | G2-3 | 4 | 3 | 4 | 2 | 4 | 1 | 18 | |
| | G2-4 | 3 | 2 | 3 | 2 | 4 | 2 | 16 | |
| | G2-5 | 3 | 3 | 3 | 2 | 4 | 1 | 16 | |
| (F-652 + | G3-1 | 2 | 2 | 2 | 1 | 4 | 1 | 12 | 14.67 ± 1.63 |
| oseltamivir) | G3-2 | 3 | 2 | 3 | 2 | 4 | 1 | 15 | |
| treatment | G3-3 | 3 | 3 | 3 | 2 | 3 | 1 | 15 | |
| group | G3-4 | 3 | 3 | 3 | 2 | 3 | 0 | 14 | |
| | G3-5 | 4 | 3 | 3 | 2 | 4 | 1 | 17 | |
| | G3-6 | 3 | 2 | 3 | 2 | 4 | 1 | 15 | |
| Model | G1-1 | 3 | 4 | 4 | 2 | 4 | 2 | 19 | 17.67 ± 1.15 |
| control | G1-2 | 3 | 4 | 4 | 1 | 4 | 1 | 17 | |
| group | G1-3 | 3 | 3 | 4 | 1 | 4 | 2 | 17 | |

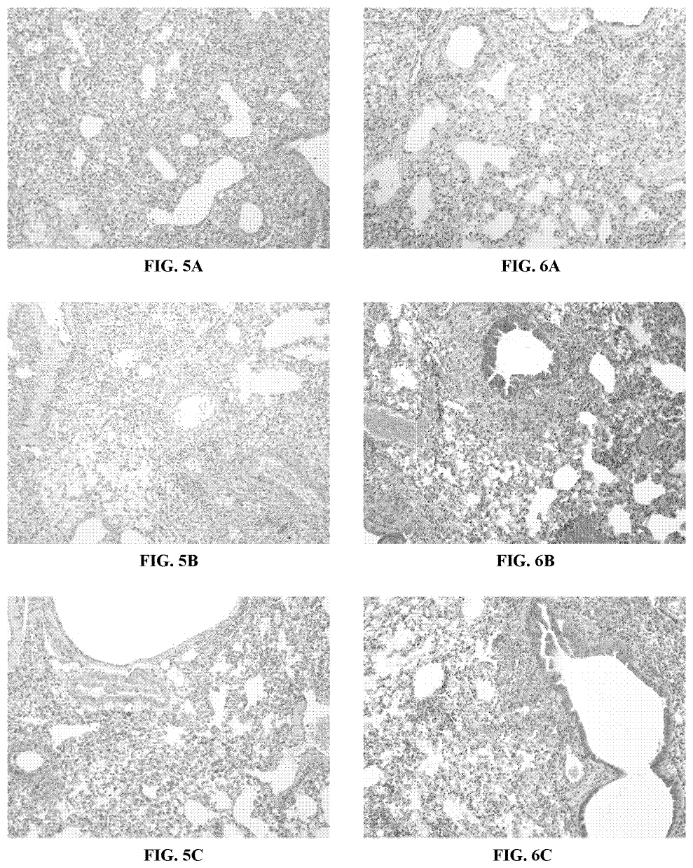

Histopathological evaluations and morphological changes of lung tissues on Day 5 (FIGS. 5A-5C) and Day 14 (FIGS. 6A-6C) showed that lung injury was reduced in the Oseltamivir treatment group (FIGS. 5B and 6B) in comparison to that of the model control group (FIGS. 5A and 6A). The extent of lung injury was further reduced in the (F-652+oseltamivir) treatment group (FIGS. 5C and 6C).

These results showed that oseltamivir treatment alone was able to reduce death rate, viral titers, and lung pathological injury in mice model of Influenza virus (e.g., H1N1) infection. Further intravenous administration of F-652 in combination with oseltamivir treatment could further reduce mortality and ameliorate lung injury in mice model of Influenza virus (e.g., H1N1) infection, compared to oseltamivir single therapy. Hence, the results demonstrated that combination therapy of oseltamivir and F-652 could reduce mortality and lung injury induced by Influenza virus (e.g., H1N1) infection, and promote lung tissue repair.

### Example 2. Randomized Controlled Study of Recombinant IL-22 Dimer (F-652) in Treating Severe COVID-19 (e.g., Severe Pneumonia) Due to SARS-CoV-2 Infection, in Combination with Conventional Antiviral Regimen

### Study Description

This is a randomized controlled study to investigate the safety and efficacy of F-652 (recombinant human IL-22 IgG2-Fc) in combination with conventional antiviral regimen in patients who have severe COVID-19 (e.g., severe pneumonia) due to SARS-CoV-2 infection. Effect of F-652 on liver, kidney and other organ functions in patients with severe pneumonia are evaluated. The therapeutic biomarkers of F-652 in this patient population are also investigated. F-652 is recombinant IL-22 dimer consisting of two monomeric subunits each comprising a sequence shown in SEQ ID NO: 24.

Study design: multicenter, controlled, single-blind, between investigational drug in combination with conventional antiviral regimen, and placebo in combination with conventional antiviral regimen.

Arms: Patients with severe COVID-19 (e.g., severe pneumonia) due to SARS-CoV-2 infection is recruited, and randomly assigned to Experimental group (F-652+conventional antiviral regimen) and Control group (placebo+conventional antiviral regimen) in a ratio of 1:1. Patients are administered with either 30 μg/kg F-652 (Experimental group) or placebo (Control group) by intravenous infusion on Day 1 after randomization, and either 30 μg/kg F-652 (Experimental group) or placebo (Control group) by intravenous infusion on Day 8 and Day 15 after randomization, in addition to conventional antiviral regimen (lopinavir/ritonavir (Kaletra®) tablet+IFN-α inhalation).

Study process: pulmonary function improvement assessment (clinical symptoms and CIPS score), liver function assessment (MELD, LILLE score), acute physiology and chronic health assessment (APACHE II score) and acute kidney injury assessment (RIFLE classification of AKI) are measured at the time of patient screening, Day 7, Day 14 and Day 21. The investigator determines whether the patient can be discharged from the hospital based on laboratory test indicators on Day 14 or Day 21 indexes (e.g., whether SARS-CoV-2 nucleic acid is tested negative), improvement of lung functions, and various clinical indicators. If hospitalization is still required, the extended period will be recorded. The last visit is completed on Day 30 after randomization. Clinical prognosis and outcomes are evaluated by telephonic interviews on Day 90 after randomization.

Additional clinical indicators can include: change from baseline in respiratory rate; change from baseline in pulse rate; change from baseline in systolic blood pressure; change from baseline in diastolic blood pressure; change from baseline in body temperature; change from baseline in oxygen saturation; change from baseline in RR, QRS, PR, QT, and QTcF intervals, as measured by electrocardiogram (ECG); change from baseline in heart rate, as measured by electrocardiogram (ECG); and number of participants with clinical laboratory test abnormalities in hematology parameters.

Changes in the serum levels of C-reactive protein (CRP), serum amyloid A (SAA), TNF, IL-2, IL-6, IL-10, regenerating islet-derived protein 3 alpha (Reg3A), FIB, and EGFR are also measured.

Efficacy Objective

Primary efficacy endpoints: clinical recovery time (from the beginning of treatment to fever, respiratory rate, finger oxygen saturation recovering to normal level and cough relief for at least 72 hours); improvement of lung function (CPIS score) on Day 7, Day 14 and Day 21.

Secondary efficacy endpoints: improvement of liver function (MELD, Lille score) on Day 7, Day 14 and Day 21; 30-day survival rate; 30-day patient improvement rate; the number of patients transferred to ICU for treatment and observation; hospitalization period for ICU stay; patients' total hospital stay; evaluation of acute kidney injury on Day 7, Day 14 and Day 21; acute physiological and chronic health assessment on Day 7, Day 14 and Day 21; number and proportion of cases of organ failure; number and proportion of co-infection cases; improvement of coagulation function, total bilirubin, serum creatinine, creatinine clearance, etc.; decrease in gastrointestinal adverse events above Grade II according to CTCAE 5.0. Additional secondary outcome measures can include: time to clinical improvement, defined as a National Early Warning Score 2 (NEWS2) of <2 Maintained for 24 hours; time to improvement of at least 2 categories relative to baseline on a 7-Category Ordinal Scale of Clinical Status (Time Frame: From Baseline up to 60 days).

Safety Objective

Primary safety endpoints: adverse events, including incidence, type, relevance to the investigational drug, and severity.

Secondary safety endpoints: changes in physical examination and vital signs; changes in laboratory examination and 12-lead electrocardiogram (ECG), e.g. change from baseline in RR, QRS, PR, QT, and QTcF intervals, as measured by ECG.

Exploratory biomarker measures: changes in the serum levels of CRP, serum amyloid A (SAA), TNF, IL-2, IL-6, IL-10, Reg3A, FIB and EGFR. Additional biomarker measures can include the prevalence of Anti-Drug Antibodies (ADAs) at Baseline and incidence of ADAs during the study.

### Example 3. Study of Therapeutic Effects of Recombinant IL-22 Dimer (F-652) on Endothelial Dysfunction

Provided in this Example are results demonstrating that F-652 reduces endothelial dysfunction and protects the endothelial glycocalyx (“EGX”; a network of membrane-bound proteoglycans and glycoproteins covering the endothelium luminally, regulating endothelial permeability) in the context of lipopolysaccharide (LPS) injury. Also provided are results suggesting that the protective effect of F-652 is mediated by downregulation of the TLR4 pathway in endothelial cells. The TLR4 pathway is activated in the context of viral infection as well as LPS injury. (Olejnik, J., Hume, A. J., & Mühlberger, E. (2018). “Toll-like receptor 4 in acute viral infection: Too much of a good thing.” PLoS pathogens, 14(12), e1007390). Thus, the results provided herein support a role of IL-22 treatment in preventing or treating a virus-induced organ injury or failure in an individual (Minako Yamaoka-Tojo. “Endothelial glycocalyx damage as a systemic inflammatory microvascular endotheliopathy in COVID-19,” Biomed J. 2020; 43(5): 399-413).

### Methods

HUVEC Culture

Human umbilical vein endothelial cells (HUVEC) were purchased from the American Type Culture Collection. Cells were initially grown in 2% gelatin-coated 10-cm plastic dishes using M200 medium supplemented with low serum growth supplement (LSGS) and penicillin/streptomycin in a cell culture incubator at 37° C. with 5% CO₂ atmosphere. Cells were passaged by digestion in 0.25% trypsin in Hanks' Balanced Salt Solution (HBSS) after reaching 80% confluence. Cells were used for experiments between passages 1-3. For glycocalyx quantification, HUVECs were plated in 48-well plastic cell culture plates coated with 2% gelatin, at a confluence of approximately 80%. M200+LSGS+penicillin/streptomycin was supplemented with 1% bovine serum albumin (BSA) to support glycocalyx growth. Cells were cultured for 24 hours to allow glycocalyx development before LPS exposure.

Experimental Design

To investigate the effects of F-652 on EGX, cultured HUVECs were exposed to either untreated media, 1 μg/mL of LPS, 1 μg/mL of LPS and 0.375 μg/mL of F-652, or 0.375 μg/mL of F-652 alone, for a total of 24 hours.

Glycocalyx Quantification

After completion of the LPS exposure with or without F-652, HUVECs were fixed by addition of concentrated formaldehyde solution directly to the culture medium to yield a final formaldehyde concentration of 3.5%. After 10 minutes of fixation, cells were washed with phosphate-buffered saline (PBS) supplemented with 1% BSA. Cells were then stained with 23 μg/mL WGA and 23 μg/mL 4′,6-diamidino-2-phenylindole in PBS with 1% BSA for 20 minutes at room temperature in the dark. Staining was performed for this short period to ensure no penetration of the WGA into the cytoplasm, confounding results with non-surface layer staining. Cells were then washed twice with 1% BSA in PBS and covered with Fluoro-Gel mounting medium (Electron Microscopy Sciences). Glycocalyx and nuclei (4′,6-diamidino-2-phenylindole) were imaged on an EVOS fluorescence microscope under identical conditions. Three images were taken of each condition, with approximately 100 cells per image. ImageJ software was used to quantify glycocalyx fluorescence intensity overlaying the nuclei of each visible cell.

Measuring the IL-22Ra1 Receptor with Immunofluorescence

HUVECs were fixed in 3.5% formaldehyde in PBS for 10 minutes. Cells were then blocked in 1% BSA in PBS for one hour. Cells were then incubated overnight in primary antibody for IL-22Ra1 (Invitrogen, Carlsbad, Calif.) diluted 1:100 in 1% BSA in PBS. Cells were then washed with PBS 3 times. Cells were incubated with secondary antibody, goat anti-mouse Alexa Fluor 488 (1:500; Invitrogen, A28175) diluted 1:500 in 1% BSA in PBS along with 0.1 μg/ml of 4,6 diamidino-2phylindole (DAPI) (Sigma) for one hour, followed by three washes in PBS. Cells were then cover slipped with Fluoro Gel mounting medium and imaged on an EVOS fluorescence microscope. Fluorescence intensity was quantified using ImageJ.

SDS-Polyacrylamide Gel Electrophoresis Western Blots for Total STAT3 and Phosphorylated STAT3

HUVECs were lysed in lysis buffer (50 mM Tris-HCl pH 7.5, 150 mM NaCl, 0.5 M EDTA, 1% Triton X-100, and Halt™ protease inhibitor cocktail). Proteins were quantified using Bio-Rad protein quantification assay (Bio-Rad Laboratories), and 20-50 μg of protein was separated by SDS-polyacrylamide gel electrophoresis (SDS-PAGE) on a 4-12% gradient acrylamide gel run at 100 V. Proteins were then transferred to 0.45 m PVDF membrane at 30 V for 2 hours. Membranes were blocked in Tris Buffered Saline (TBS: 137 mM NaCl, 20 mM Tris Base), 0.1% Tween 20, and 5% bovine serum albumin (blocking solution) for 1 hour, followed by overnight incubation with primary antibody diluted in TBS, 0.1% Tween 20, and 3% BSA, and 1 hour incubation with horseradish peroxidase-conjugated secondary antibody diluted at 1:5,000. The primary antibody used for signal transducer and activator of transcription 3 (STAT3) was rabbit monoclonal antibody #30835S (Cell Signaling Technology) and the primary antibody for phosphorylated STAT3 (p-STAT3) was rabbit monoclonal antibody #9145 (Cell Signaling Technology). Immunoreactive protein was detected using ECL (GE Healthcare) imaged on a Bio-Rad ChemiDoc™ MP Imaging System.

Real-Time Quantitative Reverse Transcription PCR

RNA was isolated with Trizol (Invitrogen) and used as a template for reverse transcriptase (reverse transcriptase mix sold under the trademark ISCRIPT® RT supermix, Bio-Rad). mRNAs were quantified by real-time PCR with the cyanine nucleic acid dye IQ SYBR® Green Supermix (Bio-Rad), and normalized against PPIA mRNA as the internal control gene. Relative changes in expression were calculated using the AACt method as established in prior studies. (Livak K J, Schmittgen T D. “Analysis of relative gene expression data using real-time quantitative PCR and the 2^{-ΔΔCT} method.” Methods. 2001; 25(4):402-8).

Statistical Analysis

Glycocalyx staining intensity and RNA levels were presented as means±standard error and difference between groups was analyzed by Student's t test. A p-value of less than 0.05 was considered significant for all tests.

### Results

Glycocalyx Shedding

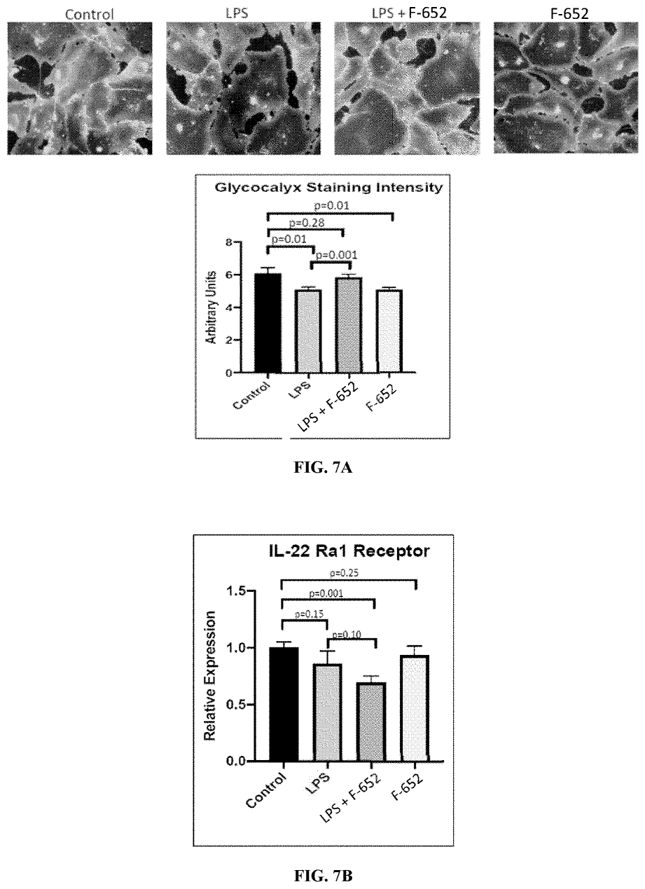

A comparison of glycocalyx intensity is shown in FIG. 7A. When compared to control, LPS exposure led to glycocalyx degradation (6.09 [control] vs. 5.10 [LPS] Arbitrary Unit [AU], p=0.01). However, exposure to LPS and F-652 did not result in glycocalyx degradation as compared to control (6.09 [control] vs. 5.86 [LPS+F-652] AU, p=0.28). HUVECs exposure to F-652 alone resulted in glycocalyx shedding compared to control (6.09 [control] vs. 5.08 [F-652] AU, p=0.01). Glycocalyx shedding was worse in HUVECs exposed to LPS alone as compared to LPS with F-652 (5.10 [LPS] vs. 5.86[LPS+F-652] AU, p=0.001). Representative images of fluorescent microscopy are shown for all 4 groups in FIG. 7A.

IL-22Ra1 Receptor and STAT3 Signaling

Interleukin 22 receptor, alpha 1 (IL-22Ra1) is one of the two subunits of IL-22 receptor. As shown in FIG. 7B, exposure to LPS (p=0.15) or F-652 (p=0.25) alone did not result in a difference in the IL-22Ra1 receptor expression compared to control. Exposure to LPS and F-652 did result in a decrease in IL-22Ra1 receptors (1.00 [control] vs. 0.69 Relative Expression [RE], p=0.001). IL-22Ra1 receptor relative expression was not significantly different in HUVECS with LPS only exposure compared to HUVECS with LPS and F-652 exposure (p=0.10).

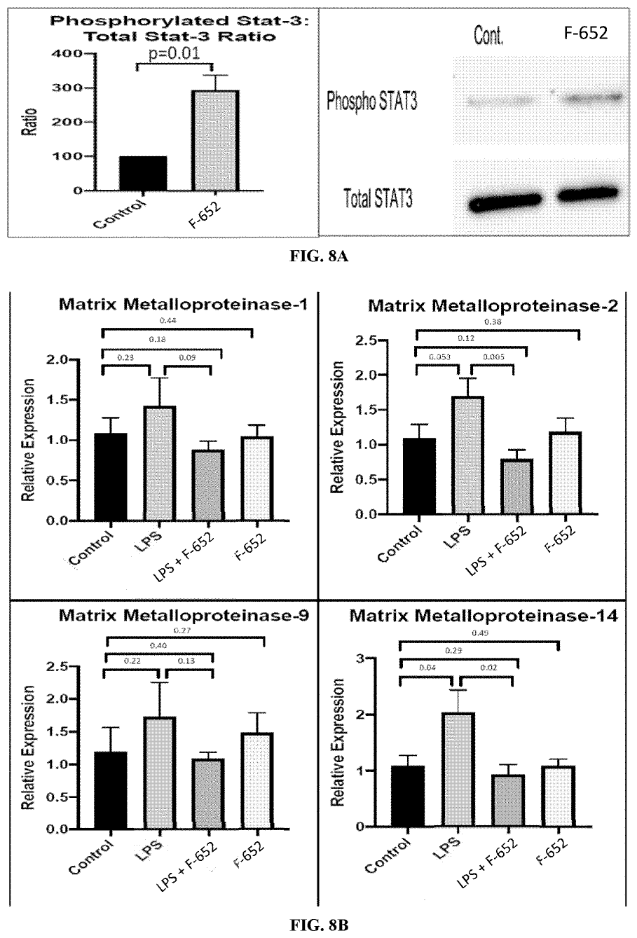

FIG. 8A shows that the ratio of phosphorylated STAT3 to total STAT3 in control HUVECs compared to HUVECs exposed to F-652 alone. The ratio of phosphorylated STAT3 to total STAT3 in the F-652 treated is significantly higher in the F-652 treated HUVECs as compared to control (p=0.01). A representative image of an SDS-Polyacrylamide gel electrophoresis western blot quantifying phosphorylated STAT3 and total STAT3 is shown in the right panel of FIG. 8A.

Metalloproteinases

Matrix metalloproteinase (MMP) has crucial roles in immune responses. Active MMPs modify immune substrates or cleave transmembrane receptors, thereby affecting cell-cell communication and intracellular signaling. MMPs are capable of disrupting endothelial cell surface proteins, such as syndecans, resulting in derangements of the EGX.

Treatment of HUVECs with LPS (p=0.23) or LPS and F-652 (p=0.18) did not significantly change the expression of Matrix Metalloproteinase-1 (MMP-1) compared to control. HUVECs exposed to LPS had higher levels of MMP-2 (p=0.053) and MMP-14 (p=0.04) as compared to controls; while exposure of HUVECs with to LPS and F-652 resulted in lower relative expression of MMP-2 (p=0.12) and MVP-14 (p=0.29) as compared to control. Treatment of HUVECs with LPS (p=0.22) or LPS and F-652 (p=0.40) did not change the expression of MMP-9 as compared to control. Treatment with F-652 only did not change levels of any matrix metalloproteinase. See, FIG. 8B.

MMP-7 levels did not change compared to control when treated with LPS (1.11 [control] vs. 2.99 RE, p=0.06), LPS and F-652 (1.11 [control] vs. 1.53 RE, p=0.15), or F-652 alone (1.11 [control] vs. 1.23 RE, p=0.38). MMP-9 relative expression was not different when LPS exposed HUVECs were compared to LPS and F-652 exposed HUVECs (2.99 vs. 1.53 RE, p=0.09). In addition, A Disintegrin And Metalloproteinase (ADAM) domain 17 (ADAM17) levels did not change compared to control when treated with LPS (1.09 [control] vs. 2.42 RE, p=0.06), LPS and F-652 (1.09 [control] vs. 1.22 RE, p=0.31), or F-652 alone (1.09 [control] vs. 1.14 RE, p=0.42). ADAM17 relative expression was not significantly different when LPS exposed HUVECs were compared to LPS and F-652 exposed HUVECs (2.42 vs. 1.22 RE, p=0.054).

Pro-Glycocalyx Agents

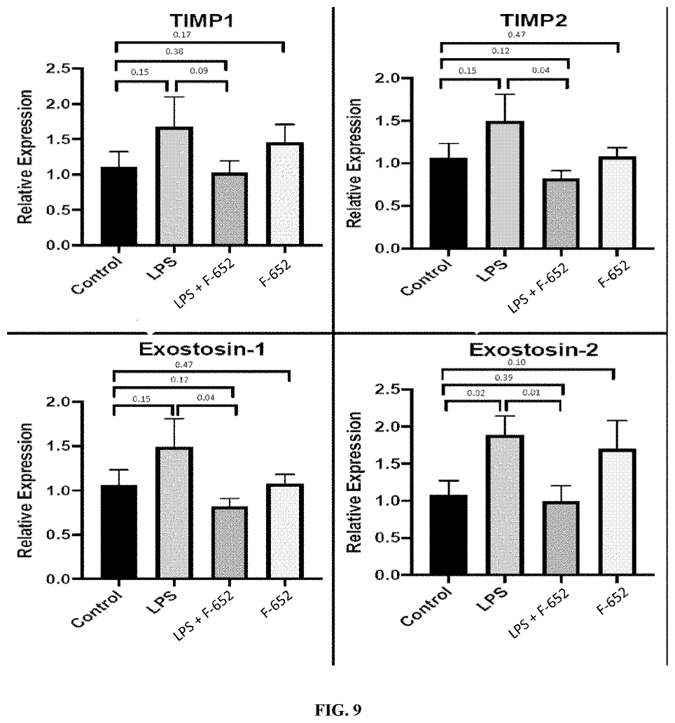

Inhibition of MVPs occurs naturally by a class of tissue inhibitors of metalloproteinases (TIMPs). Tissue inhibitor of metalloproteinase-1 (TIMP1) was not different among various HUVEC exposure groups. When compared to LPS exposure only, TIMP2 level was lower in LPS and F-652 co-exposed HUVECs (1.49 vs. 0.82 RE, p=0.04). All other comparisons for TIMP2 were not significantly different (LPS vs. control; LPS+F-652 vs. control; or F-652 vs. control). Exostosin-1 is involved in EGX reconstitution. Exostosin-1 (1.49 vs. 0.82 RE, p=0.04) and Exostosin-2 (1.88 vs. 0.99 RE, p=0.01) levels were significantly higher in LPS only exposed HUVECs as compared to LPS and F-652 co-exposed HUVECs. Exostosin-2 level was significantly higher in LPS only exposed HUVECs as compared to control (1.88 vs. 1.08 RE, p=0.02). See, FIG. 9.

Vascular Endothelial Cadherin Levels

Vascular endothelial cadherin (VE-CAD) is a membrane protein that is the major component of adherens junctions between endothelial cells. It is crucial for regulating vascular integrity, endothelial permeability, and angiogenesis. During inflammatory processes, VE-CAD is shed into circulation (sVE-CAD). VE-CAD RNA levels were higher in LPS only exposed HUVECs as compared to control (1.96 vs. 1.06 RE, p=0.048). LPS only treated HUVECs had significantly higher VE-CAD RNA levels than LPS and F-652 co-exposed HUVECs (1.96 vs. 0.81 RE, p=0.02). VE-CAD in LPS and F-652 co-exposed (1.06 [control] vs. 0.81 RE, p=0.18) and F-652 only exposed (1.06 [control] vs. 1.01 RE, p=0.41) HUVECs were not significantly different than control.

Toll-Like Receptor 4 Signaling Pathway

Toll-like Receptor 4 (TLR4) recognizes bacterial LPS. Myeloid differentiated primary response 88 (MyD88) is utilized by TLR4 and activates NF-xB and MAPKs for the induction of inflammatory cytokine genes. Toll-interleukin-1 receptor domain containing adapter protein (TIRAP) is a sorting adaptor that recruits MyD88 to TLR4. MyD88 recruits interleukin-1 receptor associated kinase 1 (IRAK-1), IRAK-4, and then TNF receptor-associated factor 6 (TRAF6), resulting in the nuclear translocation of the prototypic inflammatory transcription factor NF-κB. TIR domain-containing adapter protein inducing IFNβ (TRIF) mediates the MyD88-independent pathway leading to TLR4-mediated activation of the transcription factor interferon regulatory factor 3, which regulates Type I IFN production. The TRIF-related adapter molecule (TRAM) specifically acts to bridge TLR4 with TRIF. See B. Verstak et al. (J Biol Chem. 2009; 284(36): 24192-24203).

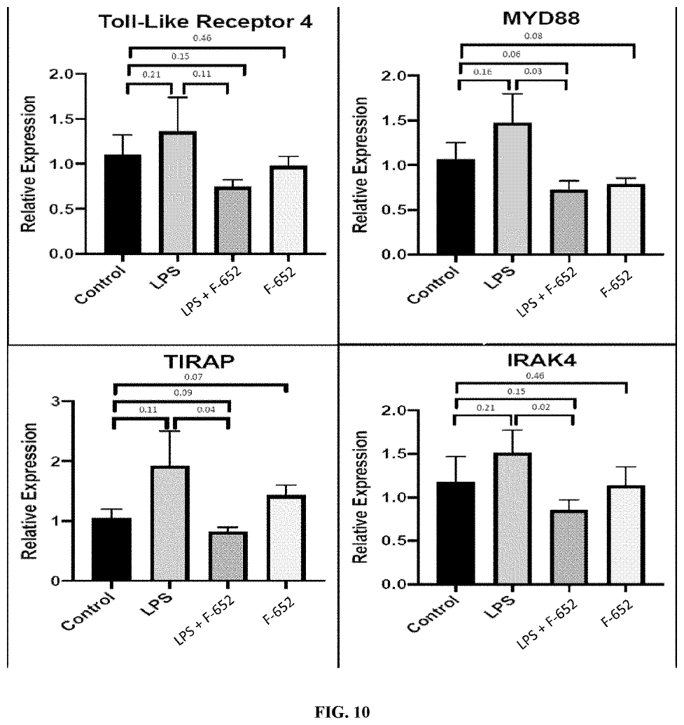

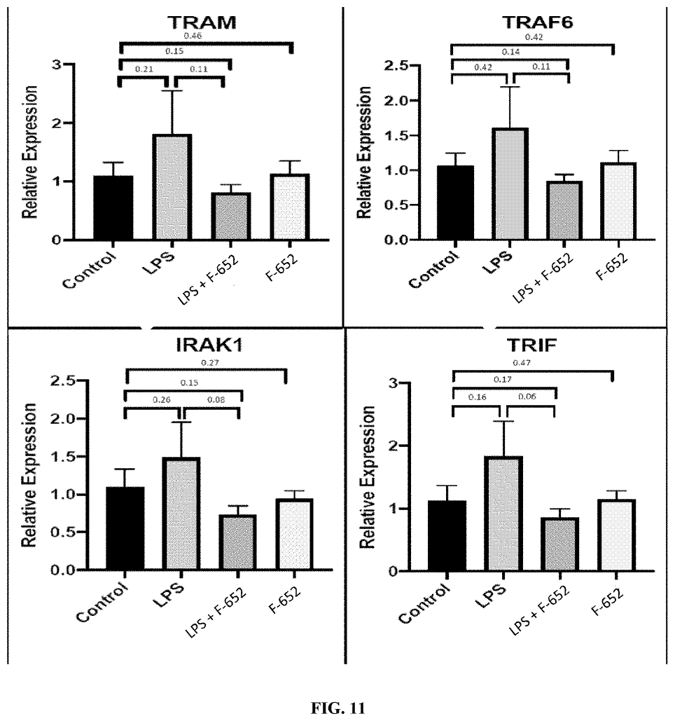

TLR4 mRNA was not significantly different in all comparisons (FIG. 10). IYD88 RNA expression was lower in LPS and F-652 co-exposed HUVECs as compared to LPS only exposed HUVECs (0.72 vs. 1.48 RE, p=0.03). All other comparisons (LPS vs. control; LPS+F-652 vs. control; or F-652 vs. control) of MYD88 were not significantly different. Similarly, TIRAP mRNA expression was lower in LPS and F-652 co-exposed HUVECs as compared to LPS only exposed HUVECs (0.82 vs. 1.92 RE, p=0.04), but not significantly different in all other comparisons. In addition, IRAK4 mRNA expression was lower in LPS and F-652 co-exposed HUVECs as compared to LPS only exposed HUVECs (0.86 vs. 1.51 RE, p=0.02), but not significantly different in all other comparisons. See, FIG. 10. Levels of TRAM, TRAF6, IRAK1, and TRIF were not significantly different in all group comparisons as shown in FIG. 11.

### DISCUSSION

Endothelial dysfunction and glycocalyx shedding are notable sequelae of virus-induced injury caused by viruses such as coronaviruses (Okada, H, Yoshida, S, Hara, A, Ogura, S, Tomita, H. “Vascular endothelial injury exacerbates coronavirus disease 2019: The role of endothelial glycocalyx protection.” Microcirculation. 2020; 00:e12654). The endothelial glycocalyx (EGX) can be degraded via several inflammatory mechanisms, including sheddases such as metalloproteinases, heparanases, and hyaluronidases. This contributes to vascular hyper-permeability, microvascular thrombosis, and enhanced leukocyte adhesion. In this Example, we provide results demonstrating that F-652 protects against shedding of the EGX after LPS injury. Additionally, we provide results demonstrating that F-652 may reduce EGX shedding via downregulation of the TLR4 signaling pathway. These results support a therapeutic role for F-652 in treating virus-induced organ injury or failure in an individual.

Provided herein are results showing that F-652 has a protective effect on the EGX. Interestingly, treating the EGX with F-652 alone led to EGX shedding, however, in the context of endothelial injury (LPS treatment), F-652 preserved the EGX layer with respect to control (FIG. 7A).

MMPs are upregulated in various models acute lung injury and acute respiratory distress syndrome (ALI/ARDS). In addition, MMPs play a key role in degradation of the EGX. We found that F-652 resulted in a statistically significant decrease in expression of MMP-2 and MMP-14 in cells treated with LPS, which would otherwise induce endothelial dysfunction. The results also suggest that F-652 may decrease expression of MMP-1 and MMP-9 in cells treated with LPS, which would otherwise induce endothelial dysfunction, although further experiments are required to confirm the significance of this decrease (FIG. 8B).

While F-652 co-exposure with LPS did not decrease TLR4 expression, it did down-regulate multiple mediators of this pro-inflammatory pathway. MYD88, TIRAP, and IRAK4 are all key mediators in the TLR4 pathway that were decreased in the presence of LPS and F-652. These results provide evidence that IL-22 can decrease the expression of TLR4 mediators. Down-regulation of this pathway may explain the decrease in MMP-2 and MMP-9 that was observed in the present study. Furthermore, this finding highlights the potential for F-652 to be a novel therapeutic in severe infection.

In conclusion, this study demonstrates that F-652 alone induces EGX degradation, however, in the presence of injury (e.g. LPS injury), F-652 mitigates EGX degradation. IL-22Ra1 receptors are present on endothelial cells and signal through the phosphorylated-STAT3 pathway. The protective effect of F-652 to the EGX appears to be mediated via reducing metalloproteinases and down-regulation of the TLR4 pathway. These findings suggest a potential therapeutic effect of F-652 in the endotheliopathy that occurs in severe viral infection (e.g., coronavirus infection) or sepsis.

### Example 4. Study of Therapeutic Effects of Recombinant IL-22 Dimer (F-652) on Endothelial Dysfunction in a Mouse Model of Acute Lung Injury

Provided in this example are results establishing proof of concept that F-652 may have a therapeutic benefit in a pre-clinical model of ARDS, such as in viral infection.

### Methods

Acute Lung Injury and F-652 Treatment

After approval from the Tulane University, Institutional Animal Care and Use Committee (protocol ID 607), equal numbers of male and female, 6-8 week old C57BL/6 mice (Charles River Laboratories, Cambridge, Mass.) were given acute lung injury (ALI) via intra-tracheally administered LPS. After obtaining appropriate depth of anesthesia using isoflurane, the high-dose LPS group (HDG) received 100 μg of LPS administered intra-tracheally. Approximately 30 minutes after LPS administration, 4 μg of F-652 was administered via tail vein injection (n=11), then compared to animals receiving sham injection (n=8) with phosphate-buffered saline (PBS). In the low-dose LPS group (LDG), 33.3 μg of LPS was administered intra-tracheally. F-652 was again administered at 30 minutes (n=9) and compared to sham injected animals (n=9). The Interleukin-22:Fc (F-652) protein is a recombinant fusion protein (F-652) (Evive Biotech, Shanghai, China) with two human IL-22 molecules linked to the Fc portion of human immunoglobulin G2, which extends the half-life of the molecule.

Evaluation of Lung Injury

Euthanasia and bronchoalveolar lavage (BAL) was carried out on post-injury day 4. After obtaining appropriate levels of anesthesia with inhaled isoflurane, the trachea was cannulated using a 26 gauge needle and BAL was performed with three successive washes using 1 mL of PBS. Next, a small segment of the left lower lobe was removed and saved for RNA isolation. Finally, 1 cc of 4% paraformaldehyde was injected to the lung for fixation.

The BAL fluid was then centrifuged at 500×gravity for 5 minutes. Cells were obtained from the BAL after centrifuge and cell counts performed. Cells were then affixed to glass slides and stained with Wright's stain. To quantify protein in the BAL supernatant, a Bradford protein assay (Bio-Rad Laboratories) was performed. Protein was quantified by measuring absorbance at 595 nm on a BMG Labtech FLUOstar Optima plate reader. In addition, the BAL supernatant was used to measure pro-inflammatory cytokines using a Milliplex Mouse Cytokine/Chemokine Magnetic Bead Panel (Millipore Sigma). The 32 cytokines measured included Eotaxin, Granulocyte Colony-Stimulating Factor (G-CSF), Granulocyte-Monocyte Colony-Stimulating Factor (GM-CSF), Interferon-γ (IFN-γ), Interleukin-1α (IL-1α), IL-1, IL-2, IL-3, IL-4, IL-5, IL-6, IL-7, IL-9, IL-10, IL-12 (p40 segment), IL-12 (p70 segment), IL-13, IL-15, IL-17, Interferon-γ induced protein 10 (IP-10), keratinocyte chemoattractant (KC), leukemia inhibitory factor (LIF), lipopolysaccharide-induced CXC chemokine (LIX), monocyte chemoattractant protein-1 (MCP-1), macrophage colony-stimulating factor (M-CSF), monokine induced by gamma-interferon (MIG), MIP-1α, macrophage inflammatory protein-1β/CCL4 (MIP-1β), MIP-2, regulated upon activation, normal T-cell expressed and presumably secreted (RANTES), tumor necrosis factor-α (TNF-α), vascular endothelial growth factor (VEGF). Human IL-22 was measured using an IL-22 Human ELISA kit (ThermoFisher Scientific). Mouse IL-22 was measured using an IL-22 Mouse/Rat Quantikine ELISA kit (R&D Systems).

Histopathological Evaluation

Immediately after sacrifice, lung tissue from the right lower lobe was fixed in 4% paraformaldehyde and cut into sections. The sections were stained with hematoxylin and eosin (H&E). Lung injury induced by LPS was assessed by a blinded reviewer with a numerical scoring scale ranging from 0-4. Regions of lung injury in sections were scored for the extent of intimal thickening, alveolitis, and the presence of proteinaceous material in the alveolar space. Representative images were taken.

Endothelial Glycocalyx Measurements

Paraformaldehyde-fixed lung segments were flash-frozen in Optimal Cutting Temperature (O.C.T) compound (Sakura) and sectioned on a cryostat. Sections were then blocked with PBS supplemented with 1% BSA. Tissue was then stained with 23 μg/mL WGA and 23 μg/mL 4′,6-diamidino-2-phenylindole in PBS with 1% BSA for one hour at room temperature in the dark. Sections were then washed three times with PBS and covered with Fluoro-Gel mounting medium (Electron Microscopy Sciences). Glycocalyx and nuclei (4′,6-diamidino-2-phenylindole) were imaged on an Olympus BX51 fluorescence microscope under identical conditions. ImageJ software was used to quantify glycocalyx fluorescence intensity in the alveolar capillaries from a minimum of 20 regions of interest from 3 mice per condition.

Immunofluorescence Stains

Lung tissue was fixed in 4% paraformaldehyde in PBS overnight. Paraformaldehyde-fixed lung segments were flash-frozen in Optimal Cutting Temperature (O.C.T.) compound (Sakura) and sectioned on a cryostat. Tissue was then blocked in 1% BSA in PBS for one hour. Tissue was then incubated overnight in primary antibody for IL-22Ra1 (Invitrogen, Carlsbad, Calif.) and E-cadherin (Sigma) diluted 1:100 in 1% BSA in PBS. Cells were then washed with PBS 3 times. Cells were incubated with secondary antibody, goat anti-mouse Alexa Fluor 488 (1:500; Invitrogen, A28175) and goat anti-rabbit Alexa Fluor 555 (1:500, Invitrogen, A27039) diluted in 1% BSA in PBS along with 0.1 μg/ml of 4,6 diamidino-2phylindole (DAPI) (Sigma) for one hour, followed by three washes in PBS. Cells were then cover slipped with Fluoro Gel mounting medium and imaged on an Olympus BX51 fluorescence microscope. Fluorescence intensity was quantified using ImageJ.

RNA-seq

Lung tissue was homogenized in Trizol buffer (Life Technologies) and total RNA extraction was performed according to Trizol manufacturer's instructions. Total RNA was used to perform RNA sequencing (RNA-seq). RNA quantity and quality were assessed using NanoDrop and Agilent RNA ScreenTape with Agilent 4150 TapeStation system. SMART-Seq Stranded total RNA sample prep kit (Takara Bio USA, Inc.) was used for library preparation as specified in the user manual, followed by Agilent DNA 1000 kit validation with Agilent 4150 TapeStation system and quantification by Qubit 2.0 fluorometer. The cDNA libraries were pooled at a final concentration 1.2 μM. Cluster generation and 1×75 bp single read single-indexed sequencing was performed by High-Output kit v2.5 (75 cycles) on Illumina NextSeq 550. Raw reads were processed and mapped. Pathway analysis was performed using Advaita Bioinformatics Genomics Workbench.

Statistical Analysis

Values were presented as means±standard error and difference between groups was analyzed by Student's t test. A p-value of less than 0.05 was considered significant for all tests.

### Results

Cell Counts Measured in BAL

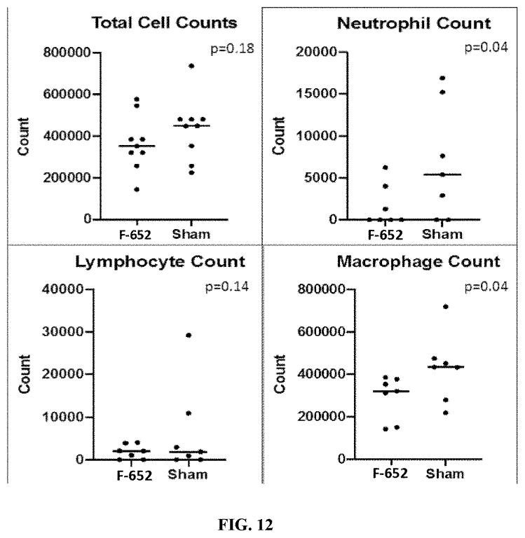

To examine the degree of inflammatory cell influx in high dose injury animals, we compared cell counts between F-652 treated animals and sham animals. Cell counts for low-dose LPS injured animals are shown in FIG. 12. Total cell counts were not significantly different in F-652 treated animals when compared to sham animals (364,444 vs. 433,889 cells, p=0.18). Neutrophil count was significantly lower in the F-652 treated animals compared to sham animals (1,653 vs. 6,869 cells, p=0.04). Lymphocyte count was not significantly different in F-652 treated animals and sham animals (1,864 vs. 6,556 cells, p=0.14), however, macrophage count was significantly lower (290,611 vs. 429,262 cells, p=0.04) in F-652 treated animals. See, FIG. 12.

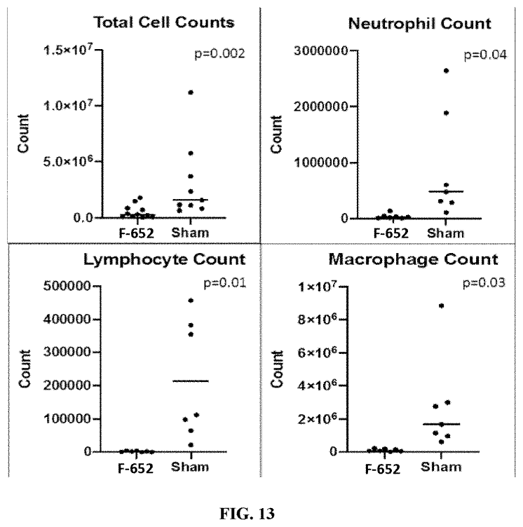

A comparison of cell counts for high-dose LPS injury is shown in FIG. 13. Mice treated with F-652 had significantly lower total cell counts (5.40×10⁵ vs. 3.15×10⁶ cells, p=0.002), significantly lower neutrophil counts (3.69×10⁴ vs. 8.99×10⁵ cells, p=0.04), significantly lower lymphocyte counts (2,163 vs. 213,225 cells, p=0.01), and significantly lower macrophage counts (1.21×10⁵ vs. 2.72×10⁶ cells, p=0.03) compared to sham animals.

BAL Inflammatory Mediators

To examine the degree of inflammation in lungs after F-652 treatment, we compared inflammatory mediators in BAL fluid of treated and untreated sham animals. A comparison of all inflammatory mediators measured in the BAL of mice with low-dose LPS injury is shown in Table 3. There was no significant difference in the amount of any measured inflammatory mediators when comparing the F-652 treated to sham animals.

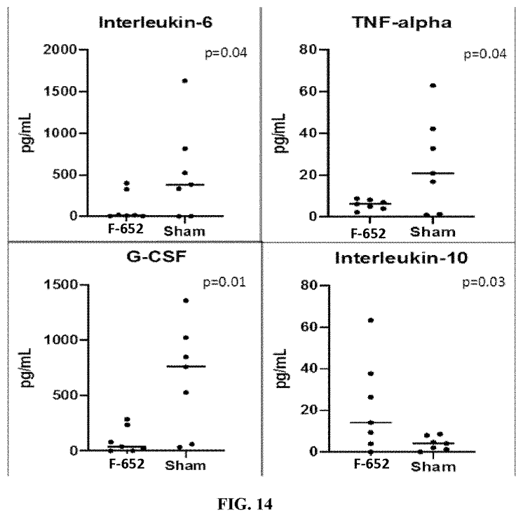

Inflammatory mediators in high-dose LPS injured animals are shown in FIG. 14. IL-6 (110.6 vs. 527.1 pg/mL, p=0.04), TNF-α (5.87 vs. 25.41 pg/mL, p=0.04), and G-CSF (95.14 vs. 659.6, p=0.01) levels were all significantly lower in the BAL fluid of F-652 treated animals compared to sham controls. Interleukin-10 levels in BAL fluid were significantly higher in F-652 treated animals compared to sham animals (22.10 vs. 4.05 pg/mL, p=0.03). A summary of all other cytokines measured in the multiplex assay is shown in Table 4. IL-1α, IL-2, IL-5, IL-9, IL-12, IL-15, and M-CSF were found to have significantly lower levels in the F-652 treated animals compared to sham animals.

Protein Leak and Histopathology Scores

To examine the degree of lung leak and lung damage, we measured BAL protein levels and compared histopathology scores. After low-dose LPS injury, BAL protein in animals receiving F-652 was significantly lower than sham animals (0.15 vs. 0.25 μg/μL, p=0.03). A comparison of histopathology scores among animals with low dose LPS injury did not show any difference between F-652 treated and sham animals.

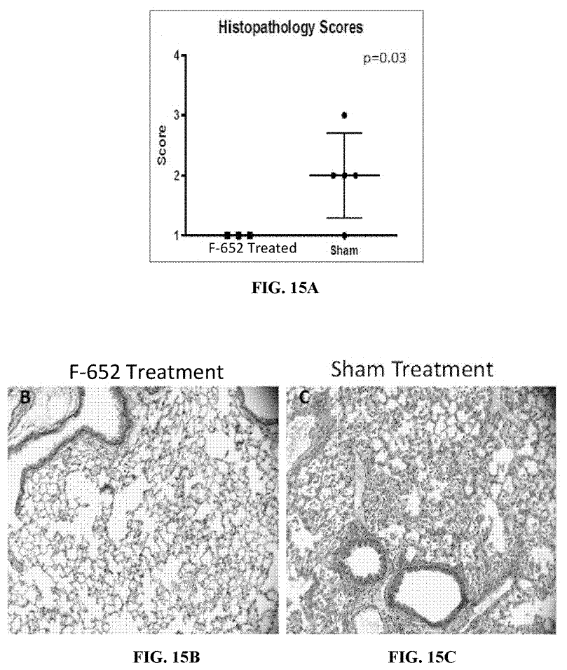

After high-dose LPS injury, BAL protein in animals receiving F-652 was not different compared to sham animals (0.55 vs. 0.38 μg/μL, p=0.18). A comparison of histopathology scores of high-dose LPS inured animals (FIG. 15A) showed that F-652 treated animals had significantly less severe injury scores (1.0 vs. 2.0, p=0.03). Representative histopathological images of F-652 treated and sham animals are shown in FIG. 15B and FIG. 15C, respectively.

Glycocalyx Degradation

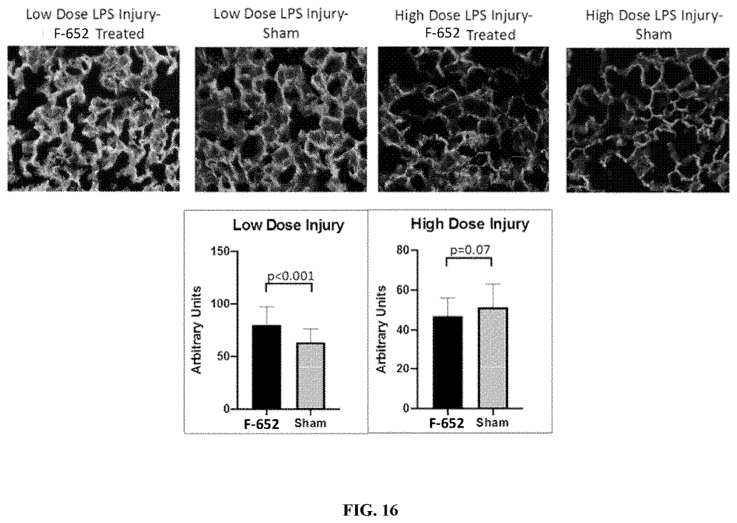

To determine if F-652 helps maintain the glycocalyx layer in the endothelium of alveolar capillaries, endothelial glycocalyx intensity was measured as seen in FIG. 16. In the low dose LPS injury group, F-652 resulted in significantly greater intensity of the glycocalyx (80.0 vs. 63.7 Arbitrary Units, p<0.001) after LPS injury. Images of glycocalyx staining are shown in FIG. 16. In the high dose LPS injury group, there was no significant difference in glycocalyx intensity when comparing F-652 treated with sham animals (p=0.07).

Exogenous vs. Endogenous IL-22

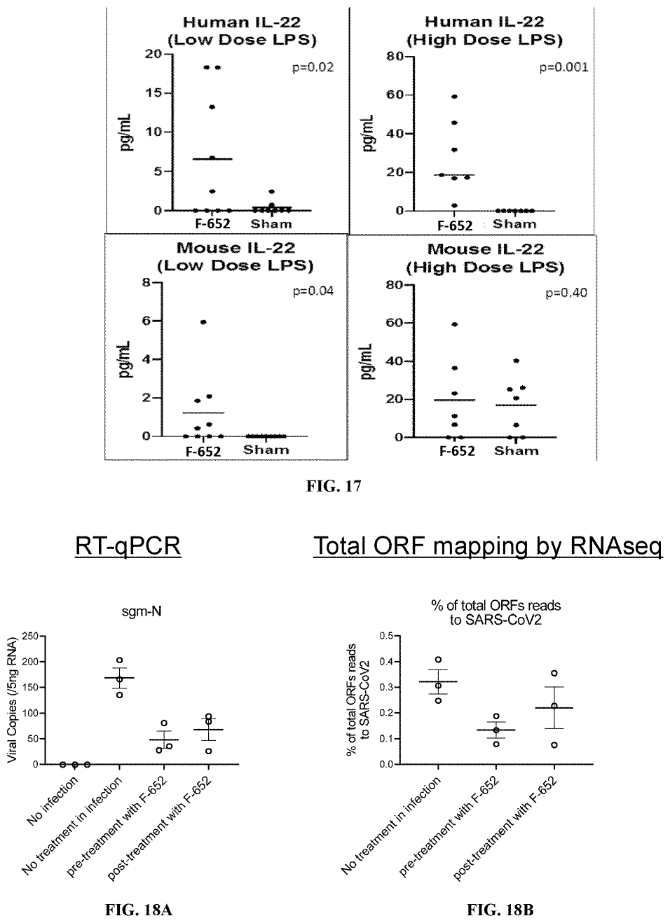

To determine if the effect on the lungs was due to exogenous F-652 or endogenous IL-22, human and mouse IL-22 was measured in the BAL of both high and low dose LPS injured animals. As shown in FIG. 17, there were significantly higher levels of human IL-22 in the F-652 treated animals in both low-dose LPS (6.56 vs. 0.40 μg/mL, p=0.02) and high-dose LPS (27.41 vs. non-detectable pg/mL, p=0.001) injured animals. Endogenous mouse IL-22 levels in the low-dose LPS injury group was higher in the F-652 treated animals (1.22 vs. non-detectable pg/mL, p=0.04). However, endogenous IL-22 was not different in the high-dose LPS injury animals treated with F-652 (19.57 vs. 17.02 μg/mL, p=0.40) compared to sham. See, FIG. 17.

RNA-seq Analysis

Pathway analysis of gene expression showed that the cytokine-cytokine receptor pathway was significantly different in F-652 treated animals after high-dose LPS injury. F-652 treatment resulted in a decrease in Macrophage Inflammatory Protein-1β (CCL4) expression (p=0.01). Differentially expressed pathway genes for extracellular matrix-receptor interactions were also different between groups. Tenascin C (Tnc), collagen, type I, alpha 1 (COL1a1), collagen, type VI, alpha 3 (Col6a3), and collagen, type I, alpha 2 (Col1a2) expression was increased with F-652 treatment (p=0.003).

TABLE 3
A comparison of F-652 Treated and Sham Animals
After Acute Lung Injury with Low Dose LPS

| | F-652 Treated | Sham | |
|---|---|---|---|
| Cytokine | (μg/mL) | (μg/mL) | p-value |
| Interleukin-1α | 61.31 ± 12.36 | 47.00 ± 9.64 | 0.19 |
| Interleukin-1β | 0.01 ± 0.01 | 0.01 ± 0.01 | 0.50 |
| Interleukin-2 | 6.77 ± 1.75 | 3.37 ± 1.11 | 0.06 |
| Interleukin-3 | 0.00 ± 0.00 | 0.00 ± 0.00 | 0.99 |
| Interleukin-4 | 0.00 ± 0.00 | 0.00 ± 0.00 | 0.99 |
| Interleukin-5 | 0.00 ± 0.00 | 0.19 ± 0.19 | 0.17 |
| Interleukin-6 | 0.95 ± 0.62 | 2.39 ± 1.47 | 0.19 |
| Interleukin-7 | 0.00 ± 0.00 | 0.00 ± 0.00 | 0.99 |
| Interleukin-9 | 154.50 ± 37.46 | 120.40 ± 37.64 | 0.27 |
| Interleukin-10 | 19.91 ± 6.56 | 13.60 ± 6.53 | 0.25 |
| Interleukin-12 (p40) | 6.87 ± 1.88 | 5.83 ± 1.96 | 0.35 |
| Interleukin-12 (p70) | 0.00 ± 0.00 | 0.00 ± 0.00 | 0.99 |
| Interleukin-13 | 2.69 ± 0.93 | 1.35 ± 0.88 | 0.16 |
| Interleukin-15 | 0.00 ± 0.00 | 0.00 ± 0.00 | 0.99 |
| Interleukin-17 | 0.40 ± 0.06 | 0.45 ± 0.08 | 0.32 |
| Tumor Necrosis Factor-α | 3.01 ± 1.79 | 3.01 ± 1.76 | 0.50 |
| Eotaxin | 2.59 ± 1.93 | 7.96 ± 3.65 | 0.11 |
| Interferon-γ | 1.15 ± 0.42 | 2.28 ± 1.17 | 0.19 |
| Granulocyte Colony-Stimulating Factor | 23.49 ± 9.15 | 33.76 ± 9.96 | 0.23 |
| Granulocyte-Macrophage Colony- | 0.00 ± 0.00 | 0.00 ± 0.00 | 0.99 |
| Stimulating Factor | | | |
| Interferon-γ-Induced Protein-10 | 12.94 ± 3.54 | 25.88 ± 9.82 | 0.12 |
| Keratinocyte Chemoattractant/Growth | 9.11 ± 2.76 | 10.72 ± 2.71 | 0.34 |
| Regulated Oncogene | | | |
| Monocyte Chemoattractant Protein | 0.00 ± 0.00 | 0.00 ± 0.00 | 0.99 |
| Macrophage Inflammatory Protein-1α | 15.62 ± 2.03 | 19.89 ± 11.37 | 0.19 |
| Macrophage Inflammatory Protein-1β | 0.00 ± 0.00 | 2.15 ± 2.15 | 0.17 |
| Macrophage Inflammatory Protein-2 | 20.45 ± 10.88 | 11.81 ± 5.57 | 0.25 |
| Monocyte Induced by Interferon-γ | 10.52 ± 4.73 | 26.22 ± 14.25 | 0.16 |
| Macrophage Colony-Stimulating Factor | 0.00 ± 0.00 | 0.00 ± 0.00 | 0.99 |
| C-X-C Motif Chemokine 5 (LIX) | 9.27 ± 6.04 | 0.76 ± 0.76 | 0.09 |
| Vascular Endothelial Growth Factor | 3.01 ± 0.50 | 3.17 ± 1.45 | 0.42 |

TABLE 4
A comparison of F-652 Treated and Sham Animals
After Acute Lung Injury with High Dose LPS

| | F-652 Treated | Sham | |
|---|---|---|---|
| Cytokine | (μg/mL) | (μg/mL) | p-value |
| Interleukin-1α | 69.39 ± 5.91 | 28.14 ± 5.40 | <0.001 |
| Interleukin-1β | 3.48 ± 1.25 | 10.05 ± 3.26 | 0.04 |
| Interleukin-2 | 7.33 ± 0.83 | 0.26 ± 0.45 | <0.001 |
| Interleukin-3 | 1.07 ± 0.02 | 1.05 ± 0.06 | 0.37 |
| Interleukin-4 | 1.09 ± 0.09 | 0.90 ± 0.12 | 0.10 |
| Interleukin-5 | 7.86 ± 1.14 | 0.29 ± 0.29 | <0.001 |
| Interleukin-7 | 1.75 ± 0.18 | 2.14 ± 1.16 | 0.37 |
| Interleukin-9 | 311.8 ± 38.17 | 79.57 ± 15.47 | <0.001 |
| Interleukin-12 (p40) | 7.13 ± 1.89 | 3.27 ± 1.04 | 0.049 |
| Interleukin-12 (p70) | 1.95 ± 1.00 | 2.88 ± 0.93 | 0.19 |
| Interleukin-13 | 10.56 ± 1.49 | 7.92 ± 1.92 | 0.15 |
| Interleukin-15 | 6.10 ± 1.02 | 1.85 ± 0.57 | 0.002 |
| Interleukin-17 | 2.28 ± 0.56 | 12.34 ± 4.67 | 0.03 |
| Eotaxin | 4.63 ± 2.43 | 21.59± | 0.09 |
| Interferon-γ | 8.08 ± 2.18 | 103.9 ± 92.04 | 0.16 |
| Granulocyte-Macrophage Colony- | 0.00 ± 0.00 | 0.38 ± 0.38 | 0.17 |
| Stimulating Factor | | | |
| Interferon-γ-Induced Protein-10 | 118.40 ± 43.72 | 616.90 ± 171.1 | 0.01 |
| Keratinocyte Chemoattractant/Growth | 19.54 ± 3.08 | 35.89 ± 12.54 | 0.11 |
| Regulated Oncogene | | | |
| Monocyte Chemoattractant Protein | 14.66 ± 7.16 | 34.20 ± 15.08 | 0.13 |
| Macrophage Inflammatory Protein-1α | 45.99 ± 7.56 | 73.96 ± 15.40 | 0.07 |
| Macrophage Inflammatory Protein-1β | 19.42 ± 7.63 | 70.89 ± 21.44 | 0.02 |
| Macrophage Inflammatory Protein-2 | 28.63 ± 5.43 | 16.54 ± 7.99 | 0.11 |
| Monocyte Induced by Interferon-γ | 100.60 ± 35.47 | 906.00 ± 295.40 | 0.01 |
| Macrophage Colony-Stimulating Factor | 2.89 ± 1.02 | 1.02 ± 0.41 | 0.049 |
| C-X-C Motif Chemokine 5 (LIX) | 0.00 ± 0.00 | 0.00 ± 0.00 | 0.99 |
| Vascular Endothelial Growth Factor | 12.18 ± 3.03 | 12.64 ± 5.22 | 0.47 |

### DISCUSSION

In this Example, we provide results demonstrating that F-652 treatment led to decreased inflammation in the lungs as demonstrated by less immune cellular influx (FIG. 13) in a mouse model of ALI/ARDS. F-652 reduced expression of inflammatory cytokines in the lung, including Interleukin-6 and TNF-α. Both of these inflammatory mediators were found to be decreased in F-652 treated mice after LPS injury (FIG. 14). Our findings are consistent with previous studies that have showed decreased total cell counts, neutrophils, lymphocytes, and macrophages in the BAL of mice on a pro-IL-22 genetic setting after influenza injury.

Also provided in this Example are results demonstrating that treatment with F-652 decreased protein leak and helped maintain the endothelial glycocalyx (EGX) after low-dose LPS injury (FIG. 16). Degradation of the glycocalyx has been implicated in the fluid and protein leak that occurs in ARDS and protection of the glycocalyx after lung injury mitigates the changes seen in the lung during ARDS (Murphy, L. S., et al., “Endothelial glycocalyx degradation is more severe in patients with non-pulmonary sepsis compared to pulmonary sepsis and associates with risk of ARDS and other organ dysfunction.” Annals of Intensive Care, 2017. 7(1): p. 1-9; Kong, G., et al., “Astilbin alleviates LPS-induced ARDS by suppressing MAPK signaling pathway and protecting pulmonary endothelial glycocalyx.” Int Immunopharmacol, 2016. 36: p. 51-58; Wang, L., et al., “Ulinastatin attenuates pulmonary endothelial glycocalyx damage and inhibits endothelial heparanase activity in LPS-induced ARDS.” Biochem Biophys Res Commun, 2016. 478(2): p. 669-75). Preservation of the glycocalyx can occur by suppression of metalloproteinases or heparinases or by induction of the biosynthesis of the glycoprotein layer, as demonstrated in Example 3 above.

RNA-seq demonstrated decreased expression of CCL4. This was confirmed with decreased CCL4 in BAL for high-dose LPS injured mice treated with F-652. CCL4 has a strong inflammatory and chemotactic effect, and anti-inflammatory effects seen with F-652 treatment may in part be due to its decreased CCL4 expression. RNA-seq also demonstrated increased expression of several extracellular matrix-receptor interactions, including Tenascin C (Tnc), collagen, type I, alpha 1 (COL1a1), collagen, type VI, alpha 3 (Col6a3), and collagen, type I, alpha 2 (Col1a2) expression. Collagen, type I, alpha 1 and type I, alpha 2 are important extracellular matrix components in the repair process of the lung after acute lung injury (de Souza Xavier Costa, N., et al., “Early and late pulmonary effects of nebulized LPS in mice: An acute lung injury model.” PLoS One, 2017. 12(9): p. e0185474). The prevalence of these gene products in the presence of a decrease in inflammatory mediators seen in the F-652 treated animals suggests that the injured lungs have moved on from an inflammatory stage to a reparative stage.

In conclusion, F-652 leads to decreased inflammation (FIG. 13) and protein leak in a pre-clinical model of ALI. F-652 preserves the EGX (FIG. 16) and leads to increased endogenous IL-22 production (FIG. 17). These findings suggest a potential therapeutic effect of F-652 in virus-induced lung injury or failure (e.g., ALI/ARDS).

### Example 5. Randomized, Double-Blind, Placebo-Controlled, Dose-Escalation, Multicenter Study to Evaluate the Efficacy and Safety of F-652 in Patients with Moderate to Severe COVID-19

### Study Description

The primary objective of this study is to evaluate the safety and efficacy of F-652 when intravenously (IV) administered in hospitalized, confirmed COVID-19 adult patients with moderate to severe symptoms. The secondary objective is to evaluate the pharmacodynamics (PD) of F-652 when IV administered in hospitalized, confirmed COVID-19 adult patients with moderate to severe symptoms.

Study Design and Duration

This is an interventional, multicenter, 2-arm, parallel-group, randomized, double-blind, placebo-controlled, dose-escalation, safety and efficacy study of F-652 treatment versus placebo in patients aged 18 years or older with a COVID-19 diagnosis confirmed by PCR. Eligible patients will have moderate to severe COVID-19 symptoms within 5 days post hospitalization and a positive COVID-19 testing.

The study is planned to include 4 cohorts, with enrolled patients being randomized 1:1 in a blinded manner on Day 1, following screening, to F-652 or placebo as follows:

Cohort 1 (sentinel cohort): Four patients will receive either 30 μg/kg F-652 or placebo. Two patients will receive F-652 and 2 patients will receive placebo. Upon completion of sentinel dosing (7 days after last patient last dose), the Data Monitoring Committee (DMC) will evaluate the safety and tolerability data of the sentinel patients and determine if it is acceptable to dose the remaining patients in this dosing group in Cohort 2.

Cohort 2: Fourteen patients will receive either 30 μg/kg F-652 or placebo. Seven patients will receive F-652 and 7 patients will receive placebo. Upon completion of Cohort 2, the DMC will convene and review all available safety data to determine if the study can proceed to the next dose level.

Cohort 3 (sentinel cohort): Four patients will receive either 45 μg/kg F-652 or placebo. Two patients will receive F-652 and 2 patients will receive placebo. Upon completion of sentinel dosing (7 days after last patient last dose), the DMC will evaluate the safety and tolerability data of the sentinel patients and determine if it is acceptable to dose the remaining patients in this dosing group in Cohort 4.

Cohort 4: Sixteen patients will receive either 45 μg/kg F-652 or placebo. Eight patients will receive F-652 and 8 patients will receive placebo.

Treatment will begin on Day 1 following randomization. Patients assigned to active drug will receive a total of 2 doses of F-652 (1 IV infusion on Day 1 and 1 IV infusion on Day 8). Patients assigned to placebo will receive identical IV infusions of placebo vehicle on Days 1 and 8. All patients will receive available supportive and antiviral therapies as standard of care. Efficacy will be assessed on Days 15 and 29. Patients will be followed for safety until Day 60.

Dosage Forms and Route of Administration

F-652 is a recombinant fusion protein consisting of human IL-22 and human immunoglobulin G2 Fc fragments. F-652 is produced in Chinese Hamster Ovary cells, with an immunoglobulin-like structure with 2 IL-22 molecules (recombinant human IL-22 dimer) at the N-terminal. F-652 will be administered, based on the patient's most recent weight, at a dose of 30 μg/kg or 45 μg/kg IV on Days 1 and 8. Placebo vehicle will be identical in appearance to the study drug and will be administered IV on Days 1 and 8.

Efficacy Endpoints

Primary Efficacy Endpoints

The primary efficacy endpoint is the proportion of patients with a greater than or equal to 2-point increase in the National Institute of Allergy and Infectious Diseases (NIAID) 8-point ordinal scale from baseline to Day 29.

The NIAID 8-point ordinal scale includes the following grades: 1. Death; 2. Hospitalized, on invasive mechanical ventilation or extracorporeal membrane oxygenation; 3. Hospitalized, on non-invasive ventilation or high-flow oxygen devices; 4. Hospitalized, requiring supplemental oxygen; 5. Hospitalized, not requiring supplementation oxygen—requiring ongoing medical care (COVID-19 related or otherwise); 6. Hospitalized, not requiring supplemental oxygen—no longer requires ongoing medical care; 7. Not hospitalized, limitation on activities and/or requiring home oxygen; and 8. Not hospitalized, no limitations on activities.

Secondary Efficacy Endpoints

The secondary efficacy endpoints, listed in hierarchical order, include the following: (a) Length of hospital stay from first dosing (Day 1) and percentage of patients who have recovered and discharged from the hospital by Days 15 and 29; (b) Mortality rate by Days 15 and 29; (c) Proportion of patients with a ≥2-point increase in the NIAID 8-point ordinal scale from baseline to Day 15; (d) Alive and respiratory failure free days by Days 15 and 29; (e) Percentage of patients progressed to severe/critical disease by Day 15; and (f) Occurrence of any new infections during the study by Day 29.

Safety Endpoints

The safety endpoints include the following: (a) All cause treatment-emergent adverse events (TEAEs) and serious adverse events (SAEs); (b) Change from screening (baseline) in clinical symptoms and abnormal vital signs, abnormal laboratory tests (e.g., complete blood count, serum chemistry, routine urinalysis, and coagulation function), and 12-lead electrocardiograms (ECGs); and (c) Relationship of any observed adverse events (AEs) with F-652 treatment based on the Investigator's judgement.

Exploratory Endpoints

The exploratory endpoints include the following: (a) Time to negative SARS-CoV-2 PCR test from randomization; and (b) Changes in PD parameters, including serum amyloid A (SAA), C-reactive protein (CRP), regenerating islet-derived 3 (Reg3), IL-6, IL-17, TNF-α, ferritin, and troponin-I.

### Example 6. Study of Therapeutic Effects of F-652 Against COVID-19 in Primary Human Bronchial Epithelial Cells

Provided in this Example are results demonstrating that F-652 (IL-22-Fc fusion protein) alleviates SARS-CoV-2 infection in primary human bronchial epithelial (HBE) cells.

Primary HBE cells were cultured in a 24-well transwell plate at air-liquid interface. They were either pre-treated with F-652 before SARS-CoV-2 infection, or post-treated with F-652 after SARS-CoV-2 infection. For the pre-treatment condition, 100 ng/mL F-652 in 300 μL medium was added to the cultured HBE cells for 18 hours at 37° C., 5% CO₂ overnight. For the post-treatment condition, 100 ng/mL of F-652 in 300 μL medium was added basolaterally on the day post-viral infection. No F-652 treatment post-infection, and non-infected HBE cells served as controls. SARS-CoV-2 infection of HBE cells was performed by adding 20 μL of virus stock [10⁵ pfu] (MOI of 0.1; or 100,000 pfu per well) to the apical surface of the cultured HBE cells. The plates were incubated for 2 hours to allow viral attachment at 37° C., 5% CO₂, and the viral suspension was then removed from each well. 48 hours post challenge, HBE cells were transferred into a new 24-well transwell plate, and total RNAs was harvested by lysing the cells in 300 μL Trizol per well, following the Direct-zol™ RNA kit instruction. Viral load was assayed with subgenomic-N (sgm-N) RNA standard, as subgenomic RNA measures new viral RNA, not just the viral inoculum. RNA-seq was also conducted, followed by mapping of the reads to determine the read counts per SARS-CoV-2 open reading frame (ORF).

As assayed by subgenomic RNA, both pre-treatment and post-treatment with F-652 showed significantly lower copies of sgm-N RNA copies compared to no F-652 treatment group (p<0.05, ANOVA, Tukey's multiple comparisons test; FIG. 18A), which was also consistent with reduced mapping of RNA-seq reads to the SARS-CoV-2 genome compared to the no F-652 treatment group (FIG. 18B). These results demonstrate both preventive and therapeutic effects of F-652 against COVID-19.

### Example 7. Study of Therapeutic Effects of F-652 in Age-Related Viral Pneumonia

Provided in this Example are results demonstrating that F-652 (IL-22-Fc fusion protein) is particularly effective for treating viral (e.g., H1N1 influenza) pneumonia and ameliorating chronic lung fibrosis induced by vrial infection in aged hosts.

Studies have shown that vast majority of severe COVID-19 cases occur in the elderly population (A. Remuzzi and G. Remuzzi Lancet, 2020, VOL. 395, Issue 10231, P1225-1228). Emerging evidence has suggested that COVID-19 survivors exhibit persistent impairment of lung function due to the development of lung fibrosis (YH. Xu et al. J Infect. 2020; 80(4):394-400; S. Zhou et al. AIR Am J Roentgenol. 2020; 214(6):1287-1294; M. Hosseiny et al. AJR Am J Roentgenol. 2020; 214(5):1078-1082). Lung fibrosis was also documented in a substantial number of patients who have recovered from the infection of SARS-CoV or MERS-CoV (K. S. Chan et al. Respirology. 2003; 8 Suppl(Suppl 1):536-40; G. E. Antonio et al. Radiology. 2003; 228(3):810-815), two closely related coronavirus of SARS-CoV-2. It is estimated that there will be a large number of individuals who recover from COVID-19 to develop chronic lung fibrosis. However, there are no preventive means nor therapeutic interventions available to slow down and/or reverse lung fibrosis development following any viral pneumonia, especially COVID-19.

Influenza pneumonia is known to lead to persistent lung collagen deposition (reflection of fibrosis; Z. Wang et al. Sci Immunol. 2019; 4(36):eaaw1217; S. Huang et al. PLoS One. 2019; 14(10):e0223430), and was used herein as an exemplary disease model of lung fibrosis following viral pneumonia, providing insight for COVID-19 treatment.

### Study Design

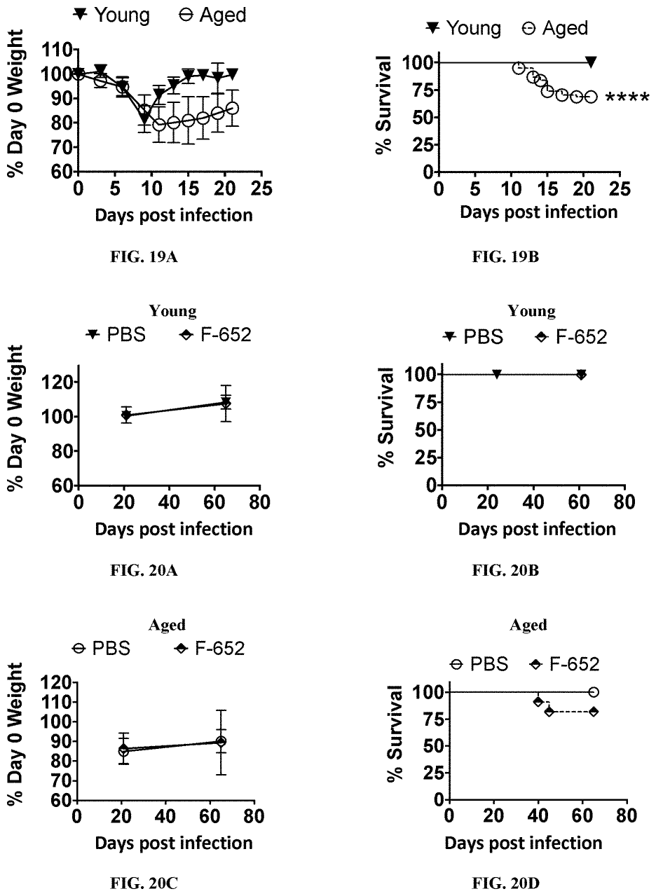

Aged (18-19 month old C57BL/6 mice from Jackson laboratory) and young mice (2 month old C57BL/6J) were infected with H1N1 influenza (A/PR8 strain) on Day 0. They were weighed on alternating days Day 21 post-viral infection. All animals that dropped <10% of Day 0 body weight during Days 0-21 post-infection were excluded from further study, and the remaining animals were weighed to obtain an average weight for young and age groups, separately. As can be seen from FIGS. 19A-19B, this H1N1 influenza infection model was a severe age-related model in terms of both morbidity and mortality, in which aged infected mice experienced more weight loss and significantly more death incidences compared to young infected mice.

At Day 21 post-infection, 61 aged mice and 40 young mice were randomized into 4 groups: (i) young infected mice treated with 200 μL PBS intravenously on tail vein; (ii) young infected mice treated with 200 μg/kg F-652 in 200 μL intravenously on tail vein; (iii) aged infected mice treated with 200 μL PBS intravenously on tail vein; and (iv) aged infected mice treated with 200 μg/kg F-652 in 200 μL intravenously on tail vein. One week post-injection of PBS or F-652, tails of the aged animals had not recovered from intravenous injection, so remaining treatments were intraperitoneal injections (dose/volume unchanged). The four study groups received PBS or F-652 injections for 3 weeks, 1 treatment/week/mouse, starting from Day 21 post-viral infection. A similar set of experiments were conducted on age- and treatment matched cohorts (4 groups) but treated with either PBS or F-652 for 6 weeks, 1 treatment/week/mouse (hereinafter referred to as “6-week treatment group”; data not shown). Unless indicated otherwise, all data presented in this Example are 3-week treatment data.

At the end-point Day 62-65 post-infection, animals were intravenously injected with anti-CD45 antibody to distinguish between circulating leukocytes (CD45+) and lung parenchymal cells by flow cytometry, prior to measurements of lung function, lung histopathology, lung immune profiles, and lung collagen content.

Lung function was measured under tidal breathing conditions explained in detail in Goplen et al. (J Allergy Clin Immunol. 2009; 123(4): 925-32.el 1). Various perturbations were performed before and following deep inflation which recruits closed airways. These measurements were compared to pre-inflation data to determine baseline vs. lung capacity lung physiology for single compartment, constant phase, and pressure volume loops on a flexiVent® (Scireq) computer controlled piston respirator. See FIG. 24 for exemplary experimental set up.

### Treatment Results Prior to End-Point

Prior to treatment, no young mice succumb to viral infection (FIGS. 19B and 20B), whereas ˜25% of aged mice met IACUC cutoffs or were found dead prior to losing >30% of Day 0 body weight. During the treatment phase, no mice were lost in the young groups (FIG. 20B), but 3 mice were lost between Days 21-64 post-infection in the aged F-652 treatment group (FIG. 20D; 2 mice were lost in the aged F-652 6-week treatment group, data not shown), no PBS treated mice were lost (ANOVA p>0.05). During the same time period, no notable differences in weight occurred between PBS and F-652 treatment groups either in young (FIG. 20A) or aged (FIG. 20C) mice. These results demonstrated that F-652 treatment had no or little adverse effect on body weight or survival in either young or aged mice infected with H1N1.

### End-Point Results

Flow Cytometry

Prior to sacrifice, circulating white-blood cells were labeled intravenously with anti-CD45 antibody. All animals were sacrificed on Days 62-65 post-infection in age- and treatment-matched cohorts (4 groups). Lung tissues were harvested. Tissue-infiltrating myeloid cell number in combined right lung lobes were studied from each group. Following lung tissue digestion, multi-parameter FACS was used to separate circulating white-blood cells (CD45+) from lung parenchymal cells, and distinguishing between tissue-infiltrating neutrophils (CD11b^{Hi} Ly6G^{Hi}) or inflammatory monocytes (CD11b^{Hi} Ly6C^{Hi}), and tissue-infiltrating CD8+ T cells.

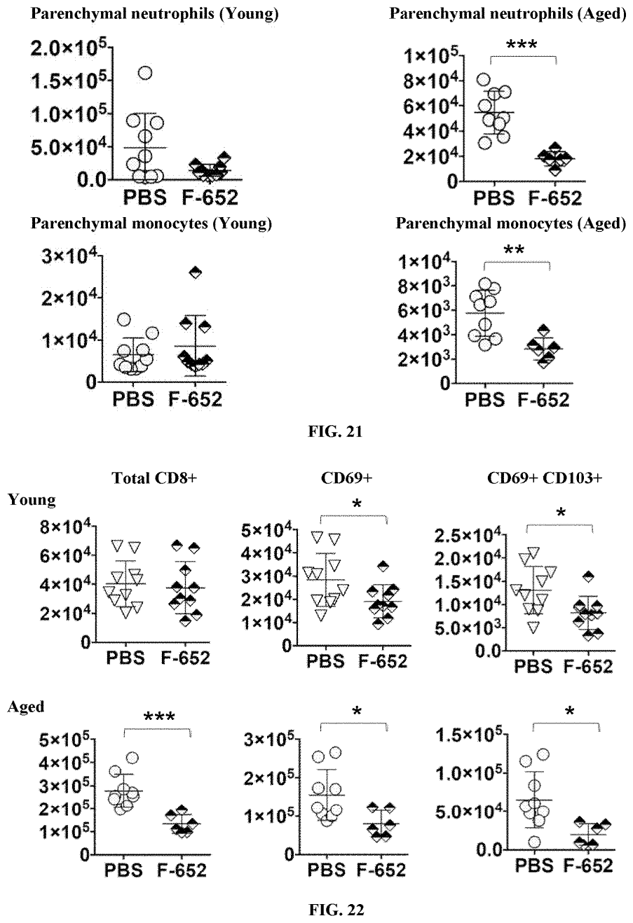

As can be seen from FIG. 21, both lung infiltrating neutrophils and inflammatory monocytes decreased significantly in F-652 treated aged mice (compared to PBS control). 6-week F-652 treatment resulted in even more decrease in lung infiltrating neutrophils and inflammatory monocytes in aged mice, compared to the 3-week F-652 treatment aged group (data not shown). However, no significant difference was observed between PBS and F-652 treatments in young mice for either lung infiltrating neutrophils or inflammatory monocytes, either treated for 3 weeks (FIG. 21) or 6 weeks (data not shown).

Similarly, influenza specific CD8+ T cells, which were found not to be protective, but pathogenic in aged animals, significantly decreased in F-652 treated aged mice compared to PBS control; but no significant difference was observed in young mice for total number of CD8_T cells (see FIG. 22 “Total CD8+”). This pattern was consistent with that seen for infiltrating neutrophils and monocytes. CD8+ T cells expressing CD69+ or CD69+/CD103+ decreased significantly in F-652 treatment group compared to PBS control, in both young and aged hosts.

These results demonstrate that i) F-652 treatment significantly dampens exacerbated monocyte and neutrophil infiltration in the lung of aged H1N1 hosts; and ii) F-652 treatment significantly dampens resident-like CD8+ T cells in both young and aged H1N1 hosts, but especially in aged hosts where CD8+ T cells have been shown to be pathogenic.

Lung Function

Lung function studies were conducted on mice Days 63-67 post-infection. As can be seen from FIG. 24, tracheotomy was performed with a 19G cannula and connected to the flexiVent® via Y-tubing. A computer controlled piston delivered a pre-determined volume and frequency of air over time. The air pressure was measured before going into and after coming out of the lungs, and pressure-volume data was fit to various lung models. flexiVent® was used to measure the whole respiratory system, conduct compartmental analysis, and take both baseline and total capacity measurements.

In a broadband forced oscillation manoeuvre (a.k.a. low-frequency forced oscillation technique (FOT)) the subject's response to a signal containing a wide range of frequencies both below and above the subject's breathing frequency is measured. The outcome, respiratory system input impedance (Zrs), is the most detailed assessment of respiratory mechanics currently available. Input impedance can be further analyzed using the Constant Phase Model (CPM), to obtain a parametric distinction between airway and tissue mechanics, providing insights on how diseases affect lungs. Input Impedance (Zrs) is the combined effects of resistance, compliance and inertance as a function of frequency. Resistance (R; dynamic resistance) quantitatively assesses the level of constriction in the lungs. Compliance (C; also known as dynamic compliance) describes the ease with which the respiratory system can be extended. In a subject with intact chest walls, it provides a characterization of the overall elastic properties that the respiratory system needs to overcome during tidal breathing to move air in and out of the lungs. Tissue damping (G) is a parameter of the CPM closely related to tissue resistance and reflects the energy dissipation in the alveoli.

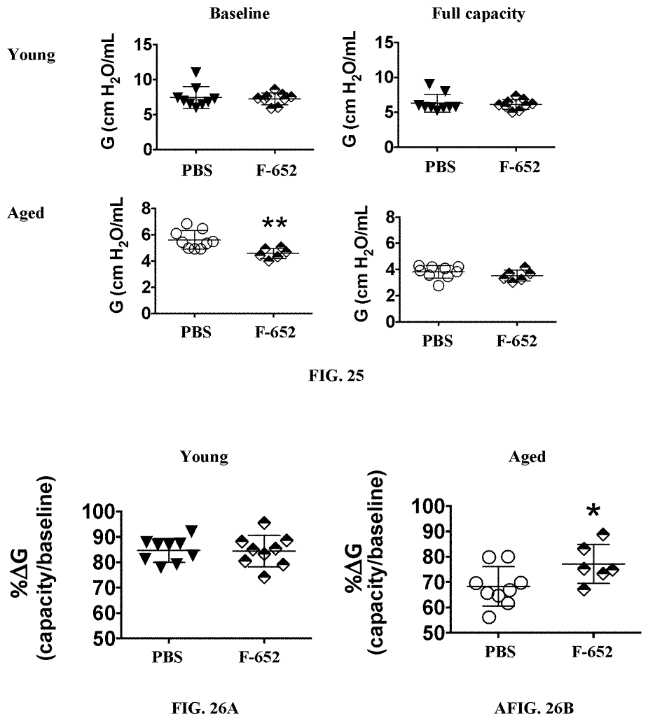

Tissue dampening (G) was measured by FOT in young (FIG. 25 top panels) and aged (FIG. 25 bottom panels) mice treated (F-652) or not treated (PBS) prior to (“baseline” panels) and following (“full capacity” panels) airway recruitment maneuver. These measurements were then normalized (capacity G/baseline G reflected as “% ΔG”) to determine % tissue dampening (airway resistance in parenchyma), see FIGS. 26A-26B. As can be seen from FIGS. 25-26B, F-652 treatment led to less resistance in small airways in aged H1N1 infected mice during baseline/tidal breathing. These data demonstrate that F-652 improves baseline function of lung parenchyma by decreasing resistance to airflow following H1N1 infection in aged, but not young mice.

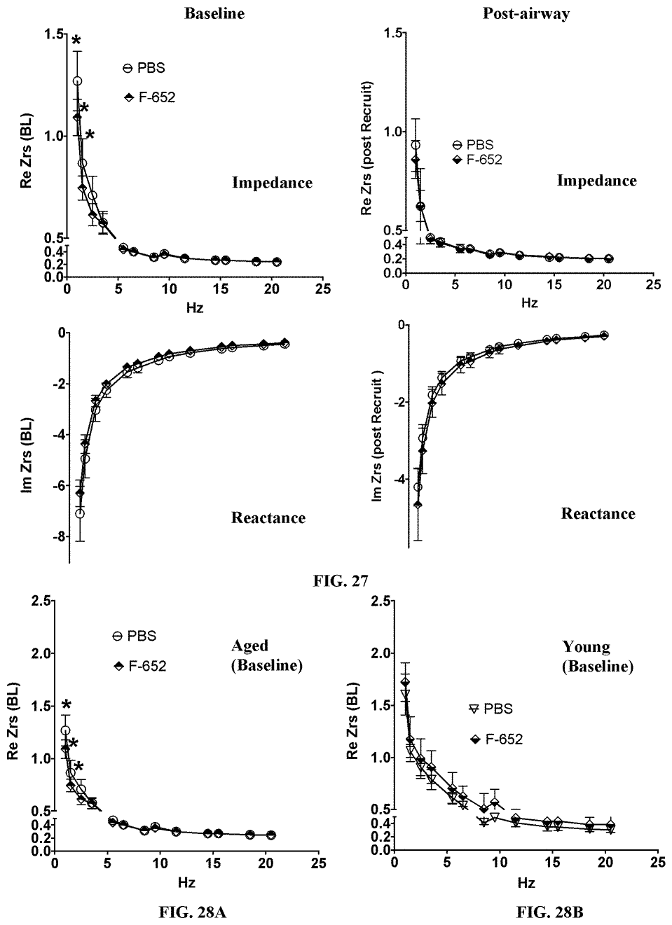

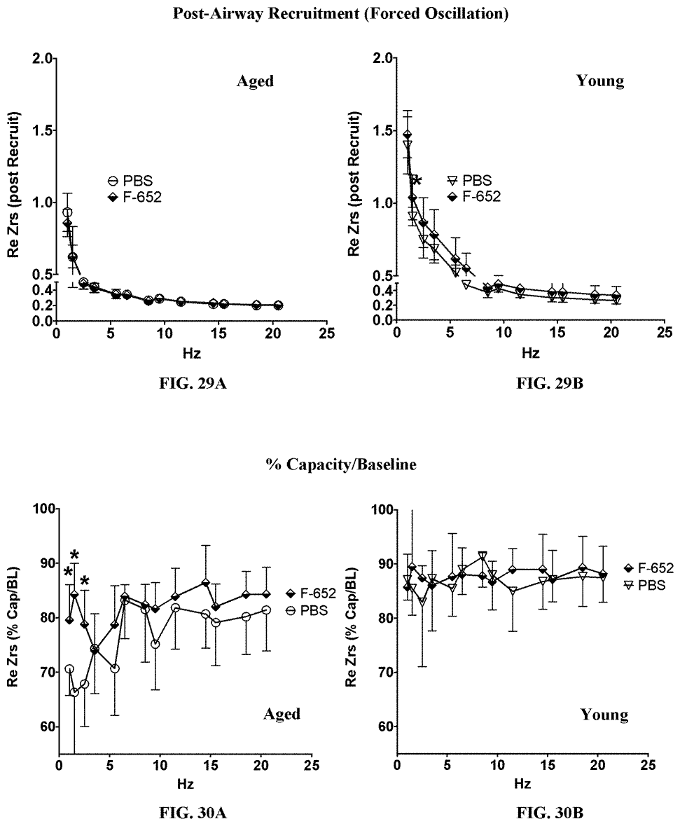

Input impedance (Re Zrs) and reactance (Im Zrs; FIG. 27 bottom panels) were measured with FOT on the flexiVent® prior to (“baseline”) and following (“post-airway”) airway recruitment maneuver in young (FIGS. 27, 28A, 29A) or aged (FIGS. 27, 28B, 29B) mice treated (F-652) or not treated (PBS). Input impedance (Re Zrs) data were then normalized at each frequency, as reflected by % Re Zrs (capacity Re Zrs/baseline Re Zrs) for aged (FIG. 30A) and young (FIG. 30B) mice treated (F-652) or not treated (PBS). These data demonstrate that i) F-652 treatment significantly improves baseline resistance (lowers baseline airflow resistance) in small airways of aged mice, not young mice; and ii) F-652 treatment has no effect on impedance following maximization of available lung volume (FIGS. 29A-29B).

As can be seen from FIGS. 25-30B, the Constant Phase Model (CPM), which separates large and small airway measurements for resistance to airflow, indicated that in the 3-week treatment groups, aged (see “aged baseline” panel in FIG. 25, FIG. 26B, FIG. 27, FIG. 28A, FIG. 30A) but not young (compare old vs. young in FIGS. 25, 26A, 26B, and 28A-30B) F-652 treatment groups showed decreased resistance in the small airways at baseline (compare “baseline impedence” and “post-airway impedence” panels in FIG. 27), indicating that aged F-652 treated mice used a higher percentage of their small airways than matched PBS controls. This pattern differences were not seen in the 6-week treatment groups.

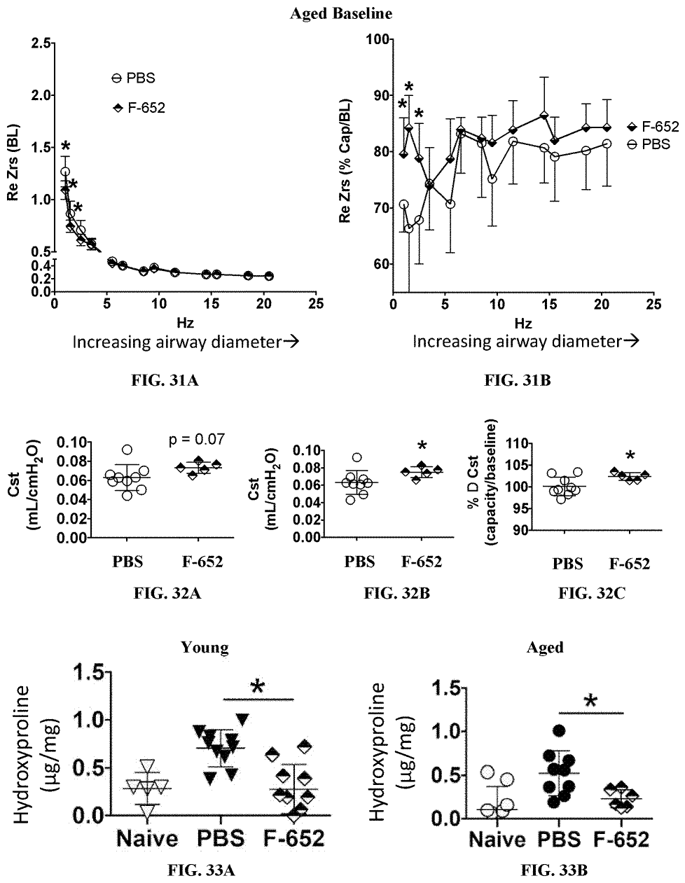

More in depth analysis of CPM probed by FOT (input impedence measurements) of the respiratory system showed that the improvements in lung function in the aged 3 week treatment group at baseline (tidal breathing) was a result of differences in the smallest diameter airways, most likely indicating improvement of alveolar use. See FIGS. 31A-31B “*” indicated portions, indicating that F-652 treatment lowers baseline airflow resistance in small airways in aged animals.

All these data demonstrate that F-652 improves age-related dysfunction of small airways during tidal breathing (baseline), which could prevent airway collapse and increase compliance.

Pressure-volume (PV) loops capture the quasi-static mechanical properties of the respiratory system. Cst (quasi-static compliance) is a classic parameter extracted from a PV curve. If measured under closed-chest conditions, it reflects the intrinsic elastic properties of the respiratory system (i.e. lung+chest wall) at rest. Static compliance was determined in aged mice treated with F-652 or PBS control from PV loop maneuvers during tidal breathing (FIG. 32A), post-airway recruitment (FIG. 32B), and normalized to each other (FIG. 32C). As can be seen from FIGS. 32A-32C, PV loops indicated an increased static compliance in aged F-652 treatment group relative to PBS controls. These data demonstrate that F-652 treatment decreases the stiffness of the lung (increases compliance), indicating improved breathing at baseline, and that the physical properties governing lung elasticity and rigidity are changed by F-652 treatment.

Right lung lobes were minced and mixed to homogeneity from different groups. A 30-40 mg sample was taken from each lung prep and determined for hydroxyproline content, which is the major component of collagen. As can be seen from FIGS. 33A-33B, F-652 treatment significantly reduced hydroxyproline content to similar level from non-infected lung tissues (“naïve”), compared to PBS control, in both aged and young H1N1-infected mice, indicating that F-652 treatment can lower H1N1-induced collagen deposition. These data demonstrate a likely improvement in post-pneumonia fibrosis (reducing fibrosis) from F-652 treatment, which is consistent with increased static compliance seen from PV loop study.

The improved lung function following F-652 treatment was likely due to one or more of: i) decreased collagen content and/or increased elastin content; ii) increased Type I/II pneumocyte (surface epithelial cells of the alveoli) generation; and iii) increased surfactant.

Histology

Paraffin-embedded lungs from aged H1N1-infected mice were sliced and stained with hematoxylin and eosin (H&E), Masson's Trichrome, Sirius Red, or Periodic acid-Schiff (PAS), then images were taken on an Aperio scanner with 40×resolution. Non-infected healthy lung tissue served as negative control. In H&E staining, hematoxylin stains cell nuclei blue, and eosin stains the extracellular matrix and cytoplasm pink. Masson's trichrome stains collagen blue or green. Collagen fibers are stained red in Sirius Red staining. PAS staining produces a purple-magenta color for glycogen, glycoproteins, or glycolipids.

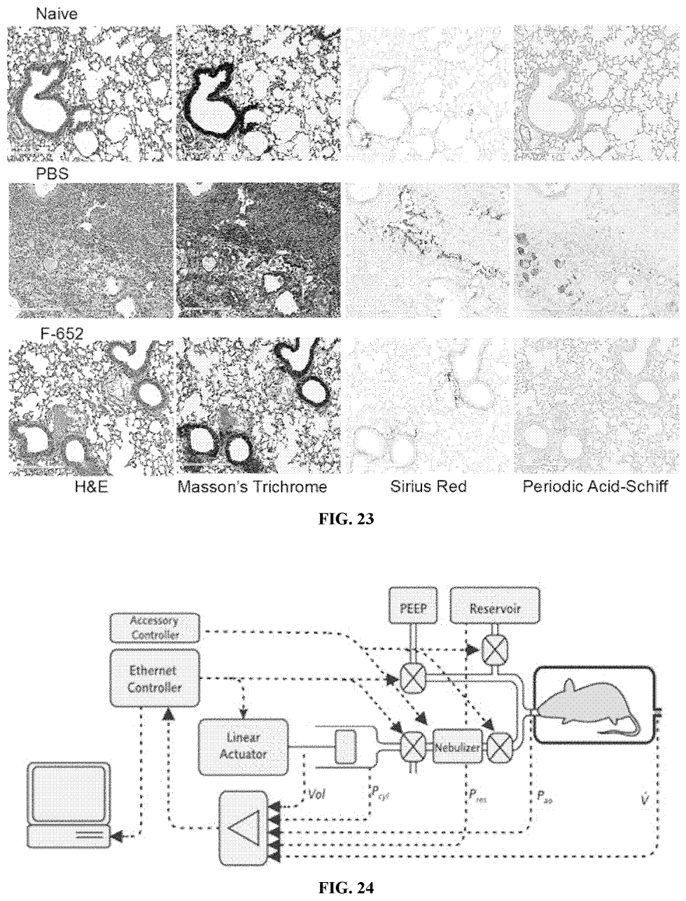

As can be seen from FIG. 23, F-652 treatment ameliorates much of the H1N1-induced pathology in aged hosts. Lung histology largely matched lung function and FACS data, indicating that the one group that benefited greatly from F-652 treatment was the aged group treated for 3 weeks. The lack of neutrophil and monocyte infiltration and CD8+ T cells seen by FACS can be clearly seen in these histological samples which correlated nicely with improved lung function.

Lung Damage Repair

Keratin 5 (KRT5) dimerizes with keratin 14 and forms the intermediate filaments that make up the cytoskeleton of basal epithelial cells. KRT5+ cells in the lung indicate stem cells that have not fully differentiated into pneumocytes. Immunofluorescence staining of lungs harvested from aged mice with anti-CD8 and anti-KRT5 antibodies showed a clear trend of decrease of CD8+ cells and KRT5+ cells in F-652 treated lungs compared to PBS control (data not shown). These results indicate an improvement of lung repair following viral pneumonia in aged hosts treated with F-652, which showed increased lung function and decreased immune cell infiltrates.

To summarize, these data demonstrate that F-652 is particularly effective for treating Influenza (e.g., H1N1)-induced pneumonia and improving lung functions in aged hosts, like by ameliorating lung fibrosis, improving lung repair, and reducing immune cell infiltration. It shed light on F-562's therapeutic effects in the treatment of chronic pulmonary fibrosis caused by COVID-19 pneumonia, which mainly occurs in the aged population. See mouse vs. human age in C. Hagan, November 2017, Blog Post from the Jackson Laboratory.

SEQUENCE LISTING
(linker)
SEQ ID NO: 1
GSGGGSGGGGSGGGGS
(linker)
SEQ ID NO: 2
GGSGGS
(linker)
SEQ ID NO: 3
SGGGGS
(linker)
SEQ ID NO: 4
GRAGGGGAGGGG
(linker)
SEQ ID NO: 5
GRAGGG
(linker; n is an integer of at least 1)
SEQ ID NO: 6
(G)ₙ
(linker; n is an integer of at least 1)
SEQ ID NO: 7
(GS)ₙ
(linker; n is an integer of at least 1)
SEQ ID NO: 8
(GSGGS)ₙ
(linker; n is an integer of at least 1)
SEQ ID NO: 9
(GGGS)ₙ
(linker)
SEQ ID NO: 10
ASTKGP
(linker; n is an integer of at least 1)
SEQ ID NO: 11
(GGGGS)ₙ
(linker)
SEQ ID NO: 12
GG
(linker)
SEQ ID NO: 13
GGSG
(linker)
SEQ ID NO: 14
GGSGG
(linker)
SEQ ID NO: 15
GSGSG
(linker)
SEQ ID NO: 16
GSGGG
(linker)
SEQ ID NO: 17
GGGSG
(linker)
SEQ ID NO: 18
GSSSG
(linker)
SEQ ID NO: 19
GGGGSGGGGSGGGGS
(linker)
SEQ ID NO: 20
GGGGS
(human IL-22 (mature))
SEQ ID NO: 21
APISSHCRLDKSNFQQPYITNRTFMLAKEASLADNNTDVRLIGEKLFHGVSMSERCYLMKQVLNF
TLEEVLFPQSDRFQPYMQEVVPFLARLSNRLSTCHIEGDDLHIQRNVQKLKDTVKKLGESGEIKAI
GELDLLFMSLRNACI
(human IgG2 Fc (P107S))
SEQ ID NO: 22
VECPPCPAPPVAGPSVFLFPPKPKDTLMISRTPEVTCVVVDVSHEDPEVQFNWYVDGVEVHNAK
TKPREEQFNSTFRVVSVLTVVHQDWLNGKEYKCKVSNKGLPASIEKTISKTKGQPREPQVYTLPP
SREEMTKNQVSLTCLVKGFYPSDIAVEWESNGQPENNYKTTPPMLDSDGSFFLYSKLTVDKSRW
QQGNVFSCSVMHEALHNHYTQKSLSLSPGK
(human IgG2 Fc)
SEQ ID NO: 23
ERKCCVECPPCPAPPVAGPSVFLFPPKPKDTLMISRTPEVTCVVVDVSHEDPEVQFNWYVDGVE
VHNAKTKPREEQFNSTFRVVSVLTVVHQDWLNGKEYKCKVSNKGLPAPIEKTISKTKGQPREPQ
VYTLPPSREEMTKNQVSLTCLVKGFYPSDISVEWESNGQPENNYKTTPPMLDSDGSFFLYSKLTV
DKSRWQQGNVFSCSVMHEALHNHYTQKSLSLSPGK
(F-652; IL-22-linker-IgG2 Fc (P107S); linker is bolded)
SEQ ID NO: 24
APISSHCRLDKSNFQQPYITNRTFMLAKEASLADNNTDVRLIGEKLFHGVSMSERCYLMKQVLNF
TLEEVLFPQSDRFQPYMQEVVPFLARLSNRLSTCHIEGDDLHIQRNVQKLKDTVKKLGESGEIKAI
GELDLLFMSLRNACIGSGGGSGGGGSGGGGSVECPPCPAPPVAGPSVFLFPPKPKDTLMISRTPE
VTCVVVDVSHEDPEVQFNWYVDGVEVHNAKTKPREEQFNSTFRVVSVLTVVHQDWLNGKEYK
CKVSNKGLPASIEKTISKTKGQPREPQVYTLPPSREEMTKNQVSLTCLVKGFYPSDIAVEWESNG
QPENNYKTTPPMLDSDGSFFLYSKLTVDKSRWQQGNVFSCSVMHEALHNHYTQKSLSLSPGK
(IgG2 Fc (P107S)-linker-IL-22; linker is bolded)
SEQ ID NO: 25
VECPPCPAPPVAGPSVFLFPPKPKDTLMISRTPEVTCVVVDVSHEDPEVQFNWYVDGVEVHNAK
TKPREEQFNSTFRVVSVLTVVHQDWLNGKEYKCKVSNKGLPASIEKTISKTKGQPREPQVYTLPP
SREEMTKNQVSLTCLVKGFYPSDIAVEWESNGQPENNYKTTPPMLDSDGSFFLYSKLTVDKSRW
QQGNVFSCSVMHEALHNHYTQKSLSLSPGKGSGGGSGGGGSGGGGSAPISSHCRLDKSNFQQP
YITNRTFMLAKEASLADNNTDVRLIGEKLFHGVSMSERCYLMKQVLNFTLEEVLFPQSDRFQPY
MQEVVPFLARLSNRLSTCHIEGDDLHIQRNVQKLKDTVKKLGESGEIKAIGELDLLFMSLRNACI
(IL-22-linker-IgG2 Fc (P107S); linker is bolded)
SEQ ID NO: 26
APISSHCRLDKSNFQQPYITNRTFMLAKEASLADNNTDVRLIGEKLFHGVSMSERCYLMKQVLNF
TLEEVLFPQSDRFQPYMQEVVPFLARLSNRLSTCHIEGDDLHIQRNVQKLKDTVKKLGESGEIKAI
GELDLLFMSLRNACIASTKGPVECPPCPAPPVAGPSVFLFPPKPKDTLMISRTPEVTCVVVDVSHE
DPEVQFNWYVDGVEVHNAKTKPREEQFNSTFRVVSVLTVVHQDWLNGKEYKCKVSNKGLPASI
EKTISKTKGQPREPQVYTLPPSREEMTKNQVSLTCLVKGFYPSDIAVEWESNGQPENNYKTTPPM
LDSDGSFFLYSKLTVDKSRWQQGNVFSCSVMHEALHNHYTQKSLSLSPGK
(IgG2 Fe (P107S)-linker-IL-22; linker is bolded)
SEQ ID NO: 27
VECPPCPAPPVAGPSVFLFPPKPKDTLMISRTPEVTCVVVDVSHEDPEVQFNWYVDGVEVHNAK
TKPREEQFNSTFRVVSVLTVVHQDWLNGKEYKCKVSNKGLPASIEKTISKTKGQPREPQVYTLPP
SREEMTKNQVSLTCLVKGFYPSDIAVEWESNGQPENNYKTTPPMLDSDGSFFLYSKLTVDKSRW
QQGNVFSCSVMHEALHNHYTQKSLSLSPGKASTKGPAPISSHCRLDKSNFQQPYITNRTFMLAK
EASLADNNTDVRLIGEKLFHGVSMSERCYLMKQVLNFTLEEVLFPQSDRFQPYMQEVVPFLARL
SNRLSTCHIEGDDLHIQRNVQKLKDTVKKLGESGEIKAIGELDLLFMSLRNACI
(IL-22-linker-IL-22; linker is bolded)
SEQ ID NO: 28
APISSHCRLDKSNFQQPYITNRTFMLAKEASLADNNTDVRLIGEKLFHGVSMSERCYLMKQVLNF
TLEEVLFPQSDRFQPYMQEVVPFLARLSNRLSTCHIEGDDLHIQRNVQKLKDTVKKLGESGEIKAI
GELDLLFMSLRNACIGSGGGSGGGGSGGGGSAPISSHCRLDKSNFQQPYITNRTFMLAKEASLA
DNNTDVRLIGEKLFHGVSMSERCYLMKQVLNFTLEEVLFPQSDRFQPYMQEVVPFLARLSNRLS
TCHIEGDDLHIQRNVQKLKDTVKKLGESGEIKAIGELDLLFMSLRNACI
(ERKCC)
SEQ ID NO: 29
ERKCC
(signal peptide)
SEQ ID NO: 30
MAALQKSVSSFLMGTLATSCLLLLALLVQGGAA
(human IL-22 (precursor); signal peptide is bolded)
SEQ ID NO: 31
MAALQKSVSSFLMGTLATSCLLLLALLVQGGAAAPISSHCRLDKSNFQQPYITNRTFMLAKEA
SLADNNTDVRLIGEKLFHGVSMSERCYLMKQVLNFTLEEVLFPQSDRFQPYMQEVVPFLARLSN
RLSTCHIEGDDLHIQRNVQKLKDTVKKLGESGEIKAIGELDLLFMSLRNACI
(linker)
SEQ ID NO: 32
GPGPGP
(Glu-Lys-Arg)
SEQ ID NO: 33
EKR

1. A method of preventing or treating a virus-induced organ injury or failure in an individual, comprising administering to the individual an effective amount of an IL-22 dimer.

2. The method of claim 1, wherein the virus-induced organ injury or failure is virus-induced lung injury or failure.

3. The method of claim 2, wherein the virus-induced lung injury or failure is pulmonary fibrosis, pneumonia, acute lung injury (ALI), acute respiratory distress syndrome (ARDS), Severe Acute Respiratory Syndrome coronavirus (SARS), Middle East Respiratory Syndrome coronavirus (MERS), Coronavirus disease 2019 (COVID-19), Influenza A virus subtype H1N1 (H1N1) swine flu, or Influenza A virus subtype H5N1 (H5N1) bird flu.

4-8. (canceled)

9. The method of claim 3, wherein the virus is Severe Acute Respiratory Syndrome Coronavirus 2 (SARS-CoV-2).

10-11. (canceled)

12. The method of claim 1, further comprising administering to the individual an effective amount of another therapeutic agent.

13-14. (canceled)

15. The method of claim 12, wherein the other therapeutic agent is (i) remdesivir, (ii) lopinavir and IFNα, or (iii) ritonavir and IFNα, and the virus-induced organ injury or failure is induced by SARS-CoV-2.

16. The method of claim 12, wherein the other therapeutic agent is selected from the group consisting of oseltamivir, zanamivir, peramivir, lopinavir, ritonavir, IFNα, and any combinations thereof, and the virus-induced organ injury or failure is induced by H1N1 or H5N1.

17-20. (canceled)

21. A method of promoting regeneration of injured tissue or organ due to virus infection in an individual, comprising administering to the individual an effective amount of an IL-22 dimer.

22-23. (canceled)

24. A method of treating or preventing endothelial cell injury, dysfunction, or death in an injured tissue or organ due to virus infection in an individual, comprising administering to the individual an effective amount of an IL-22 dimer.

25-26. (canceled)

27. The method of claim 1, wherein the method reduces inflammation due to virus infection in the individual.

28. The method of claim 1, wherein the IL-22 dimer comprises two monomeric subunits, and wherein each monomeric subunit comprises an IL-22 monomer and a dimerization domain.

29. The method of claim 28, wherein the IL-22 monomer is connected to the dimerization domain via a linker.

30-33. (canceled)

34. The method of claim 28, wherein the dimerization domain comprises at least a portion of an Fc fragment.

35. (canceled)

36. The method of claim 34, wherein the Fc fragment comprises the sequence of SEQ ID NO: 22 or 23.

37. The method of claim 28, wherein the IL-22 monomer comprises the sequence of SEQ ID NO: 21.

38. The method of claim 28, wherein the IL-22 monomer is N-terminal to the dimerization domain within each monomeric subunit.

39. (canceled)

40. The method of claim 28, wherein each monomeric subunit comprises the sequence of any of SEQ ID NOs: 24-27.

41. (canceled)

42. The method of claim 1, wherein the effective amount of the IL-22 dimer is about 2 μg/kg to about 200 μg/kg.

43-45. (canceled)

46. The method of claim 1, wherein the IL-22 dimer is administered intravenously, intrapulmonarily, or via inhalation or insufflation.

47-50. (canceled)
